(19) United States
(12) Patent Application Publication
(10) Pub. No.: US 2020/0131526 A1
(43) Pub. Date: Apr. 30, 2020

(54) A TRANSGENIC PLANT HAVING RESISTANCE TO A PHYTO-PATHOGENIC FUNGUS

(71) Applicant: KWS SAAT SE, Einbeck (DE)

(72) Inventors: Holger DEISING, Halle (Saale) (DE); Ely OLIVEIRA GARCIA, Manhattan, KS (US); Dietmar STAHL, Einbeck (DE); Nora TEMME, Selm (DE)

(73) Assignee: KWS SAAT SE, Einbeck (DE)

(21) Appl. No.: 15/750,736

(22) PCT Filed: Aug. 2, 2016
(86) PCT No.: PCT/EP2016/068418

§ 371 Date: Feb. 6, 2018

(30) Foreign Application Priority Data
Aug. 7, 2015 (DE) 10 2015 010 050.5

(51) Int. Cl. C12N 15/82

(52) U.S. Cl. CPC C12N 15/8282; C12N 2310/11; C12N 2310/141

(57) ABSTRACT

The present invention is directed to a transgenic plant or a part thereof comprising a DNA capable of expressing an inhibitory nucleic acid molecule capable of inhibiting the expression of (a) KRE5 and/or KRE6 gene(s) in a fungus, to such DNA, to the inhibitory nucleic acid molecule, to a vector comprising the DNA, and to methods of producing the plant, of conferring fungal resistance to the plant, or of inhibiting the expression of the KRE5 and/or KRE6 gene(s) in a fungus, to the use of the DNA for inactivating a fungus, for protecting a plant against an infection by a fungus, or for inhibiting the expression of the KRE5 and/or KRE6 gene(s) in a fungus and to a composition comprising the DNA, vector or a dsRNA capable of inhibiting the expression of (a) KRE5 and/or KRE6 gene(s) in a fungus.

Crop plants are often infected by plant-pathogenic fungi. These infections lead to profit cuts of sometimes drastic proportions. So far, the problem of fungal infection has been addressed in the interest of good agricultural practice by the use of phytosanitary measures, fungicide treatment and introduction of resistance genes by classic crossing. These measures, however, do not show a permanent protection of plants from infection caused by phytopathogenic fungi because phytosanitary measures merely result in some decreased concentrations of infectious germs, fungicide treatments frequently implicate the formation of fungicide-resistant pathogens, and, as a rule, resistance genes newly introduced by crossing are broken after only a short period of time. Some years ago, a new genetic engineering method was developed using the expression of an RNA interference (RNAi) construct to provide for resistance to infection caused by plant-pathogenic fungi forming homologous mRNAs (see, e.g., U.S. Pat. No. 7,741,531 B2, Nowara et al. 2010, Plant Cell 22, 3130-3141). This method is referred to as “host-induced gene silencing” (HIGS).

The fungal cell wall is a scaffold protecting the fungal cell from osmolysis, providing the hyphae with a form and allowing for the adhesion and deposition of enzymes and UV-protective pigments. The pores of the cell wall regulate the entrance of large molecules into the organism and provide protection against lytic enzymes of foreign organisms. To some extent, phyto-pathogenic fungi form specific infection structures, appressoria, that are stabilized by strong cell walls and that by means of a high turgor pressure allow the pathogens to enter the host tissue.

Polysaccharides account for more than 90% of the fungal cell wall, with β-1,3-glucan covalently cross-linked to chitin forming the primary scaffold, to which other β-linked polysaccharides and proteins are attached. In most fungal species, β-1,3-linked glucan is the dominating polymer, comprising between 65 and 90% of the cell wall glucan fraction. The term glucan applies to several polymers of D-glucose bound by α- and β-linkages. β-1,3-glucans are complex polysaccharides, comprising β-1,6-branches, providing for the cross-linking of the β-1,3-glucan molecules. To date, the β-1,3-glucan branching process remains unclear.

In comparison to synthesis of chitin and β-1,3-glucan, and in spite of the considerable amount of work invested, the mechanism of synthesis of β-1,6-glucan is still hypothetical. The initial milestones in understanding of β-1,6-glucan synthesis originated from mutational screens for resistance to killer strains of yeast producing the pore-forming K1 killer toxin (Bussey, 1991, Mol. Micrbiol. 5: 2339-2343; Schmitt M J and Reiter J, 2008, Biochim. Biophys. Acta-Mol. Cell Res. 1783: 1413-1417). Killer toxin activity requires binding of the toxin to β-1,6-glucan.

As defects in β-1,6-glucan synthesis interfere with toxin binding and hence confer killer toxin resistance, β-1,6-glucan synthesis genes identified through these screens are referred to as KRE genes. Interestingly, localization of the proteins encoded by these genes showed that β-1,6-glucan synthesis is initiated in the endoplasmic reticulum (ER), progresses in the Golgi apparatus, and is completed at the cell surface (Boone et al., 1990, The Journal of Cell Biology 110: 1833-1843; Shahinian S and Bussey H, 2000, Mol. Microbiol. 35: 477-489), suggesting that β-1,6-glucan synthesis is secretory pathway-based. Today, several genes involved in β-1,6-glucan synthesis have been identified in yeast, including the two genes KRE5 and KRE6 (Shahinian and Bussey, 2000).

KRE5 encodes a luminal ER protein containing a C-terminal ER retention signal. Based on its limited but yet significant similarity to UDP-glucose:glycoprotein glucosyltransferases Kre5p has been proposed to function as a glucosyltransferase involved in initiation of β-1,6-glucan synthesis. However, UDP-glucose:glycoprotein glucosyltransferase activity catalyzing the attachment of a glucose residue to the N-glycosyl chains of target proteins in the ER has not been found in yeast (Fernandez et al, 1994, J. Biol. Chem. 269: 30701-30706). Alternatively, Kre5p has been proposed to be a glycosyltransferase which may β-1,6-glucosylate the glycosylphosphatidylinositol (GPI) moiety of cell wall proteins in the ER, which is eventually required for cross-linking of the cell wall network. Several lines of evidence support this hypothesis but unambiguous biochemical proof is still missing (Shahinian and Bussey, 2000). Although genetic and chemical data highlight the role of Kre5 in β-1,6-glucan synthesis, the biochemical activity and function of Kre5 remain hypothetical. KRE6 and its functional homolog SKN1 encode putative membrane-integral glucohydrolases localized in the Golgi apparatus. KRE6-deficient yeast mutants show a reduction of cell wall β-1,6-glucan contents by 50%. Kre6 and Skn1 show significant sequence similarity to several bacterial β-1,3-glucanases (Shahinian and Bussey, 2000, and references therein), suggesting that Kre6 may have a processing rather than an elaborating function.

Based on the localization and predicted function of Kre5 and Kre6 proteins, Shahinian and Bussey, 2000, introduced a model of transient glucosylation with addition of β-1,6-linked glucan residues in the ER by Kre5 and their removal in the Golgi by Kre6, with both steps being required for correct cell wall targeting, completion of β-1,6-glucan synthesis and cross-linking with other cell wall polymers on the cell surface.

β-1,3-glucans are attached to the plasma membrane by a glycoprotein complex called glycosylphosphatidylinositol (GPI)-proteins (Heilmann et al., 2012, PLoS Pathogens 8: e1003050). The most of the GPI-proteins synthesis process occurs in the ER and several proteins are involved in this process.

While significant effort has been taken to mechanistically unravel β-1,6-glucan synthesis, information on the role of this polymer and the genes required for its synthesis is limited to yeasts and dimorphic fungi with medical relevance. Notably, seven KRE family genes of the human pathogen Cryptococcus neoformans (Gilbert et al., 2010, Mol. Microbiol. 76: 517-534) are functionally characterized. As expected from the work with S. cerevisiae, Δkre5 and Δkre6Δskn1 mutants of this fungus had significantly reduced contents of β-1,6-glucan, and were sensitive to elevated temperature and chemicals interfering with cell wall synthesis such as Calcofluor White and Congo Red.

Thus, even though the function of β-1,6-glucan as well as the requirement of KRE5 and KRE6 for synthesis of this cell wall polymer and for virulence is documented in the above mentioned pathogens infecting mammals, the role of these genes in the infection process of filamentous plant pathogenic fungi is unknown. In contrast to fungi infecting animals, plant pathogens differentiate several morphologically distinct infection cells, depending on the lifestyle of the pathogen. Particularly, these infection structures show marked differences in composition and surface exposure of cell wall polymers (de Jonge et al., 2010, Science 329: 953-955; Oliveira-Garcia and Deising, 2013, Plant Cell 25: 2356-2378; Mentlak T A et al., 2012, Plant Cell 24: 322-335). Furthermore, it is unknown whether or not formation of β-1,6-links in cell wall glucan polymers is essential for cell wall function in infection structures. Furthermore, it is unknown whether or not infection structure-specific regulation of linear β-1,3- or branched β-1,3-β-1,6-glucans is required for the establishment of a compatible parasitic interaction with the host plant (Oliveira-Garcia and Deising, 2013).

Various working groups have examined biosynthesis genes of the fungal cell wall as to their suitability as potential target genes for an HIGS approach for pathogen resistance. The inhibition of chitin synthesis of Fusarium graminearum by HIGS in transgenic wheat plants resulted in an increased resistance specifically against F. graminearum (Cheng et al., 2015) (Plant Biotechnology Journal, doi: 10.1111/pbi.12352). In their scientific paper, Nowara et al. 2010 addressed pathogenic target genes that are relevant for cell wall biogenesis with HIGS constructs in plants. In that case, these were the two β-1,3-glucanosyl transferases GTF1 and GTF2 of the corn mildew virus Blumeria graminis. These genes, however, are not well suited for HIGS use, since almost all examined fungi with annotated genome have at least two β-1,3-glucanosyl transferases and these enzymes are involved only in late stages of the cell wall biosynthesis. These two reasons suggest that β-1,3-glucanosyl transferase genes are not HIGS candidate genes that could be preferably and successfully used in molecular plant cultivation.

There exists, thus, the need for an appropriate fungal target gene, which is suitable to confer broad and permanent fungal resistance to plants.

In a first aspect, the present invention relates to a transgenic plant or a part thereof comprising as transgene a DNA capable of expressing an inhibitory nucleic acid molecule capable of inhibiting the expression of (a) KRE5 and/or KRE6 gene(s) in a fungus.

In an embodiment thereof, the transgenic plant or the part thereof comprises as transgene an expression cassette comprising the DNA.

In an embodiment thereof, the DNA or the expression cassette is stably integrated into the genome of the plant or present in the plant or the part thereof on a vector.

In an embodiment thereof, the inhibitory nucleic acid molecule is an antisense RNA or dsRNA, whereby the dsRNA is preferably hpRNA, siRNA or miRNA.

In an embodiment thereof, the DNA encodes an RNA molecule in sense direction and an RNA molecule in antisense direction, wherein the RNA molecule in sense direction or the RNA molecule in antisense direction are substantially complementary or reverse complementary to the KRE5 and/or KRE6 gene(s) or part(s) thereof, in particular the RNA molecule in antisense direction is substantially reverse complementary to the RNA molecule in sense direction, and the RNA molecule in sense direction and the RNA molecule in antisense direction are able to build a dsRNA.

In an embodiment thereof, the length of the antisense RNA, the dsRNA, the RNA molecule in sense direction or the RNA molecule in antisense direction is at least 15, 16, 17, 18, 19 or 20 contiguous nucleotides, preferably at least 21, 22, 23, 24, 25, 30, 40, 50, 60, 70, 80, 90 or 100 contiguous nucleotides, and more preferably at least 150, 200, 250, 300, 350, 450, 500, 600, 700, 800, 900 or 1000 contiguous nucleotides. The upper limit of the molecule is determined by the length of the KRE5 and KRE6 genes or mRNAs. Most preferably, the length is 300-2000, such as 200-1000, 300-700, 400-500, 440-470, or 450-460 contiguous nucleotides. Exemplarily, the length may be around 455 contiguous nucleotides such as 456 contiguous nucleotides.

Consequently, by “part” of the KRE5 and/or KRE6 gene(s) is meant any part of the genes to which an aRNA or an RNA molecule in antisense direction hybridizes and inhibits expression thereof. According to the above, the “part” of the genes may be at least 15, 16, 17, 18, 19 or 20 contiguous nucleotides, preferably at least 21, 22, 23, 24, 25, 30, 40, 50, 60, 70, 80, 90 or 100 contiguous nucleotides, and more preferably at least 150, 200, 250, 300, 350, 450, 500, 600, 700, 800, 900 or 1000 contiguous nucleotides in length, but is below the length of the KRE5 and KRE6 genes or mRNAs. Most preferably, the length is 300-2000, such as 200-1000, 300-700 or 400-500 contiguous nucleotides. Exemplarily, the length may be around 450 contiguous nucleotides such as 456 contiguous nucleotides. The “part” of the genes may be in any region of the KRE5 gene or KRE6 gene or of the KRE5 mRNA or KRE6 mRNA, so that upon hybridizing of the aRNA or RNA molecule in antisense direction, the transcription of the gene or the translation of the mRNA is inhibited. The “part” may be in a regulatory region such as the promoter region or terminator regulatory sequence, in exon sequences, in intron sequences or along the whole mRNA, as long as the transcription of the gene or the translation of the mRNA is inhibited.

In an embodiment thereof, the expression of the inhibitory nucleic acid molecule is controlled by a promoter, preferably an inducible promoter, more preferably a pathogen inducible and/or tissue specific promoter. The inducible promotor can be a chimeric promoter which is composed of a plurality of elements and does not occur as such nature. It may contain a minimal promoter and include, upstream from the minimal promoter, at least one cis-regulatory element which serves as a binding site for specific trans-acting factors (e.g. transcription factors). A chimeric promoter can be designed according to the desired requirements and is induced or repressed by different factors. The selection of the cis-regulatory element or a combination of cis-regulatory elements is critical for the specificity or the activity level of a promoter. A cis-regulatory element in a chimeric promoter is either heterologous to the minimal promoter used, i.e. the cis-regulatory element is derived from a different organism or from a different species to that of the minimal promoter used, or a cis-regulatory element in a chimeric promoter is homologous to the minimal promoter used, i.e. the cis-regulatory element and the minimal promoter also occur combined in a natural promoter, however the cis-regulatory element is localized itself or as an additional element within the chimeric promoter in a genetic environment that is different from the natural promoter. A chimeric promoter thus also means a (natural) promoter that was altered by multimerization of at least one cis-regulatory element. Pathogen inducible chimeric promoters are known from the prior art (WO 00/29592; WO 2007/147395; WO 2013/091612).

Each of the embodiments of the first aspect refers back to each preceding embodiment.

In a second aspect, the present invention relates to a DNA capable of expressing an inhibitory nucleic acid molecule capable of inhibiting the expression of (a) KRE5 and/or KRE6 gene(s) in a fungus, whereby preferably the DNA is as defined above in the first aspect of the invention, or an expression cassette comprising the DNA.

In a third aspect, the present invention relates to an inhibitory nucleic acid molecule capable of inhibiting the expression of (a) KRE5 and/or KRE6 gene(s) in a fungus, whereby preferably the inhibitory nucleic acid molecule is as defined above in the first aspect of the invention.

In a fourth aspect, the present invention relates to a vector comprising the DNA or the expression cassette as referred to in the second aspect of the invention.

In a fifth aspect, the present invention relates to a method of producing the transgenic plant or the part thereof as referred to in the first aspect of the invention, comprising the following steps: introducing into at least a cell of the plant the DNA or the expression cassette as referred to in the second aspect of the invention or the vector as referred to in the fourth aspect of the invention, and regenerating the transgenic plant from the at least one cell.

In a sixth aspect, the present invention relates to a method of conferring fungal resistance to a plant or the part thereof comprising the following steps: introducing into the plant or the part thereof the DNA or the expression cassette as referred to in the second aspect of the invention or the vector as referred to in the fourth aspect of the invention, and causing expression of the DNA or the expression cassette.

In a seventh aspect, the present invention relates to a method of inhibiting the expression of the KRE5 and/or KRE6 gene(s) in a fungus, comprising: applying the DNA or the expression cassette as referred to in the second aspect of the invention or the vector as referred to in the fourth aspect of the invention to the fungus or to a plant or a part thereof.

In an eight aspect, the present invention relates to a use of the DNA or the expression cassette as referred to in the second aspect of the invention or the vector as referred to in the fourth aspect of the invention for inactivating a fungus, while contacting a plant or a part thereof; for protecting a plant against an infection by a fungus; or for inhibiting the expression of the KRE5 and/or KRE6 gene(s) in a fungus.

In a ninth aspect, the present invention relates to a composition comprising the DNA or the expression cassette as referred to in the second aspect of the invention or the vector as referred to in the fourth aspect of the invention or a dsRNA capable of inhibiting the expression of (a) KRE5 and/or KRE6 gene(s) in a fungus.

The invention also relates to a transgenic plant or a part thereof comprising a DNA or an expression cassette comprising the DNA, wherein the DNA encodes an aRNA or dsRNA directed against the KRE5 DNA and/or mRNA, wherein preferably the dsRNA is hpRNA, siRNA or miRNA, or the aRNA is part of the dsRNA, siRNA or miRNA.

The invention also relates to a transgenic plant or a part thereof comprising a DNA or an expression cassette comprising the DNA, wherein the DNA encodes an aRNA or dsRNA directed against the KRE6 DNA and/or mRNA wherein preferably the dsRNA is hpRNA, siRNA or miRNA, or the aRNA is part of the dsRNA, siRNA or miRNA.

The invention also relates to a transgenic plant or a part thereof comprising a DNA or an expression cassette comprising the DNA, wherein the DNA encodes an aRNA or dsRNA directed against the KRE5 and an aRNA or dsRNA directed against the fungal KRE6 DNA and/or mRNA, wherein preferably the dsRNA is hpRNA, siRNA or miRNA, or the aRNA is part of the dsRNA, siRNA or miRNA.

The invention also relates to a transgenic plant or a part thereof comprising a DNA or an expression cassette comprising the DNA, wherein the DNA comprises an antisense and a sense sequence whereby the antisense sequence is capable of hybridizing to fungal KRE5 mRNA.

The invention also relates to a transgenic plant or a part thereof comprising a DNA or an expression cassette comprising the DNA, wherein the DNA comprises an antisense and a sense sequence whereby the antisense sequence is capable of hybridizing to fungal KRE6 mRNA.

The invention also relates to a transgenic plant or a part thereof comprising a DNA or an expression cassette comprising the DNA, wherein the DNA comprises an antisense sequence against the KRE5 mRNA and an antisense sequence against the KRE6 mRNA and preferably sense sequences thereto, wherein the antisense sequences are capable of hybridizing to fungal KRE5 mRNA and KRE6 mRNA, respectively.

The invention also relates to a transgenic plant or a part thereof comprising a DNA or an expression cassette comprising the DNA, wherein the DNA encodes an RNA molecule in sense direction and an RNA molecule in antisense direction, wherein the RNA molecule in sense direction or the RNA molecule in antisense direction are present on one RNA molecule.

The invention also relates to a transgenic plant or a part thereof comprising a DNA or an expression cassette comprising the DNA, wherein the DNA encodes an RNA molecule in sense direction and an RNA molecule in antisense direction, wherein the RNA molecule in sense direction or the RNA molecule in antisense direction are present on different RNA molecules.

The invention also relates to a transgenic plant or a part thereof comprising a DNA or an expression cassette comprising the DNA, wherein the DNA encodes an RNA molecule in sense direction and an RNA molecule in antisense direction, wherein the RNA molecule is siRNA or miRNA.

While the requirement for KRE5 and KRE6 for synthesis of β-1,6-glucan and for virulence is well-known in the art with respect to mammals, there was to date no data whether KRE5 and KRE6 would also play a role with respect to pathogens infecting plants. Moreover, there was to date no data as to the suitability of KRE5 and KRE6 as target genes in gene silencing approaches, let alone in an HIGS approach, for broad and permanent pathogen resistance in plants.

The present inventors have surprisingly demonstrated that inhibition of fungal KRE5 and/or KRE6 gene(s) by a polynucleotide introduced into a plant confers resistance in the plant to fungi via gene silencing. The polynucleotide causes cessation of infection, growth, development, reproduction and/or pathogenicity and eventually results in the death of the fungal organism.

The invention demonstrates for the first time that the disturbance of the β-1,6-glucan synthesis in a phyto-pathogenic fungus providing for the cross-linking of the β-1,3-glucan chains leads to a massive inhibition of fungal growth, a reduced formation of conidia and to apathogenicity. It has been shown that the KRE5-encoded UDP glucose glycosyl transferase catalyzes the first biosynthesis step of the β-1,6-glucan synthase and that KRE6 encodes a protein involved in the synthesis of both β-1,3-glucan and β-1,6-glucan. As an example, the KRE5 and KRE6 genes of Colletotrichum graminicola were isolated. The identity of the genes was functionally confirmed by the complementation of the yeast mutants Δkre5 and Δkre6 for C. graminicola. Analoguously, the same procedure may be applied to the KRE5 and KRE6 genes of other pathogens. Surprisingly, the separate inhibition of the KRE5 expression and the KRE6 expression in C. graminicola strains resulted not only in the reduction of β-1,6-glucan synthesis but also in a decrease of β-1,3-glucan synthesis via a decreased transcription of GLS1. Based on the co-regulation of the β-1,3-glucan and β-1,6-glucan syntheses that was observed for the first time, the use of the KRE5 and/or KRE6 gene(s) as HIGS target genes is more effective than the use of GLS1 alone.

Moreover, by using reverse genetic methods, the inventors were able to show that the KRE5 and KRE6 genes are excellent HIGS target genes capable of selectively interrupting the beta-1,6-glucan biosynthesis and suitable for generating plants permanently resistant against pathogens. The excellent suitability of these target genes is substantiated by several properties. Firstly, these are single-copy genes such that in case of an RNAi approach it can be excluded that a second allele will save the host-induced gene silencing. Secondly, these are genes that are engaged in very early stages of the cell wall biosynthesis so that no polymer precursors develop that would be able to partially save the HIGS phenotype. Thirdly, and this is a crucial point, no complete reduction of transcript concentrations is required in order to induce apathogenicity.

The identified genes may serve to generate plants that address individual target genes or also plants that use combinations of the presently presented genes of plant-pathogenic fungi as targets. Since the reverse-genetically verified genes identified herein have homologues in the genomes of all economically relevant fungi, these targets may be used to confer permanent resistance to various crop species against a wide variety of plant-pathogenic fungi.

KRE5 and KRE6 nucleic acid and amino acid sequences are known in the art for a series of fungi. The protein and nucleotide sequences of a series of fungal KRE5 and KRE6 are listed in the accompanying sequence listing under SEQ ID NOs: 1 to 232, 375 and 376.

KRE5 and KRE6 proteins of filamentous fungi show a sequence identity of about at least 60% and 69%, respectively, however, protein sequences largely differ between various clades and may be as low as 23% and 30%, respectively, between S. cerevisiae and C. graminicola. According to the present invention, KRE5 and KRE6 genes which are comprised by the present invention as target genes in gene silencing approaches are those which are known in the art as fungal KRE5 and KRE6 genes, respectively. In the exemplary part a series of KRE5 genes and KRE6 genes and proteins in various fungi are listed and characterized by their accession numbers as available from the NCBI database (National Centre for Biotechnology Information; National Library of Medicine 38A, Bethesda, Md. 20894, USA; www.ncbi.nih.gov). Moreover, fungal KRE5 and KRE6 genes which are comprised by the present invention as target genes are characterized by their degree of identity to already known KRE5 and KRE6 genes. Thereby, fungal KRE5 and KRE6 genes are comprised as target genes by the present invention if they show a degree of identity to already known, or to those identified in the future, KRE5 and KRE6 genes of fungal organisms of at least 60, 70, 80, 90, 95 or 99%. Alternatively, fungal KRE5 and KRE6 genes are comprised by the present invention as target genes if they hybridize under stringent conditions to a fungal KRE5 and KRE6 gene, respectively, as known in the art. Additionally, a KRE5 protein encoded by a KRE5 target gene as comprised by the present invention has UDP glucose glycosyltransferase activity and catalyses the first biosynthesis step of the β-1,6-glucan synthesis. All KRE5 proteins are characterized by a clear secretion signal at the N-terminal region and two different glycosyltransferase domains at the conserved C-terminal region. All KRE5 proteins contain a C-terminal tetrapeptide for retention in the endoplasmic reticulum. Moreover, all Colletotrichum species and Neurospora crassa show an IDEL C-terminal tetrapeptide, the majority of filamentous ascomycetes shows a KDEL and dimorphic fungi show preferentially a HDEL C-terminal tetrapeptide. A KRE6 protein encoded by a KRE6 target gene as comprised by the present invention is a membrane-integral glycohydrolase protein. The vast majority of KRE6 proteins contain a single transmembrane domain at the N-terminal region and a prominent central glycohydrolase 16/KRE6 core domain.

Alternativley, identification of a KRE5 or KRE6 protein may occur via a consensus sequence. Alignment of protein sequences of various fungi of the ascomycota and basidiomycota revealed conserved sequences, in particular in the UDP-glucose:glycoprotein glucosyltransferase domain and the glucosyl-transferase family 8-like domain of the KRE5 protein and in the glycosyl hydrolases family 16 domain of the KRE6 protein. Based on homologous regions, consensus sequences can be designed which may be used to identify KRE5 or KRE6 proteins in other fungi, preferably in other fungi of the ascomycota and basidiomycota categories. Accordingly, KRE5 and KRE6 genes which are comprised by the present invention as target genes in gene silencing approaches are those which comprise consensus sequences identified as indicated above, preferably which comprise consensus sequences derived from the UDP-glucose:glycoprotein glucosyltransferase domain and/or the glucosyltransferase family 8-like domain of the KRE5 protein or the glycosyl hydrolases family 16 domain of the KRE6 protein.

As used herein, a fungus is any fungus comprising (a) KRE5 and/or KRE6 gene(s). Preferably, the fungus is selected from the group consisting of ascomycota, basidiomycota, zygomycota, chytridiomycota, blastocladiomycota, neocallimastigomycota, and glomeromycota. More preferably the fungus is a phytopathogenic fungus. Still more more preferably the fungus is selected from a genus comprising the KRE5 gene such as the genus Alternaria; Ashbya; Beauveria; Bipolaris; Botryotinia; Colletotrichum; Cladosporium; Claviceps; Cordyceps; Cryptococcus; Diplocarpon; Fusarium; Gaeumannomyces; Geotrichum; Leptosphaeria; Magnaporthe; Metarhizium; Neurospora; Pyrenophora; Puccinia; Trichoderma; Sclerotinia; Ustilago; Verticillium; Zygosaccharomyces; Blumeria; Chaetomium; Chaetomium; Endocarpon; Exophiala; Lachancea; Marssonina; Myceliophthora; Mycosphaerella; Macrophomina; Penicillium; Podospora; Paracoccidioides; Pseudopeziza; Pyrenophora; Phaeosphaeria; Sclerotinia; Sordaria; Schizsacchaormyces, Zygosaccharomyces; Talaromyces; Thielavia; Trichoderma; Venturia, and Pseudocercospora; and still more preferably the fungus is selected from Alternaria solani; Aspergillus flavus; Alternaria alternata; Aphanomyces cochlioides; Ashbya gossypii; Beauveria bassiana; Bipolaris maydis; Bipolaris sorokiniana; Botryotinia fuckeliana; Botrytis cinerea; Cercospora beticola, Cercospora kikuchii; Cercospora sojina; Cercospora zea maydis; Colletotrichum graminicola; Colletotrichum gloeosporioides; Colletotrichum orbiculare; Claviceps purpurea; Cordyceps militaris; Diplodia maydis; Drechslera glycines; Drechsiera oryzae; Erysiphe betae; Exserohilum turcicum; Fusarium fujikuroi; Fusarium graminearum; Fusarium moniliforme; Fusarium oxysporum; Fusarium solani; Gaeumannomyces graminis; Leptosphaeria maculans; Magnaporthe oryzae; Microsphaera diffusa; Penicillium; Pyrenophora teres; Pyrenophora tritici-repentis; Puccinia graminis; Trichoderma virens; Sclerotinia sclerotiorum; Ustilago maydis; Ustilago hordei; Verticillium alfalfa; Verticillium dahlia; or the fungus is selected from a genus comprising the KRE6 gene such as Blumeria graminis; Chaetomium globosum; Colletotrichum higginsianum; Colletotrichum orbiculare; Coccidioides posadasi; Endocarpon pusillum; Exophiala dermatitidis; Lachancea thermotoleran; Marssonina brunnea; Myceliophthora thermophile; Metarhizium acridum; Macrophomina phaseolina; Monographella albescens; Paracoccidioides brasiliensis; Phaeoshaeria maydis; Phakopsora pachyrhizi; Pyrenophora tritici-repentis; Phaeosphaeria nodorum; Puccinia graminis; Puccinia polysora; Puccinia striiformis; Puccinia triticina; Pyricularia oryzae; Ramularia beticola, Rhizoctonia oryzae; Rhizoctonia solani; Sclerotium rolfsii; Saccharomyces arboricola; Sordaria macrospora; Schizsacchaormyces japonicus, Zygosaccharomyces rouxii; Talaromyces marneffei; Talaromyces stipitatus; Thielavia terrestris; Venturia inaequalis; Verticillium albo-atrum; Verticillium dahlia; Zymoseptoria tritici; and Pseudocercospora fijiensis. The fungus may also be selected from the genus Candida such as Candida albicans; Candida glabrata; Candida orthopsilosis; Candida parapsilosis; or Candida tropicalis; Saccharomyces; and Coccidioides. KRE5 and/or KRE6 gene(s) of non-phytopathogenic fungi, as e.g. mentioned herein, may serve in the finding of not yet identified KRE5 and/or KRE6 gene(s) of phytopathogenic fungi, e.g. via consensus sequences.

Also included within the term “fungus” are fungus-like eukaryotic microorganisms such as the oomycota or oomycetes which include notorious pathogens of plants, causing devastating diseases such as late blight of potato and sudden oak death. Examples of oomycetes which are included by the present invention are Phytophthora infestans, Phytophthora palmivora, Phytophthora sojae. Oomycetes are included within the present invention and are, for the purposes of the present invention, included within the term “fungal” or “fungus”.

As used herein, the term “phyto-pathogenic” or “pathogenic” refers to a fungus which causes a disease in a plant.

The methods as comprised by the present invention which are used to inhibit the expression of KRE5 and/or KRE6 gene(s) in a fungus are known to the skilled person under the term “gene silencing”. “Gene silencing” is a general term used to describe the regulation of gene expression. In particular, this term refers to the ability of a cell to prevent the expression of a gene in the cell. Gene silencing can occur during either transcription or translation of a gene. The application of gene silencing methods in plants is well known in the art. Reference is made to Plant Gene Silencing—Methods and Protocols, Mysore K. S and Senthil-Kumar M. (eds.), Springer Protocols, Humana Press, 2015, which provides the reader with a comprehensive review of various gene silencing methodologies and its applications. The skilled person may apply any methods described in this document or elsewhere which are suitable to achieve gene silencing in a plant and in a fungus.

Generally used gene silencing methodologies in plants include host-induced gene silencing (HIGS) which is a method where a plant controls pathogens by RNA interference (RNAi) and exhibits an enhanced resistance when carrying suitable gene silencing constructs. RNA interference is a natural process used by cells in many eukaryotes to regulate gene expression. RNA interference is a vital part of the immune response to viruses and other foreign genetic material, especially in plants. For inducing RNAi in a cell or organism, dsRNA with a sequence substantially complementary to a gene of interest is synthesized either within a cell or organism or it is synthesized outside the cell or organism and introduced into the cell or organism, where it is recognized as exogenous genetic material and activates the RNAi pathway. The double-stranded molecule is cut into small double-stranded fragments by an enzyme called Dicer. These small fragments, which include small interfering RNAs (siRNA) and microRNA (miRNA), are often 19-40 nucleotides in length. The fragments integrate into a multi-subunit protein called the RNAi induced silencing complex (RISC). One strand of the molecule, the guide strand or antisense strand, binds to RISC, while the other strand, the passenger strand or sense strand, is degraded. The guide strand pairs with a complementary sequence in an mRNA molecule and induces cleavage by Argonaute, the catalytic component of the RISC complex, thereby preventing it from being used as a translation template.

Other gene silencing methodologies in plants use antisense oligonucleotides which are short nucleic acid fragments that bind to substantially complementary target mRNA molecules. These molecules are single-stranded RNA molecules generally 15-25 nucleotides long. Gene silencing in plants can also occur via ribozymes which are catalytic RNA molecules used to inhibit gene expression. These molecules work by catalyzing specific biochemical reactions such as cleaving mRNA molecules. Several types of ribozyme motifs exist, including hammerhead, hairpin, hepatitis delta virus, group I, group II, leadzyme, Varkud satellite (VS) and RNase P ribozymes. The general catalytic mechanism used by ribozymes is similar to the mechanism used by protein ribonucleases. These catalytic RNA molecules bind to a specific site and attack the neighboring phosphate in the RNA backbone with their 2′ oxygen, which acts as a nucleophile, resulting in the formation of cleaved products with a 2′3′-cyclic phosphate and a 5′ hydroxyl terminal end.

The preferred method used in the present invention is the HIGS method. Moreover, any other gene silencing method known in the art may be used for inhibiting the expression of a fungus while being in contact with a plant.

For the production of fungus resistant plants in the present invention, a DNA capable of expressing an inhibitory nucleic acid molecule capable of inhibiting the expression of (a) KRE5 and/or KRE6 gene(s) in a fungus is selected and introduced into the plant or a part thereof.

As used herein, “a DNA capable of expressing an inhibitory nucleic acid molecule” is any DNA molecule which results in an inhibitory nucleic acid molecule. Preferably, the DNA comprises an antisense sequence which is substantially complementary to contiguous stretches of the fungal KRE5 and/or KRE6 mRNA(s) or part(s) thereof. Upon transcription, an inhibitory molecule, preferably an RNA molecule, is produced which comprises the antisense sequence which is capable of hybridizing to the fungal KRE5 and/or KRE6 mRNA(s) or part(s) thereof. The RNA molecule may be an mRNA molecule, a single-stranded antisense RNA, or a dsRNA, for example a hpRNA, siRNA or miRNA. The DNA may comprise one or more than one antisense sequence(s). Thus, the DNA may comprise one or more than one antisense sequence(s) against fungal KRE5 gene(s) and/or RNA(s), or one or more than one antisense sequence(s) against fungal KRE6 gene(s) and/or RNA(s), or one or more than one antisense sequence(s) against fungal KRE5 and one or more than one antisense sequence(s) against fungal KRE6 gene(s) and/or RNA(s). Additionally and preferably, the DNA may comprise one or more than one sense sequence(s) which is (are) substantially complementary to the antisense sequence(s). The antisense and sense sequences may be present on the same or on different DNA strands. Upon transcription, inhibitory molecule(s) comprising the antisense and possibly sense sequences are generated. Alternatively, the DNA may encode a catalytic RNA or ribozyme or may encode an inhibitory nucleic acid molecule which is or encodes a repressor molecule. Such a repressor molecule may, e.g., act by inhibiting the access of proteins, which are necessary for transcription or translation, to the fungal KRE5 and/or KRE6 gene(s) and/or RNA(s). The DNA may be double-stranded or single-stranded and is preferably double-stranded.

The DNA capable of expressing an inhibitory nucleic acid molecule may be present within an expression cassette. As used herein, an “expression cassette” is a nucleic acid molecule which is composed of one or more genes or genetic sequences and the sequences controlling their expression. An expression cassette may contain a promoter regulatory sequence, also designated promoter, operably linked to an open reading frame or another genetic sequence, and a 3′ untranslated region that may contain a polyadenylation site. The promoter directs the machinery of the cell to make RNA and/or protein. As used herein, “operably linked” means that expression of the linked DNA sequences occurs in the plant. An expression cassette may be part of a vector used for cloning and introducing the DNA into a cell.

As used herein, an “antisense sequence” is a sequence which is substantially complementary to any contiguous stretch of (a) fungal KRE5 and/or KRE6 mRNA(s) or part(s) thereof. The antisense sequence is selected such that it hybridizes to a contiguous sequence element of the KRE5 and/or KRE6 mRNA(s) and inhibits the translation thereof. The antisense sequence has a length which allows inhibition of translation of the KRE5 and/or KRE6 mRNA(s) in the fungus. The antisense sequence as comprised by an aRNA which inhibits the KRE5 and/or KRE6 mRNA(s) via the antisense mechanism has a length of at least 15 nucleotides and may extend to hundreds of nucleotides or over the whole length of the KRE5 or KRE6 mRNA(s). The preferred length of the antisense sequence as comprised by an aRNA is 15 to 300, 15 to 200, 15 to 100 or 15 to 50 nucleotides and most preferably 15 to 25 nucleotides. The antisense sequence as comprised by dsRNA which induces the RNAi machinery has a length of at least 19 nucleotides and may extend to hundreds of nucleotides or over the whole length of the KRE5 or KRE6 mRNA(s). The preferred length of the antisense sequence as comprised by dsRNA is 19 to 300, 19 to 200, 19 to 100 or 19 to 50 nucleotides and most prefereably 19 to 25 nucleotides. The antisense sequence may be directed against the coding part or the regulatory part of the fungal KRE5 and/or KRE6 mRNA(s).

The selection of a suitable antisense sequence can be performed by methods known in the art. For RNAi constructs, RNAi programs like Emboss siRNA prediction (http://emboss.sourceforge.net/apps/release/6.6/emboss/apps/sirna.html) are available which identify suitable sequence elements of the KRE5 and/or KRE6 genes.

As used herein, a “sense sequence” is a sequence which is substantially complementary to an antisense sequence. Due to the substantial complementarity of the sequences, the antisense and sense sequences hybridize with each other.

The term “complementary” includes “complementary” as well as “reverse complementary”. “Reverse complementary” means that the nucleotides of a reverse complementary sequence are, as regards their 5′ to 3′ extension, complementary and ordered in a mirrored fashion with respect to a polynucleotide sequence. Thus, independent of whether antisense and sense sequences are on the same strand or on different strands (DNA or RNA), they need to be reverse complementary allowing them to form a double-strand either by intramolecular hybridization or inter-molecular hybridization, respectively.

The term “capable of expressing” means that the DNA is the starting substance which is transferred within the plant cell into a nucleic acid molecule which is capable of inhibiting the expression of (a) KRE5 and/or KRE6 gene(s) in a fungus. Preferably, this term comprises the transcription of the DNA into an RNA molecule, which can hybridize to (a) KRE5 and/or KRE6 gene(s) or (a) KRE5 and/or KRE6 mRNA(s) in a fungus and inhibit the expression thereof.

As used herein, “an inhibitory nucleic acid molecule” or “an inhibitory nucleic acid molecule capable of inhibiting the expression of (a) KRE5 and/or KRE6 gene(s)” is any nucleic acid molecule which inhibits the transcription and/or translation of (a) KRE5 and/or KRE6 gene(s) or (a) KRE5 and/or KRE6 RNA(s) in a fungus. Inhibition may occur in the coding part of the KRE5 and/or KRE6 gene(s) or KRE5 and/or KRE6 RNA(s) in the fungus or the regulatory part located in the regions 5′ and/or 3′ to the gene(s) or RNA(s). Preferably, the inhibition is by hybridizing to (the) KRE5 and/or KRE6 DNA(s) or mRNA(s) in the fungus due to substantial complementarity, so that the KRE5 and/or KRE6 DNA(s) and/or RNA(s) cannot be transcribed and/or translated, respectively. Inhibition may also be achieved by degrading the hybridized KRE5 and/or KRE5 gene(s) or RNA(s). Preferably, the inhibitory nucleic acid molecule is an RNA molecule which is transcribed from the DNA capable of expressing an inhibitory nucleic acid molecule. More preferably, the inhibitory nucleic acid molecule is a single-stranded antisense RNA (aRNA) which comprises an antisense sequence which is substantially complementary to the fungal KRE5 and/or KRE6 mRNA(s) and which hybridizes thereto and inhibits the translation thereof. Alternativley, the inhibitory nucleic acid is an RNA molecule in sense direction and an RNA molecule in antisense direction. The RNA molecule in sense direction and the RNA molecule in antisense direction may be one RNA molecule or may be different RNA molecules. Due to their inverse substantial complementarity, the sense and antisense sequences hybridize with each other and form dsRNA. The dsRNA such as siRNA or miRNA may be composed of two separate strands or may be one strand such as hpRNA. In a further alternative, the inhibitory nucleic acid molecule is a dsRNA, namely siRNA or miRNA, which forms within the plant during the RNAi procedure, whereby dsRNA transcribed from the DNA induces the RNAi mechanism and is processed to the siRNA or miRNA. Alternatively, the inhibitory molecule may be a catalytic RNA or ribozyme. In a further alternative, the inhibitory nucleic acid molecule may be or encode a repressor molecule which functions by inhibiting the transcription and/or translation of the KRE5 and/or KRE6 gene(s) and/or RNA(s) in the fungus. For example, the repressor molecule functions by inhibiting the access of proteins, which are necessary for transcription or translation, to the fungal KRE5 and/or KRE6 gene(s) and/or RNA(s).

As used herein, the term “capable of inhibiting the expression of (a) KRE5 and/or KRE6 gene(s)” or “inhibiting the expression of (a) KRE5 and/or KRE6 gene(s)” means that the inhibitory nucleic acid molecule prevents that the fungal KRE5 and/or KRE6 gene(s) or mRNA(s) are processed further, such as transcribed or translated. Prevention may occur by any kind of inhibition such as hybridization to the KRE5 and/or KRE6 gene(s) and/or RNA(s), cleaving of the KRE5 and/or KRE6 gene(s) and/or RNA(s), repressing the transcription or translation of the KRE5 and/or KRE6 gene(s) and/or RNA(s) or catalytic actions as performed by ribozymes. Preferably, the nucleic acid molecule can hybridize or hybridizes to (a) KRE5 and/or KRE6 gene(s) and/or (a) KRE5 and/or KRE6 mRNA(s) and prevents the further processing thereof such as transcription of DNA or translation of mRNA. Prevention occurs either by blocking the KRE5 and/or KRE6 gene(s) or mRNA(s) so that downstream actions cannot be performed or by degrading the KRE5 and/or KRE6 gene(s) or mRNA(s) by host-specific enzymes. A reduction of expression of at least 40%, 50%, 60%, 70%, 80%, 90%, 95% or of 100% for the KRE5 gene and/or of at least 40%, 50%, 60%, 70%, 80%, 90%, 95% or of 100% for the KRE6 gene, as compared to expression in a non-transgenic plant of identical phenotype, allows scoring the transgenic plant as being resistant or as having resistance to a fungus. Preferably, the damages on the plant are reduced by at least 50%, 60%, 70%, 80%, 90% or by 100% in a resistant transgenic plant according to the present invention as compared to a non-transgenic plant of identical phenotype. Inhibition of expression of (a) KRE5 and/or KRE6 gene(s) results in the reduction of β-1,6-glucan synthesis and a decrease of β-1,3-glucan synthesis via a decreased transcription of GLS1.

Each fungus has its characteristic picture of damages which it causes with a host plant. The damages are e.g. visible in the form of leaf spots or root rot (dry texture, concentric rings, discoloration and fruiting structures) or vascular wilt (gradual wilting of the above ground shoots). Other signs are the presence of mycelium and fruiting bodies which range in size from microscopic to easily detectable with the eye. They are found within the leaf spot or stem rot area. The skilled person knows other forms of damages which are caused by fungi on plants. The extent of damages can e.g. be determined by determining the relative area of spots on the plant leaves.

The term “KRE5 and/or KRE6” means either KRE5 or KRE6 or both of KRE5 and KRE6. Thus, the term “(a) KRE5 and/or KRE6 gene(s)” or “(a) KRE5 and/or KRE6 RNA(s)” or similar terms means either a KRE5 gene or a KRE6 gene or both of the KRE5 and KRE6 genes or a KRE5 RNA or a KRE6 RNA or both of the KRE5 and KRE6 RNAs. Consequently, “a DNA capable of expressing an inhibitory molecule” or “an inhibitory molecule” may comprise the nucleic acid against the KRE5 gene or the KRE6 gene or both of the KRE5 and KRE6 genes or against the KRE5 RNA or the KRE6 RNA or both of the KRE5 and KRE6 RNAs. Consequently, the plant of the present invention incorporates a DNA molecule or inhibitory nucleic acid or antisense sequence against the KRE5 gene or the KRE6 gene or both of the KRE5 and KRE6 genes or against the KRE5 RNA or the KRE6 RNA or both of the KRE5 and KRE6 RNAs. The terms “KRE5” and “kre5” and the terms “KRE6” and “kre6” are interchangeably used.

The term “expression of (a) KRE5 and/or KRE6 gene(s)” means (1) the transcription of (a) KRE5 and/or KRE6 gene(s) into (a) KRE5 and/or KRE6 RNA(s) and/or (2) the translation of (a) KRE5 and/or KRE6 RNA(s) into (a) KRE5 protein and/or KRE6 protein(s).

As used herein, an “antisense RNA” or “aRNA” is a single-stranded RNA which comprises an antisense sequence which is substantially complementary to (the) KRE5 and/or KRE6 mRNA(s) which is (are) transcribed in the fungus. The aRNA binds to KRE5 and/or KRE6 mRNA(s) produced by the fungus by base-pairing, thereby obstructing the translation machinery and inhibiting translation and further processing. Consequently, the production of KRE5 and/or KRE6 protein(s) is inhibited.

As used herein, an “RNA molecule in antisense direction” is an RNA molecule which comprises an antisense sequence.

As used herein, an “RNA molecule in sense direction” is an RNA molecule which comprises a sense sequence. An RNA molecule in antisense direction and an RNA molecule in sense direction may be one RNA molecule, i.e the antisense and sense sequences are present on the same RNA molecule, or may be different RNA molecules, i.e the antisense and sense sequences are present on different RNA molecules.

As used herein, a “double-stranded RNA” or “dsRNA” is an RNA molecule that is partially or completely double stranded. The dsRNA may be formed by an RNA molecule in antisense direction and an RNA molecule in sense direction. Double-stranded RNA may be formed by a single nucleic acid strand which comprises the sense and antisense sequences. In this case, the RNA molecule in sense direction and the RNA molecule in antisense are on the same RNA molecule. The resulting dsRNA has a hairpin or stem-loop structure, whereby the stem is formed by the sense and anti-sense sequences. The loop may be formed by a sequence which has no substantial complementarity within the strand and lies between the sense and antisense sequences. Such a loop sequence can be an intron sequence of a gene, such as the intron of the RGA2 gene of wheat (Loutre et al., 2009, Plant Journal 60, 1043-1054) or the second intron of the Cut2 gene of M. oryzae.

Alternatively, the dsRNA may be formed by two different RNA strands, whereby the antisense sequence and the sense sequence are on different strands. In this case, the RNA molecule in antisense direction is different from the RNA molecule in sense direction.

The term “dsRNA” comprises dsRNA which is capable of activating the RNAi pathway in a plant cell. The term “dsRNA” also comprises small or short interfering RNA (siRNA) and micro-RNA (miRNA). The two latter dsRNAs are either expressed in a transgenic plant of the present invention from the DNA capable of expressing an inhibitory nucleic acid molecule or are produced by the RNAi machinery in the plant cell starting from a longer dsRNA expressed from the DNA. The term “dsRNA” also comprises circular interfering RNA (ciRNA), short hairpin RNA (shRNA) and the like. The term “dsRNA” also includes dsRNA, such as hpRNA, siRNA, miRNA or longer dsRNA, comprising antisense and sense sequences, which is not produced within a transgenic plant, but is produced by genetic engineering or synthesizing methods in the laboratory and which is for external application on a plant and/or fungus.

As used herein, “RNAi” or “RNA interference” refers to the process of sequence-specific gene silencing, mediated by double-stranded RNA (dsRNA). In order to function, one strand of the dsRNA comprises an antisense sequence which is substantially complementary to (the) KRE5 and/or KRE6 mRNA(s) or (a) part(s) thereof which is (are) transcribed in the fungus. The other strand comprises a sense sequence which is substantially complementary to the antisense sequence. The dsRNA is capable of activating the RNAi pathway in a plant cell resulting in the formation of a single stranded RNA comprising the antisense strand which binds to (the) KRE5 and/or KRE6 mRNA(s) in the fungus and results in the degradation of the mRNA.

According to the present invention, the sizes of the sense or antisense sequence or the sizes of the RNA molecule in sense direction or the RNA molecule in antisense direction may be the same. Alternatively, the sizes may differ.

The DNA capable of expressing an inhibitory nucleic acid molecule results, in one embodiment of the present invention, upon expression in the plant cell in an mRNA molecule which may function as an aRNA and inhibit KRE5 and/or KRE6 mRNA(s) in the fungus. Such DNA molecule comprises on the sense strand an antisense sequence.

In another embodiment, the DNA which is capable of expressing an inhibitory nucleic acid molecule results upon expression in the plant cell in an mRNA molecule or in mRNA molecules which may form a dsRNA which may activate the RNAi machinery. Within this embodiment, the DNA encodes an RNA molecule in sense direction and an RNA molecule in antisense direction. The sense sequence and the antisense sequence may be present on one DNA strand. This may result in an mRNA molecule which comprises the sense sequence and antisense sequence on the same mRNA molecule. Due to the reverse substantial complementarity of the sense sequence and antisense sequence, a hairpin dsRNA will form. Alternatively, if e.g. the sense and antisense sequences being present on the same DNA strand are under the control of different promoters or of one bidirectional promoter, different mRNA molecules are transcribed one harboring the sense sequence and the other harboring the antisense sequence. Different mRNA molecules, one harboring the sense sequence and the other harboring the antisense sequence, may also form if the sense sequence and the antisense sequence are present on different strands of the DNA. Due to the invers substantial complementarity of the sense and antisense sequences, the mRNAs form a dsRNA. The dsRNA may be recognized as foreign by the plant cell and may activate the RNAi machinery. The dsRNA transcribed from the DNA may be an siRNA or miRNA molecule or may be processed to an siRNA or miRNA molecule. After infection of the plant with a fungus, an exchange of the RNA generated in the plant may occur between the plant and the fungus. The RNA incorporated in the fungus may lead to a sequence-specific gene silencing of the KRE5 and/or KRE6 gene(s). It is known that the siRNA effect can be continued in plants if the RNA dependent RNA polymerase synthesizes new siRNAs from the degraded mRNA fragments. These secondary or transitive RNAis can enhance the silencing.

As used herein, the term “DNA”, “DNA molecule” or DNA polynucleotide” etc. refers to double-stranded DNA, unless otherwise specified.

As used herein, “substantially complementary” refers to polynucleotide strands that are capable of base pairing according to the standard Watson-Crick complementarity rules. It is understood that two polynucleotides hybridize to each other even if they are not completely complementary to each other. Consequently, the term “substantially complementary” means that two polynucleotide sequences are complementary over at least 80% of their nucleotides, preferably over at least 85%, 90%, 95%, 96%, 97%, 98%, 99%. Most preferably, the two polynucleotide sequences are complementary over 100% of their nucleotides. Alternatively, “substantially complementary” means that two polynucleotide sequences can hybridize under high stringency conditions. It is understood that an antisense sequence which is substantially complementary to (a) fungal KRE5 and/or KRE6 gene(s) or transcript(s) has a low degree of complementarity to functionally identical or similar genes in plants, preferably below 60%, and does not hybridize or only to a low degree to endogenous plant genes or transcripts, so that the transcription and/or translation of plant genes is not reduced or reduced to at most 10%, as compared to a non-transgenic plant of identical phenotype.

As used herein, the term “hybridize(s)(ing)” refers to the formation of a hybrid between two nucleic acid molecules via base-pairing of complementary nucleotides. The term “hybridize(s)(ing) under stringent conditions” means hybridization under specific conditions. An example of such conditions includes conditions under which a substantially complementary strand, namely a strand composed of a nucleotide sequence having at least 80% complementarity, hybridizes to a given strand, while a less complementary strand does not hybridize. Alternatively, such conditions refer to specific hybridizing conditions of sodium salt concentration, temperature and washing conditions. As an example, highly stringent conditions comprise incubation at 42° C., 50% formamide, 5×SSC (150 mM NaCl, 15 mM trisodium citrate), 50 mM sodium phosphate, 5×Denhardt's solution, 10×dextran sulphate, 20 mg/ml sheared salmon sperm DNA and washing in 0.2×SSC at about 65° C. (SSC stands for 0.15 M sodium chloride and 0.015 M trisodium citrate buffer). Alternatively, highly stringent conditions may mean hybridization at 68° C. in 0.25 M sodium phosphate, pH 7.2, 7% SDDS, 1 mM EDTA and 1% BSA for 16 hours and washing twice with 2×SSC and 0.1% SDDs at 68° C. Further alternatively, highly stringent hybridisation conditions are, for example: Hybridizing in 4×SSC at 65° C. and then multiple washing in 0.1×SSC at 65° C. for a total of approximately 1 hour, or hybridizing at 68° C. in 0.25 M sodium phosphate, pH 7.2, 7% SDS, 1 mM EDTA and 1% BSA for 16 hours and subsequent washing twice with 2×SSC and 0.1% SDS at 68° C.

For introducing the DNA molecule capable of expressing an inhibitory nucleic acid molecule into a plant or a part thereof, the DNA molecule or the expression cassette harboring the DNA may be inserted into a vector. Vectors which harbor a DNA polynucleotide for effecting inhibition of gene expression in a plant cell are known to those in the art and are also useful for the purposes of the present invention of inhibiting the expression of (a) KRE5 and/or KRE6 gene(s) in a fungus. In addition to the DNA molecule, the vector may comprise heterologous regulatory element(s) in the 5′ and optionally in the 3′ positions which are able to function in a plant. The vector may comprise a promoter regulatory sequence that is functional in plant cells, operably linked to the DNA molecule, and optionally a terminator regulatory sequence. Such a vector may be a hairpin vector or a double promoter vector, as known in the art. An exemplary double promoter vector is disclosed in FIG. 20. Using this type of vector, the inserted DNA molecule is transcribed bidirectionally. An inverted double promoter allows the expression of one DNA sequence in the 3′ direction and of a second DNA sequence in the 5′ direction, whereby the resulting RNAs are substantially complementary to each other and generate dsRNA. Alternatively, two promoters or a bidirectional promoter, e.g. the mannopine synthase promoter (Guevara-Garcia et al., 1993, Plant Journal (3):495-505), may be employed such that one promoter regulates the transcription of a DNA sequence comprising an antisense sequence and the second promoter regulates the transcription of a DNA sequence comprising an antisense sequence, which, however, is present on the DNA not in complementary location to the sense sequence. Suitable binary vectors for the transformation of plants are the pBINPLUS vector (van Engelen et al., 1995, Transgenic Research 4, 288-290), the pGPTV vector (Becker et al., 1992, Plant Mol. Biol., 29, 1195-1197) , the p6U and p7U vector (DNA Cloning Service e. K., Hamburg, Germany; www.dna-cloning.com; U.S. Pat. No. 7,834,243).

According to the invention, the term “promoter regulatory sequence” or “promoter” is intended to mean any promoter of a gene that can be expressed in a plant. Such promoter may be a promoter which is naturally expressed in a plant or is of bacterial or viral origin. Preferably, the promoter is a tissue specific promoter and/or a pathogen inducible promoter. Examples of promoters of plant origin are the histone promoter (EP 0 507 698) or the rice actin promoter (U.S. Pat. No. 5,641,876). Examples of promoters of a plant virus gene are the cauliflower mosaic virus (CaMV 19S or 35S), the cassava vein mosaic virus (CsVMV: WO97/48819) or the circovirus promoter (AU 689 311). Examples of tissue-specific promoters are the napin (EP 255 378), phaseolin, glutenin, helianthinin (WO 9217580), albumin (WO 9845460) and oleosin (WO 9845461) promoter. Examples of inducible promoters are the promoters of phenylalanine ammonia lyase (PAL), of HMG-CoA reductase (HMG), of chitinases, of glucanases, of proteinase inhibitors (PI), of genes of the PR1 family, of nopaline synthase (nos) or of the vspB gene (U.S. Pat. No. 5,670,349), the HMG2 promoter (U.S. Pat. No. 5,670,349), the apple beta-galactosidase (ABG1) promoter or the apple amino cyclopropane carboxylate synthase (ACC synthase) promoter (WO 98/45445), or chimeric pathogen inducible promoters (WO 00/29592; WO 2007/147395; WO 2013/091612).

According to the invention, the term “terminator regulatory sequence” is intended to mean any such sequence that is functional in a plant, also comprising polyadenylation sequences. It may be of bacterial origin such as the nos or ocs terminator of Agrobacterium tumefaciens, of viral origin such as the CaMV 35S terminator, or of plant origin such as a histone terminator as described in EP 0 633 317.

The selection step for identifying a transformed plant or a part thereof comprising the DNA molecule or a processed construct can be carried out via a selectable gene present in the vector, as referred to above. The selectable gene may comprise an operably linked promoter regulatory sequence and terminator regulatory sequence that are functional in plant cells.

Among the selectable markers that can be used in the present invention, reference is made to genes for resistance to antibiotics, such as the hygromycin phosphotransferase gene, the neomycin phosphotransferase II gene inducing resistance to kanamycin, or the aminoglycoside 3″-adenyltransferase gene, genes for tolerance to herbicides such as the bar gene (White et al., Nucl. Acids Res., 1990, 18: 1062) for tolerance to bialaphos, the EPSPS gene (U.S. Pat. No. 5,188,642) for tolerance to glyphosate or else the HPPD gene (WO 96/38567) for tolerance to isoxazoles, genes encoding identifiable enzymes, such as the GUS enzyme, GFP protein or genes encoding pigments or enzymes regulating pigment production in the transformed cells. Such selectable marker genes are in particular described in patent applications WO 91/02071, WO 95/06128, WO 96/38567, and WO 97/04103.

Marker gene free transformation is another alternative to transfer the expression cassette of interest into the plant.

For introducing the DNA molecule into a plant or a part thereof, numerous methods are known in the art. A preferred method applied in the present invention is transformation of the DNA molecule, expression cassette or vector harboring the DNA molecule by the use of bacteria of the Agrobacterium genus, preferably by infection of the cells or tissues of plants with A. tumefaciens (Knopf, 1979, Subcell. Biochem. 6: 143-173; Shaw et al., 1983, Gene 23(3): 315-330) or A. rhizogenes (Bevan and Chilton, 1982, Annu. Rev. Genet. 16: 357-384; Tepfer and Casse-Delbart, 1987, Microbiol. Sci. 4(1): 24-28). For example, the transformation of plant cells or tissues with Agrobacterium tumefaciens is carried out according to the protocol described by Hiei et al. (1994, Plant J. 6(2): 271-282). Another preferred method is the biolistic transformation method, wherein cells or tissues are bombarded with particles onto which the vectors of the invention are adsorbed (Bruce et al., 1989, Proc. Natl. Acad. Sci. USA 86(24): 9692-9696; Klein et al., 1992, Biotechnology 10(3): 286-291; U.S. Pat. No. 4,945,050). A further method is the widely used protoplast transformation. Therefor, plant cells are separated by pectinases and subsequently, the cell wall is degraded to generate protoplasts. For transformation, polyethylene glycol is added or electroporation is applied. Other methods are bringing the plant cells or tissues into contact with polyethylene glycol (PEG) and the vectors of the invention (Chang and Cohen, 1979, Mol. Gen. Genet. 168(1): 111-115; Mercenier and Chassy, 1988, Biochimie 70(4): 503-517). Electroporation is another method, which consists in subjecting the cells or tissues to be transformed and the vectors of the invention to an electric field (Andreason and Evans, 1988, Biotechniques 6(7): 650-660; Shigekawa and Dower, 1989, Aust. J. Biotechnol. 3(1): 56-62). Another method consists in directly injecting the vectors into the cells or the tissues by microinjection (Gordon and Ruddle, 1985, Gene 33(2): 121-136). Those skilled in the art will choose the appropriate method according to the nature of the plant to be transformed and the fungus against which the plant is to be rendered resistant.

Cells or tissues of plants, e.g., root cells grown in culture, can be transformed with the desired gene and grown into mature plants. When transformation is effective, the transgene will be incorporated into the pollen and eggs and passed on to the next generation.

In one embodiment, the DNA molecule or expression cassette is stably integrated into the genome of the transgenic plant, preferably into a chromosome of the plant. Integration can, however, also occur into an extrachromosomal element. By stable integration into the genome of a plant, the DNA sequences can be passed to subsequent generations of the transgenic plant. Alternatively, the DNA molecule or expression cassette is present within the plant cell on the vector used to introduce the DNA molecule and is not stably integrated into the genome of the plant. Therefore, the DNA sequences may not be passed to subsequent generations of the plant.

According to the invention, all plants and all parts of a plant can be treated according to the methods of the present invention. By plants is meant all plants and plant populations such as desirable and undesirable wild plants, cultivars and plant varieties (whether or not protectable by plant variety or plant breeder's rights). Cultivars and plant varieties can be plants obtained by conventional propagation and breeding methods which can be assisted or supplemented by one or more biotechnological methods such as by use of double haploids, protoplast fusion, random or directed mutagenesis, molecular or genetic markers or by bioengineering or genetic engineering methods.

The term “part of a plant” refers to any parts or organs of a plant such shoot vegetative organs/structures, e.g., leaves, stems or tubers; roots, flowers or floral organs/structures, e.g. bracts, sepals, petals, stamens, carpels, anthers or ovules; seed, including embryo, endosperm or seed coat; fruit or the mature ovary; plant tissue, e.g. vascular tissue or ground tissue; or cells, e.g. guard cells, egg cells or trichomes; or progeny of the same. The term “cell” refers to a cell or cell accumulation within the plant as well as to an isolated cell or isolated cell accumulation. A cell may have a cell wall or may be a protoplast. In particular, the present invention relates to a seed which comprises the DNA, expression cassette or vector as comprised by the present invention. Preferably, the seeds of a transgenic plant retain the DNA, expression cassette or vector as comprised by the invention, so that the new plants generated from a seed continues to comprise the DNA, expression cassette or vector,

Plants that can be protected by the method according to the invention comprise all plants, preferably plants of economic interest, more preferably the plant according to the present invention is selected from the group consisting of barley (Hordeum vulgare), sorghum (Sorghum bicolor), rye (Secale cereale), Triticale, sugar cane (Saccharum officinarium), maize (Zea mays), foxtail millet (Setaria italic), rice (Oryza sativa), Oryza minuta, Oryza australiensis, Oryza alta, wheat (Triticum aestivum), Triticum durum, Hordeum bulbosum, purple false brome (Brachypodium distachyon), sea barley (Hordeum marinum), goat grass (Aegilops tauschii), apple (Malus domestica), strawberry, sugar beet (Beta vulgaris), sunflower (Helianthus annuus), Australian carrot (Daucus glochidiatus), American wild carrot (Daucus pusillus), Daucus muricatus, carrot (Daucus carota), eucalyptus (Eucalyptus grandis), Erythranthe guttata, Genlisea aurea, woodland tobacco (Nicotiana sylvestris), tobacco (Nicotiana tabacum), Nicotiana tomentosiformis, tomato (Solanum lycopersicum), potato (Solanum tuberosum), coffee (Coffea canephora), grape vine (Vitis vinifera), cucumber (Cucumis sativus), mulberry (Morus notabilis), thale cress (Arabidopsis thaliana), Arabidopsis lyrata, sand rock-cress (Arabidopsis arenosa), Crucihimalaya himalaica, Crucihimalaya wallichii, wavy bittercress (Cardamine flexuosa), peppergrass (Lepidium virginicum), sheperd's-purse (Capsella bursa-pastoris), Olmarabidopsis pumila, hairy rockcress (Arabis hirsuta), rape (Brassica napus), broccoli (Brassica oleracea), Brassica rapa, Brassica juncacea, black mustard (Brassica nigra), radish (Raphanus sativus), Eruca vesicaria sativa, orange (Citrus sinensis), Jatropha curcas, cotton (Gossipium sp.), soybean (Glycine max), and black cottonwood (Populus trichocarpa). Particularly preferred, the plant is selected from the group consisting of barley (Hordeum vulgare), sorghum (Sorghum bicolor), rye (Secale cereale), Triticale, sugar cane (Saccharum officinarium), maize (Zea mays), rice (Oryza sativa), wheat (Triticum aestivum), Triticum durum, Avena sativa, Hordeum bulbosum, sugar beet (Beta vulgaris), sunflower (Helianthus annuus), carrot (Daucus carota), tobacco (Nicotiana tabacum), tomato (Solanum lycopersicum), potato (Solanum tuberosum), coffee (Coffea canephora), grape vine (Vitis vinifera), cucumber (Cucumis sativus), thale cress (Arabidopsis thaliana), rape (Brassica napus), broccoli (Brassica oleracea), Brassica rapa, Brassica juncacea, black mustard (Brassica nigra), radish (Raphanus sativus), cotton (Gossipium sp.) and soy-bean (Glycine max).

The invention concerns a method of producing a transgenic plant or a part thereof, comprising the steps of introducing into at least a cell of the plant the DNA or the expression cassette or the vector as comprised by the invention, and regenerating the transgenic plant from the at least one cell.

As used herein “regenerating” means a process of growing an entire plant from a single cell, a group of cells, a part of the plant or a tissue of the plant. The skilled person knows methods of introducing DNA into at least a cell of the plant and growing a plant therefrom. “At least a cell” means a single cell, a group of cells, a part of the plant or a tissue of the plant.

Furthermore, the invention concerns a method of conferring fungal resistance to a plant or a part thereof comprising the steps of introducing into the plant or the part thereof the DNA or the expression cassette or the vector as comprised by the invention, and causing expression of the DNA or the expression cassette.

“As used herein, the term “causing expression” means that under the conditions, under which the plant is kept and/or cultivated, transcription of the DNA having been introduced into the plant is induced. For example, if the promoter is an inducible promoter, the activity of such promoter can be induced by the presence or absence of specific biotic or abiotic factors, according to the choice of the user of the present invention. If the promoter is a constitutive promoter, expression continuously occurs.

Furthermore, the invention concerns a method of inhibiting the expression of the KRE5 and/or KRE6 gene(s) in a fungus, comprising applying the DNA or the expression cassette or the vector as comprised by the invention to the fungus or to a plant or a part thereof.

As used herein “applying . . . to the fungus or to a plant or a part thereof” or a similar term means that the DNA, expression cassette or vector as comprised by the invention are administered to the fungus, plant or part thereof so that the fungus, plant or part thereof incorporate and express the DNA. The application on the plant may be in the laboratory using any methods of introducing the DNA, expression cassette or vector, as referred to above, into the plant or the part thereof. The application on the plant may also be in the field in order to render the plant which may be infected by a phyto-pathogenic fungus resistant to the fungus. The application may also be directly on the fungus in order to cause cessation of infection, growth, development, reproduction and/or pathogenicity and eventually death of the fungus. “Directly” means that the DNA, expression cassette or vector is applied on the fungus, so that the DNA, expression cassette or vector is introduced into the fungus without plant involvement. The fungus may have already infected the plant or may still be present outside the plant such as in the soil. The application in the field may occur by any method known in the art of applying a fungicide, such as spraying or splashing.

Furthermore, the invention concerns the use of the DNA or the expression cassette or the vector as comprised by the invention for inactivating a fungus, while contacting a plant or a part thereof; for protecting a plant against an infection by a fungus; or for inhibiting the expression of the KRE5 and/or KRE6 gene(s) in a fungus.

As used herein, “inactivating a fungus” means causing cessation of infection, growth, development, reproduction and/or pathogenicity and eventually death of the fungus. Inactivation occurs by RNA molecules directed against the KRE5 and/or KRE6 gene(s) or mRNA(s) of the fungus produced by the transgenic plants. Alternatively, inactivation occurs by applying the DNA, expression cassette or vector as comprised by the present invention onto the fungus outside the plant, such in the field. Inactivation of the fungus is obtained by reduction of the expression of the KRE5 and/or KRE6 gene(s) or mRNA(s) of the fungus, resulting in the reduction of the damages of the plant.

As used herein, “while contacting a plant” means that the fungus is in touch with the plant. Thus, the fungus is forming or has already formed structures to invade into cell of the plant. This means that differentiated structures of the fungus such as an appresorium is forming or has already formed and is penetrating or has already penetrated into a cell of the plant.

As used herein, “protecting a plant” means conferring resistance to the fungus on the plant. A resistant plant is not infested and/or damaged by a fungus or is infested and/or damaged by a fungus to a lower extent as compared to a plant of the same phenotype which is not treated according to the present invention. By introducing a DNA capable of expressing an inhibitory nucleic acid into a plant or a part thereof, resistance to a fungus harboring the KRE5 and/or KRE6 gene(s), against which the inhibitory molecule is directed, is obtained, so that the plant is protected from the fungus. To determine the resistance, the transgenic plant may be compared with a control plant which has the identical phenotype, however, does not contain the transgene. Resistance can be determined using an optical score wherein scores from non-resistant, if the symptoms of the transgenic plant correspond to those of the non-transgenic plant, to highly resistant, if no symptoms of fungus infection are seen, may be awarded. Alternatively, resistance can be determined by determining the KRE5 and/or KRE6 transcript amount(s) in the fungus during infection of a transgenic plant, as compared to the transcript amounts of the fungus of identical phenotype infected on a non-transgenic plant of identical phenotype, or in the fungus of same phenotype which does not infect a plant. The present inventors have found that, dependent on the amount of transcript reduction, sporulation of the fungus is reduced, the adhesion and formation of functional infection structures is prevented and the vegetative growth of the hyphae is strongly delayed. Reduction of the KRE5 and KRE6 transcript amounts by 47% and 49%, respectively, was sufficient to have a visibly negative effect on necrotrophic hyphae. Thus, transcript reduction of at least 40%, 50%, 60%, 70%, 80%, 90%, 95% or of 100% for the KRE5 transcript and/or transcript reduction of at least 40%, 50%, 60%, 70%, 80%, 90%, 95% or of 100% for the KRE6 transcript allows scoring the transgenic plant as being resistant or as having resistance to a fungus. Preferably, the damages on the plant are reduced by at least 50%, 60%, 70%, 80%, 90% or by 100% in a resistant transgenic plant according to the present invention as compared to a non-transgenic plant of identical phenotype.

The invention also concerns a composition comprising the DNA molecule capable of expressing inhibitory nucleic acid molecules capable of inhibiting the expression of (a) KRE5 and/or KRE6 gene(s) in a fungus, a composition comprising an expression cassette comprising the DNA molecule, a composition comprising a vector comprising the DNA molecule or a composition comprising dsRNA capable of inhibiting the expression of (a) KRE5 and/or KRE6 gene(s) in a fungus. The composition may be for external application on a plant and/or a fungus.

Alternatively, the composition may contain dsRNA, wherein one strand of this RNA comprises an antisense sequence which is substantially complementary the KRE5 and/or KRE6 gene(s) or mRNA(s) and the other strand comprises a sense sequence which is substantially complementary to the antisense sequence. The dsRNA may be an hpRNA, miRNA or siRNA. The definitions of dsRNA, sense and antisense sequences are given above. When the composition is applied to a plant and/or to a fungus from the outside, such as in the field, the dsRNA may be incorporated into the plant and/or the fungus and may inhibit the expression of (a) KRE5 and/or KRE6 gene(s) or mRNA(s) in the fungus via gene silencing such as via the antisense mechanism or the RNAi mechanism.

Double-stranded RNA for the manufacture of the composition in accordance with the invention can be produced in vitro using methods known to the skilled person. Thus, the dsRNA may be synthesized by genetic engineering methods from a DNA or by synthesis methods.

The composition in accordance with the invention may be used as a fungicide for a plant or a part thereof which is already infected by a fungus or will be potentially infected. The composition may also be used against the fungus, which has already infected the plant or which has not infected the plant, for example when the fungus is still outside the plant, for example in the soil. In this regard, the composition is used to control the growth of the pathogenic fungus either by the treatment of a plant or by the application on the fungus. The skilled person knows fungicide compositions and knows which further ingredients such as carrier substrates, whereby the carrier substrate has, for example, an RNA-stabilizing effect, to include into the composition and what methods to use in order to prepare and apply the composition to a plant and/or fungus in the field. The composition in accordance with the invention may furthermore be used as a pre-treatment for seed.

The invention is further explained in the following figures as examples, which are included for illustration purposes and are not intended to limit the invention.

### FIGURES

FIG. 1A and B: The phylogenetic trees indicate close relatedness of KRE5 and KRE6 of C. graminicola with corresponding KRE genes of filamentous fungi and yeasts. Filamentous Ascomycota are framed and labelled with ,A′, yeasts is framed and labelled with ,B′, Basidiomycota is framed and labelled with ,C′.

FIG. 2: Relationship of KRE5 proteins of filamentous fungi. Filamentous Ascomycota are labelled with ,A′, yeasts labelled with ,B′, Basidiomycota labelled with ,C′. The secretion signal (small bars); UDP-glucose:glycoprotein glucosyltransferase domain (striped bars); and glycosyl-transferase family 8-like domain (black bars) of Kre5 are shown. ER retention signal is given in capital letters. Protein sizes are indicated in amino acids (aa).

FIG. 3: Structure and size of KRE6 proteins of filamentous fungi and yeasts. Filamentous Ascomycota are labelled with ,A′, yeasts labelled with ,B′, Basidiomycota labelled with ,C′. Transmembrane domains (small bars) and family 16-like glycohydrolase 16 domains (large bars) of Kre6 are shown. Protein sizes are indicated in amino acids (aa).

FIG. 4: Alignments of the KRE5 protein sequences of various fungi of the ascomycotes and basidiomycotes Consensus denotes the consensus sequence based on the KRE 5 proteins as indicated (Consensus sequence KRE5; SEQ ID NO: 372).

FIG. 5: Alignments of the KRE6 protein sequences of various fungi of (A) the ascomycotes and (B) basidiomycotes. Consensus denotes the consensus sequence based on the KRE 6 proteins as indicated ((A) Consensus sequence KRE6 ascomycotes; SEQ ID NO: 373; (B) Consensus sequence KRE6 basidiomycotes; SEQ ID NO: 374).

FIG. 6. Complementation of Saccharomyces Δkre5 and Δkre6 mutants by Colletotrichum graminicola KRE5 and KRE6 cDNAs.

(A) Growth of dilution series of yeast strains on different solidified substrata. WT, S. cerevisiae reference strain Y00000; Δkre5 and Δkre6, KRE5 and KRE6-deficient S. cerevisiae strains Y21633 and Y05574 TpAG300, Δkre5 and Δkre6 transformant harboring the empty binary vector pAG300; TKRE51-4, independent Δkre5 transformants expressing the KRE5 cDNA of C. graminicola; TKRE61-4, independent Δkre6 transformants expressing the KRE6 cDNA of C. graminicola. YPD, yeast extract peptone dextrose agar; YPDS, YPD agar supplemented with sorbitol; or YPDS Calcofluor and YPDS Killer toxin K1, YPDS containing Calcofluor White or Killer Toxin K1. Number of yeast cells inoculated were (left to right) 5×10⁴, 5×10³, 5×10², and 5×10. Cells were grown at 30° C. for two days and photographed.

(B) Gowth of yeast strains in liquid YPDS. Names of strains are as described in (A). Arrowhead in insert of micrograph showing Δkre6 indicates filament emerging from yeast cell. Bars are 5 μm.

FIG. 7. Generation and characterization of KRE5:mCherry and KRE6:mCherry replacement strains.
(A) Scheme of generation of KRES5:mCherry and KRE6:mCherry replacement strains by homologous recombination. PoliC, oliC promoter; Nat-1, nourseothricin acetyl transferase-1 gene. Bars indicate position of probes used in Southern blot experiments.
(B) Genomic Southern blot showing that the 4.1 kb WT band has been replaced by a 7.5 kbmCherry fusion band in two independent replacement strains.
(C) OMA plates colonized by the WT and two independent KRES5:mCherry and KRE6:mCherry replacement strains. Plates were photographed 14 DAI.
(D) Growth rates of WT and two independent KRE5:mCherry and KRE6:mCherry replacement strains.
(E) Number of conidia formed by the WT and two independent KRE5:mCherry and KRE6:mCherry replacement strains.
(F) Virulence assay with the WT and two independent KRES5:mCherry and KRE6:mCherry replacement strains on maize leaves. Similar symptom intensities indicate similar virulence.

FIG. 8. Localization of Kre5:mCherry, Kre6:mCherry, and Gls1:eGFP in vegetative hyphae and protoplasts released from hyphae after treatment with cell wall-degrading enzymes.

(A) Vegetative hypha viewed using differential interference microscopy (DIC) or fluorescence microscopy. Fluorescence shows localization of Kre5:mCherryand of Gls1:eGFP. The merged micrographs (merge) show distinct localization of Kre5 (arrows) and Gls1 (arrows), as indicated by occurrence of distinct vesicles with merged flourescence, and co-localization of Kre6 and Gls1 in macro-vesicles, as indicated by additional fluorescence. Bars are 10 μm.

(B) Protoplasts 6 H or 24 H after release from hyphae. Note distinct localization of Kre5 and Gls1 and co-localization of Kre6 and Gls1. Bars are 10 μm.

FIG. 9. Asexual sporulation defects in KRE5- and KRE6-RNAi strains.

(A) On oat meal agar (OMA) and on OMA supplemented with osmolytes such as KCL or sorbitol, all RNAi strains show strongly reduced spore numbers, as compared with the WT strain. Only those KRE5- and KRE6-RNAi strains showing more than 40% of the transcript concentration of the WT strain are able to form conidia.

(B-H) While the WT strain produces falcate conidia, KRE5- and KRE6-RNAi strains produce small and misshapen spores (C and F), form aggregates in conidia (D and G, arrows), or burst and release lipid droplets (E and H). Bars are 10 μm.

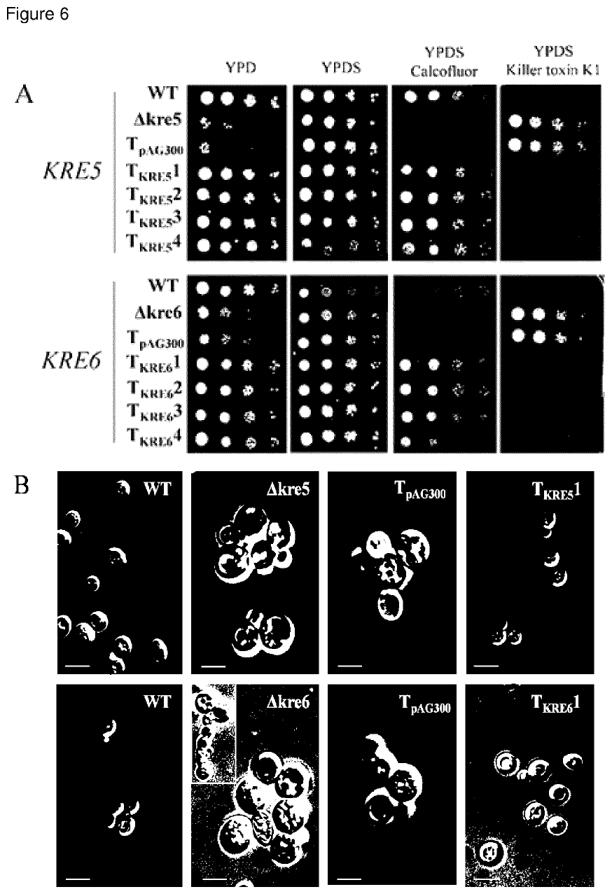

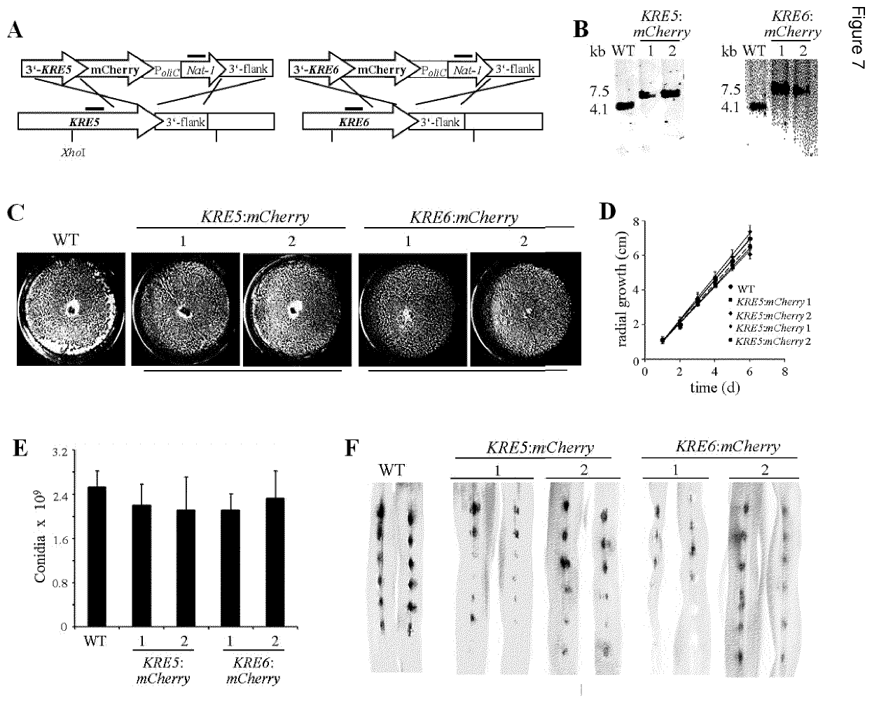

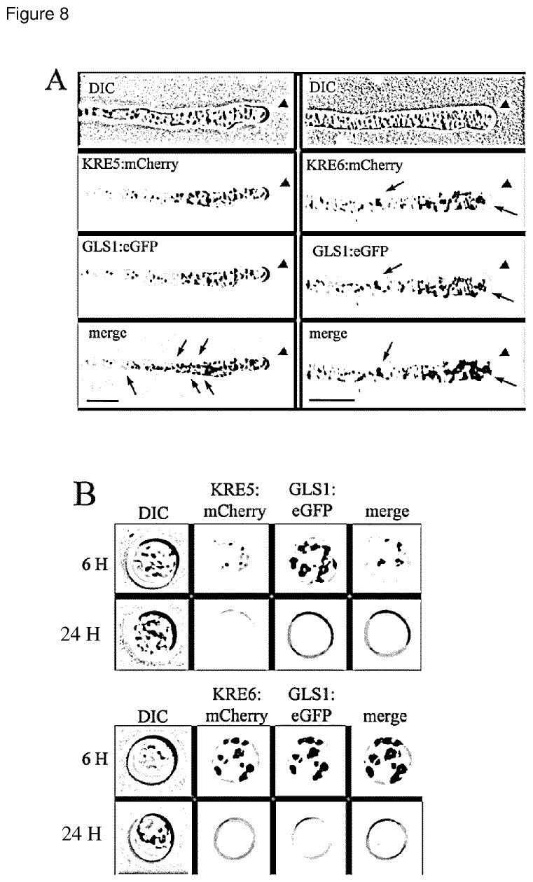

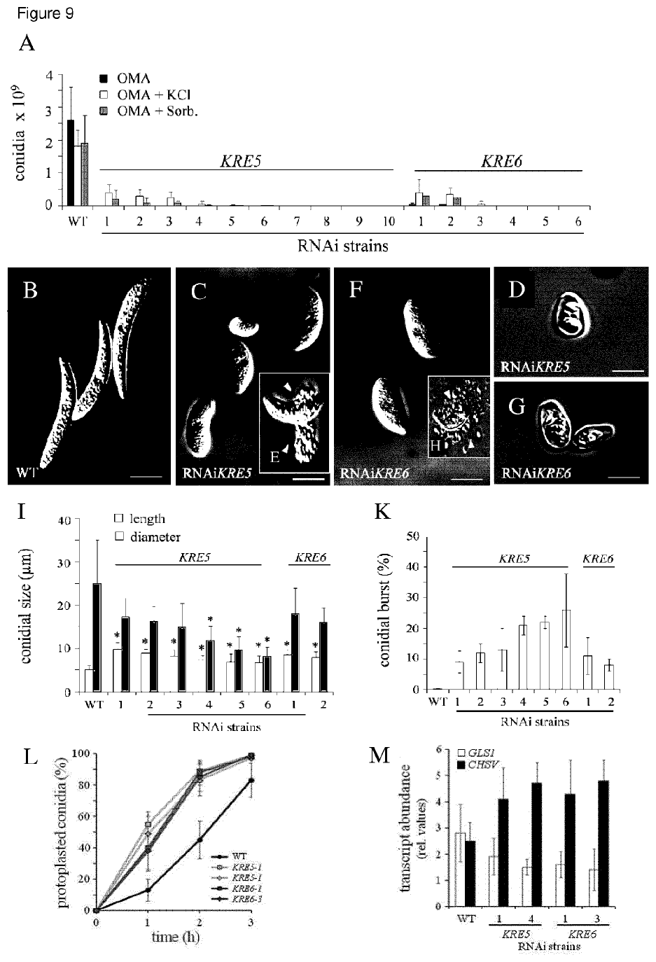

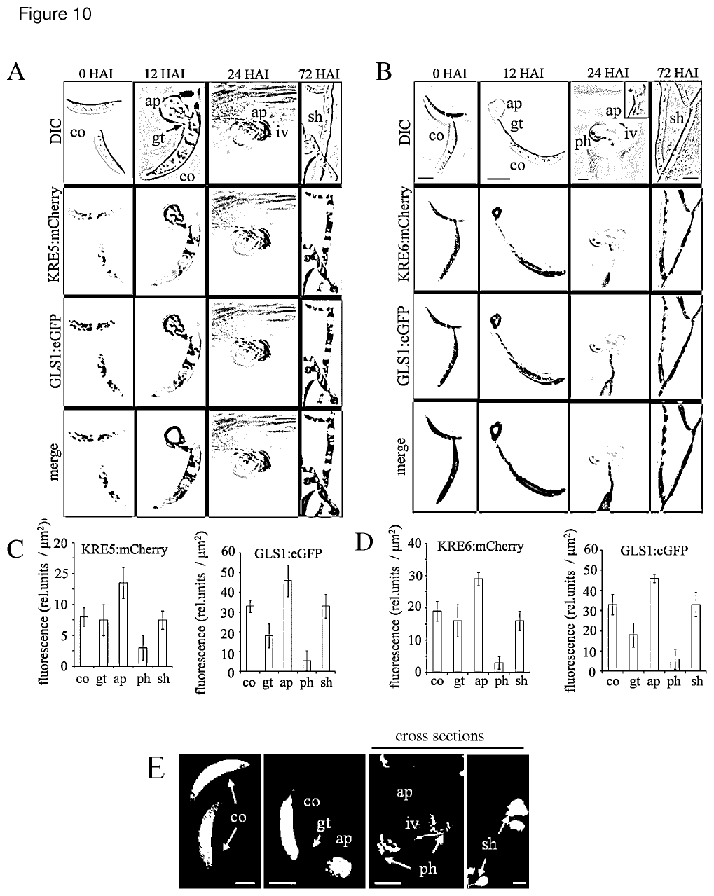

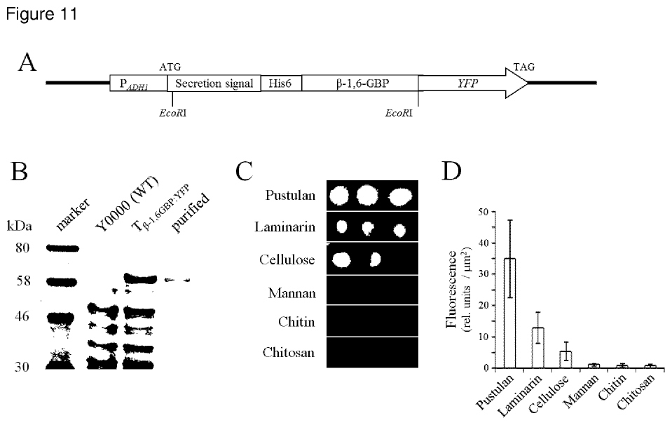

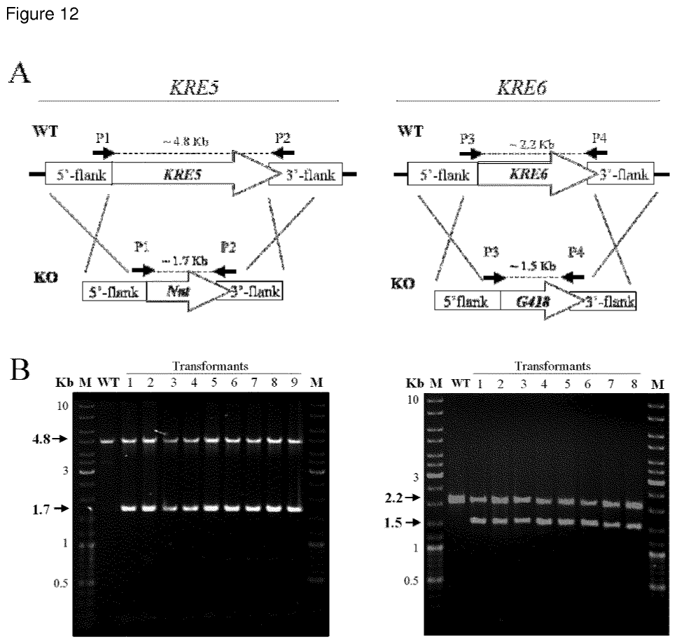

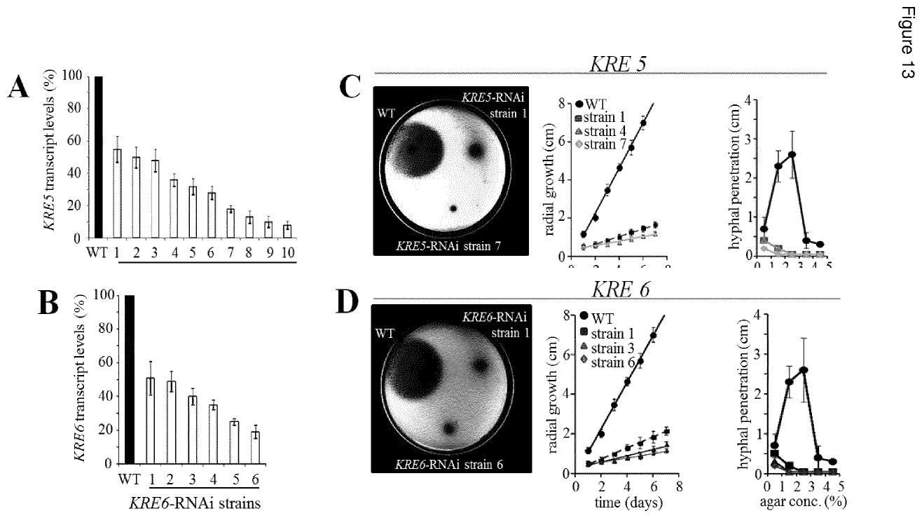

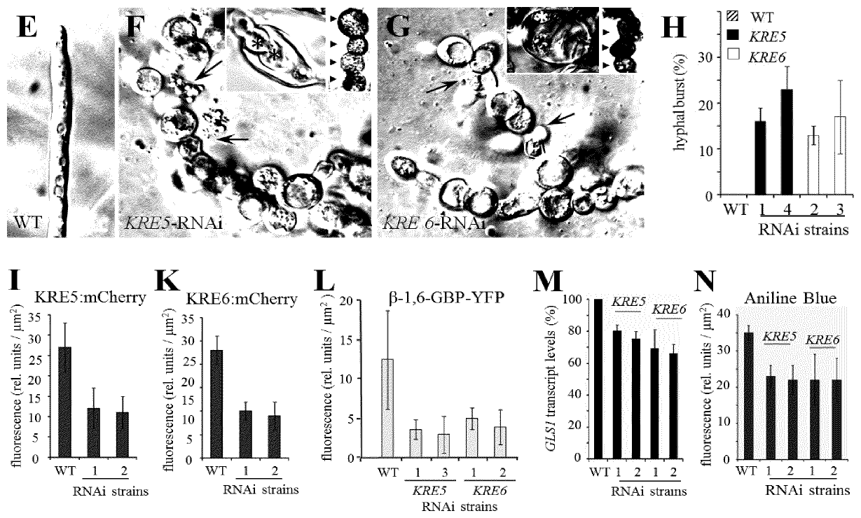

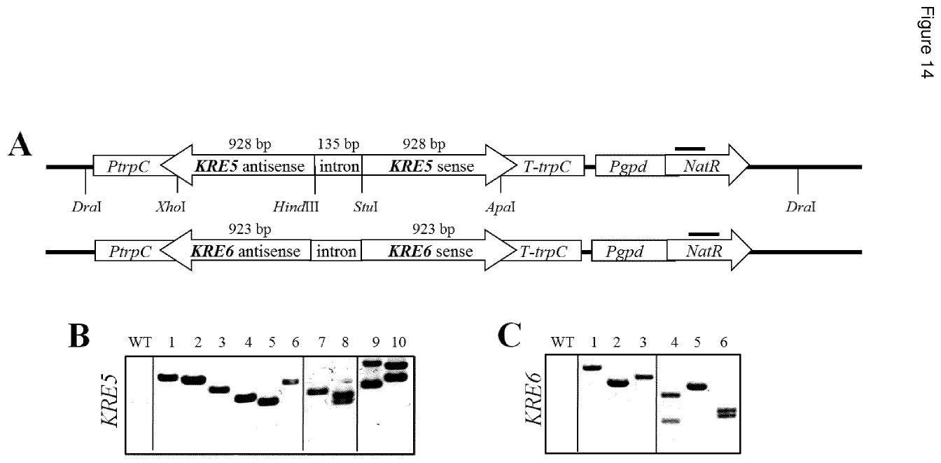

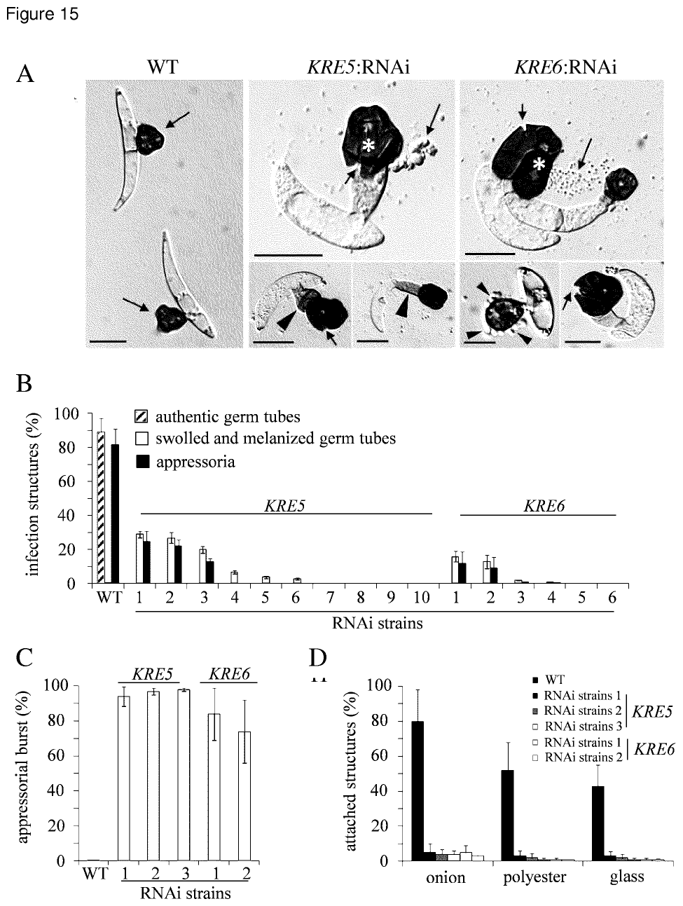

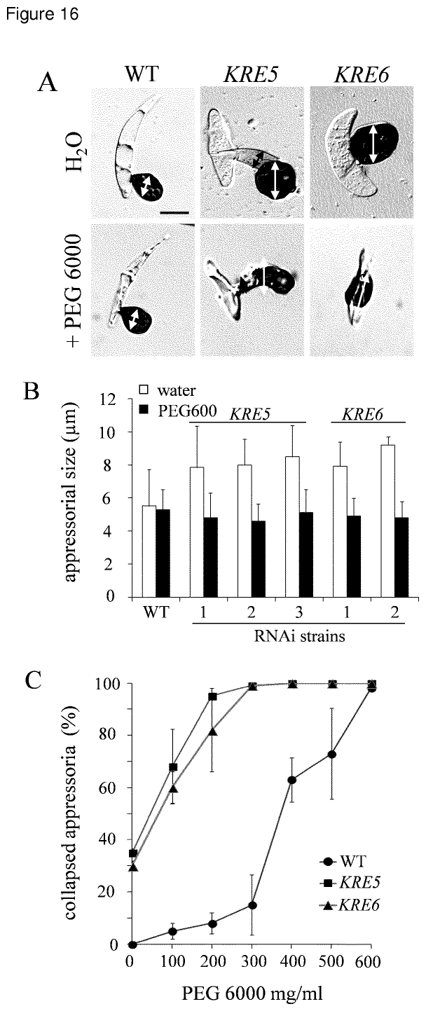

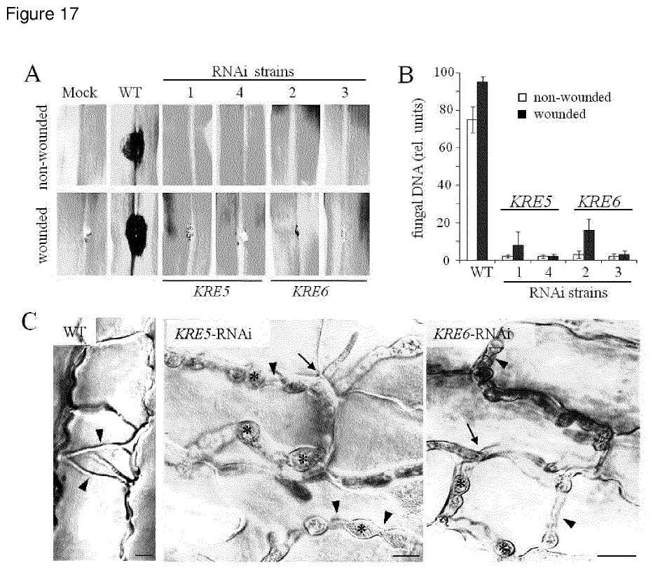

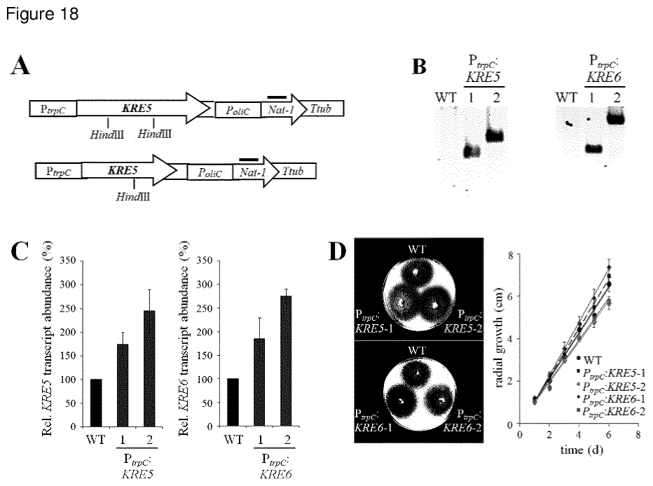

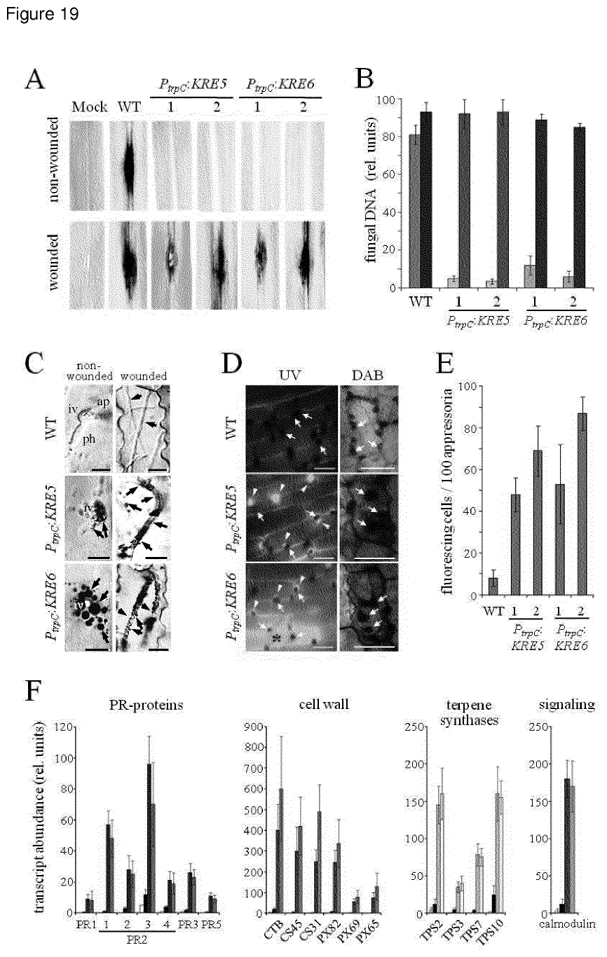

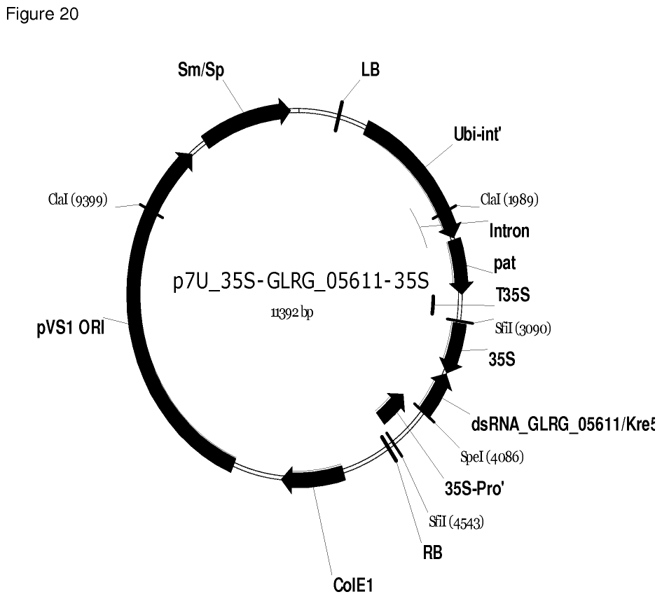

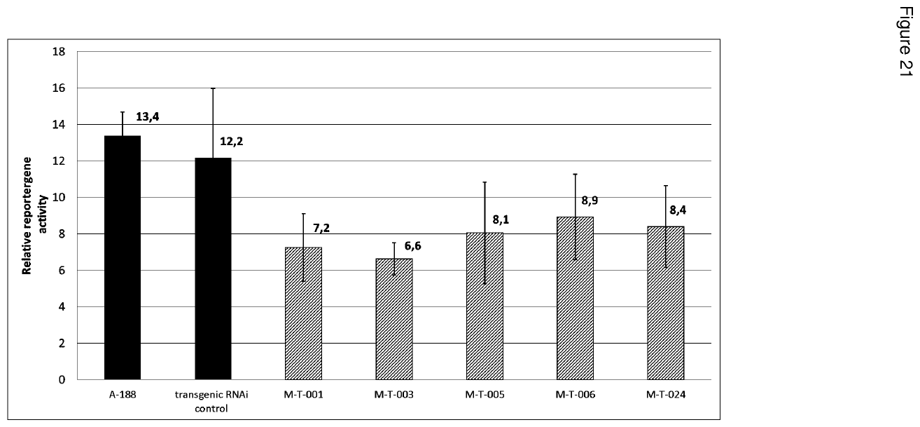

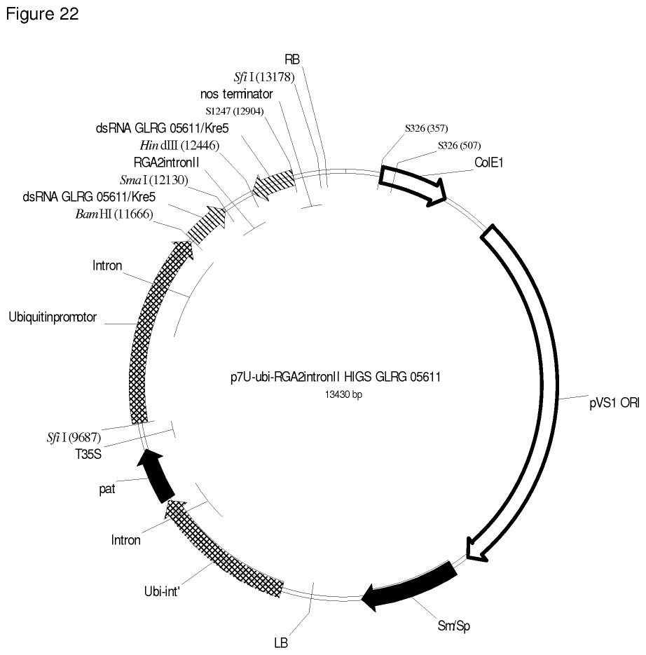

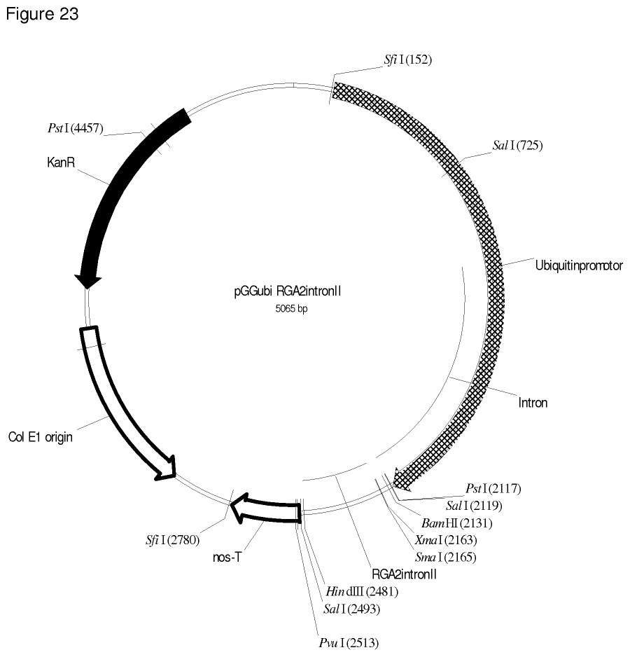

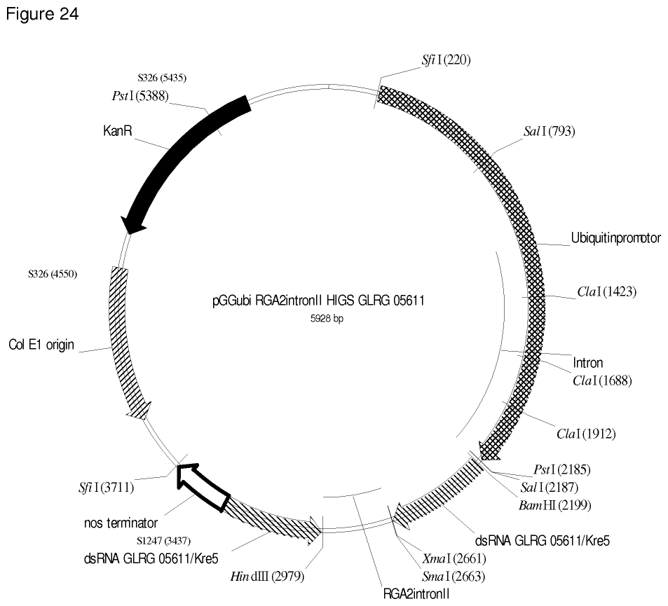

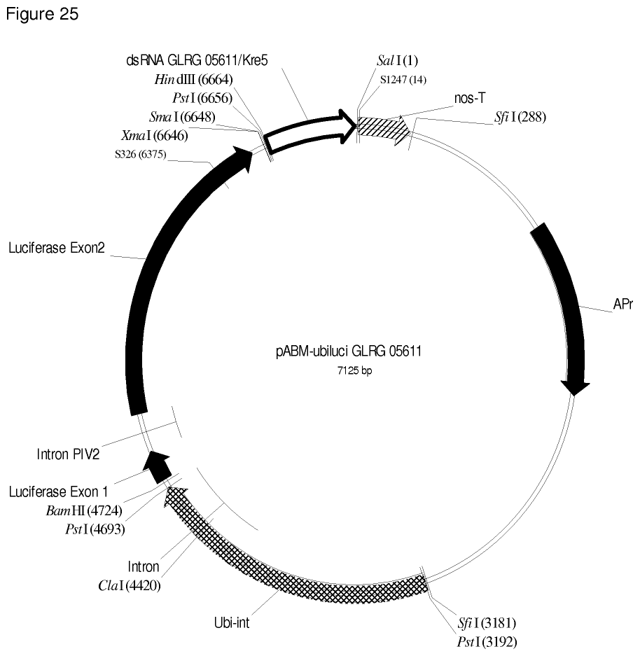

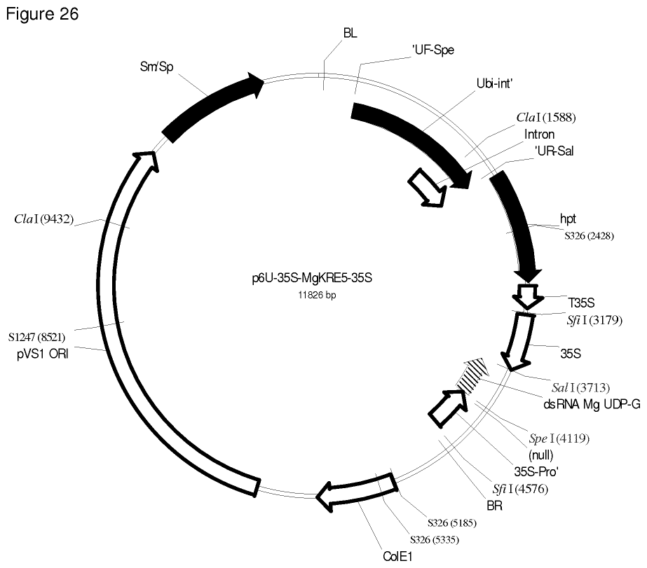

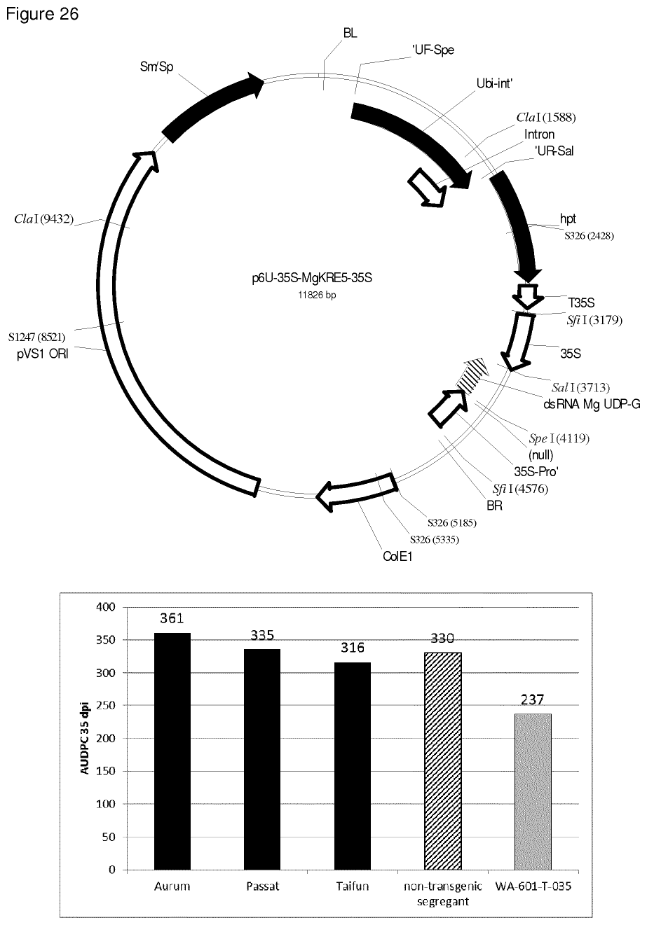

(I and K) Quantification of conidial size and fraction of conidia that burst spontaneously. Note that conidial length and rate of rupture correlates with KRE5 and KRE6 transcript abundance (see FIG. 6).

(L) Cell wall defects in KRE5- and KRE6-RNAi strains are evident, as oval conidia of these generate, when incubated in lysing enzymes, release protoplasts faster than the WT strain.

(M) Transcript abundance of GLS1, in contrast to CHSV, is clearly reduced in KRE5- and KRE6-RNAi strains, suggesting that GLS1, KRE5, and KRE6 are co-regulated. Bars in A and I-M are ±standard deviations.

FIG. 10. Infection structure-specific formation of Kre5:mCherry and Kre6:mCherry, and of the Gls1:eGFP fusion proteins. DIC, differential interference microscopy; Kre5:mCherry, Kre6:mCherry, and Gls1:eGFP show fluorescing protein in different infection structures. ap, appressorium; co, conidium; gt, germ tube; iv, infection vesicle; ph, primary hypha; sh, secondary hypha. Bars in A, B, and E are 10 μm.

(A) Co-expression of KRE5 and GLS1 in conidia, germ tubes, appressoria and secondary hyphae. No fluorescence is observed in infection vesicles formed at 24 HAI.

(B) Co-expression of KRE6 and GLS1 in conidia, germ tubes, appressoria and secondary hyphae. No fluorescence is visible in infection vesicles (24 HAI). Arrows indicate a fluorescing conidium on the cuticle.

(C and D) Relative fluorescence of infection structures. Bars are ±standard deviations.

(E) Staining of β-1,6-glucan of intact infection structures formed on the cuticle (left two micrographs, and of cross-sections of in planta differentiated biotrophic and necrotrophic hypae using the β-1,6-GBP:YFP probe. Note that β-1,6-glucan is exposed on surfaces of conidia, appressoria, and in walls of secondary hypae, but not in walls of infection vesicles and primary hyphae.

FIG. 11. Generation and characterization of a probe detecting β-1,6-glucosidic bonds.

(A) Structure of the yeast expression vector used to produce and secrete the β-1,6-glucan binding protein fused to YFP (β-1,6-GBP:YFP). PADH1, yeast alcohol dehydrogenase 1 promoter; His6, His6 tag; β-1,6-GBP, β-1,6-glucan binding protein of C. graminicola; YFP, Yellow Fluorescing Protein.

(B) Patterns of proteins secreted by the S. cerevisiae WT strainY0000 and by transformant Tβ-1,6-GBP:YFP harboring the expression vector shown in (A). The β-1,6-GBP:YFP protein sectered into the growth medium was purified by a single Ni-affinity purification step (purified).

(C) Specificity of the β-1,6-GBP:YFP protein as tested with different polymeric cell wall carbohydrates in a dot-blot experiment.

(D) Quantitative fluorescence of the β-1,6-GBP:YFP protein bound to the carbohydrates spotted onto the Nylon filter shown in (C).

FIG. 12. PCR analyses showing that all transformants tested integrated the KRE5 and KRE6 deletion constructs ectopically.

(A) Scheme of planned deletion experiments by homologous recombination. Nat-1, nourseothricin acetyl transferase 1 gene; G418, aminoglycoside 3′-phosphotransferase gene.

(B) C. graminicola WT strain showing the KRE5 and KRE6 bands only, and transformants showing both the bands indicative of the intact KRE5 and KRE6 genes as well as of the antibiotic resistance genes.

FIG. 13. Effect of reduction of KRE5 and KRE6 transcript abundance by RNAi on vegetative hyphae

(A and B) Relative KRE5 and KRE6 transcript abundance in the WT strain, ten KRE5- and six KRE6-RNAi strains. Bars are ±standard deviations.

(C and D) Growth rates and penetration competence of selected KRE5- and KRE6-RNAi strains. Bars are ±standard deviations.

(E-H) Hyphal integrity of the WT, KRE5- and KRE6-RNAi strains. While the WT strains develops intact hyphae (E), KRE5-RNAi strains show hyphal swellings and ruptured hyphae releasing lipid vesicles (F, arrow), formation of intrahyphal hyphae (F, left insert, asterisks), and strong pigmentation of swellings (F, right insert, arrowheads). KRE6-RNAi strains also show hyphal swellings (G), from which protoplast-like bodies are released (arrows).

Intrahyphal hyphae (G, left insert, asterisks), and strong pigmentation of swellings (G, right insert, arrowheads) also occur in KRE6-RNAi strains. (H) While WT hyphae did not rupture, bursting hyphae were often observed in RNAi strains. Bars in E, F, and G are 10 μm. Bars in H are ±standard deviations.

(I-N) Effect of expression of KRE5- and KRE6-RNAi constructs on KRE5-(I) and KRE6-mCherry fluorescence (K), exposure of β-1,6-glucan, as visualized by β-1,6-GBP:YFP staining (L), GLS1 transcript levels (M), and exposure of β-1,3-glucan, as visualized by Aniline Blue Fluorochrome staining (N). Bars are ±standard deviations.

FIG. 14. RNAi constructs used for down-regulation of KRE5 and KRE6 transcript abundance in C. graminicola.

(A) Structure of RNAi constructs. PtrpC and TtrpC, trpC promoter and terminator; PoliC, oliC promoter; Nat-1, nourseothricin acetyl transferase gene. Bars indicate position of probes used in genomic Southern blots (B).

(B) Genomic Southern blots of independent transformants showing the number of integrated KRE5- and KRE6-RNAi cassettes.

FIG. 15. Appressorium formation and function are severely compromised in KRE5- and KRE6-RNAi strains of C. graminicola.

(A) While the WT strain germinates and differentiates melanized appressoria (WT, arrows), appressoria (asterisks) of both RNAi strains rupture (KRE5- and KRE6-RNAi, short arrows) and release vesicles (KRE5- and KRE6-RNAi, long arrows). In KRE5-RNAi strains, germ tubes appear to be melanized (KRE5-RNAi, arrowheads), in KRE6-RNAi strains release of vesicles often occurred over the entire appressorial surface (KRE6-RNAi, arrowheads). Bars are 10 μm.

(B) Quantification of pre-penetration infection structures formed by the WT and the RNAi strains.

(C) In contrast to the WT strain, the vast majority of appressoria differentiated by KRE5- and

KRE6-RNAi strains explode spontaneously.

(D) On all substrata tested KRE5- and KRE6-RNAi strains show severely reduced adhesion. Bars in B-D are ±standard deviations.

FIG. 16. Appressorial cell wall elasticity is significantly increased in KRE5- and KRE6-RNAi strains, resulting in reduction of turgor pressure.

(A) Appressoria of KRE5- and KRE6-RNAi strains swell in H2O and shrink in hypertonic PEG6000 solutions (400 mg/mL). In contrast, the diameter of the WT appressoria are unaffected by the osmotic potential of the surrounding medium. Note the length of the constant double arrow. Bars are 10 μm.

(B) Appressorial size of the WT and RNAi strains. White columns indicate diameters of appressoria in H2O, black columns give diameters of appressoria in PEG6000 (400 mg/mL). Bars are standard deviations.

(C) Incipient cytorrhizis experiments indicate that the appressorial turgor pressure is dramatically reduced in both, KRE5- and KRE6-RNAi strains, as compared with the WT strain. Bars are ±standard deviations.

FIG. 17. KRE5 and KRE6 are required for pathogenicity of C. graminicola.

(A) Infection assays were performed with the WT and selected KRE5- and KRE6-RNAi strains on non-wounded and wounded leaf segments. Mock-inoculated leaf segments were also included. Leaves were photographed six DAI.

(B) Quantification of virulence of WT, KRE5- and KRE6-RNAi strains on non-wounded and wounded leaf segments by qPCR. Samples for DNA extraction were taken six DAI. Bars are ±standard deviations.

(C) The WT strain colonized wounded leaves and formed thin secondary hyphae (arrow) growing across the host cell. KRE5- and KRE6-RNAi strains preferentially grew in close contact with the host cell wall, with hyphae showing swellings (asterisks). Arrows indicate growth across the host cell wall. The arrowhead points to brown pigmented regions. Micrographs were taken six DAI. Bars are 20 μm.

FIG. 18. Construction of C. graminicola strains carrying an additional KRE5 or KRE6 copy controlled by the constitutive trpC promoter of A. nidulans, and characterization of overexpression strains.

(A) KRE5 and KRE6 over-expression constructs transformed into the C. graminicola WT strain. PtrpC and TtrpC, trpC promoter; PoliC, oliC promoter; Nat-1, nourseothricin acetyl transferase-1 gene; TtubB, tubB promoter. Bars indicate position of probes used in genomic Southern blots (B).

(B) Genomic Southern blot analyses of the WT strain and of independent transformants, each harboring a single copy of the KRE5- and KRE6-overexpression construct.

(C) KRE5 and KRE6 transcript abundance in the WT strain and in independent transformants, harboring a single copy of the KRE5- and KRE6-overexpression construct.

(D) Morphology of colonies and growth rates of WT and two independent KRE5- and KRE6-overexpression strains on OMA. Plates were photographed 5 DAI.

FIG. 19. Overexpression of KRE5 and KRE6 during the biotrophic phase induces defense responses and causes non-pathogenicity of C. graminicola.

(A) Infection assays were performed with the WT and selected PtrpC:KRE5 and PtrpC:KRE6 strains on non-wounded and wounded leaf segments. Mock-inoculated leaf segments were also included. Leaves were photographed six DAI.

(B) Quantification of virulence of WT, PtrpC:KRE5 and PtrpC:KRE6 strains on non-wounded (left column) and wounded leaf segments (right column) by qPCR. Samples for DNA extraction were taken six DAI. Bars are ±standard deviations.

(C) DIC-microscopy of infection sites on non-wounded and wounded leaves. On non-wounded leaves the WT strain formed appressoria and differentiated biotrophic infection vesicles and primary hyphae. In wounded leaves, thin, fast growing secondary hyphae developed (arrowheads). On non-wounded leaves, PtrpC:KRE5 and PtrpC:KRE6 strains formed appressoria, which invaded the host cell and formed infection vesicles. Invading hyphae were covered by dark pigmented vesicles (PtrpC:KRE5, non-wounded, arrows). The micrograph showing the infection vesicle of the PtrpC:KRE6 strain (PtrpC:KRE6, non-wounded, iv) shows delivery of numerous large vesicles (PtrpC:KRE6, non-wounded, arrows) to the infection site. While the WT strain formed secondary hyphae in wounded leaves (WT, wounded, arrow-heads) without a visible plant response. Hyphae of PtrpC:KRE5 and PtrpC:KRE6 strains formed hyphae (PtrpC:KRE5 and PtrpC:KRE6, wounded, arrowheads), but hyphae were densely covered by dark vesicles (PtrpC:KRE5 and PtrpC:KRE6, wounded, arrows). Bars are 20 μm. Micrographs were taken six DAI.

(D) Fluorescence microscopy under UV light and microscopical investigation of DAB-stained leaf segments, visualizing formation of H2O2 indicated that both PtrpC:KRE5 and PtrpC:KRE6 strains, but not the WT strain, elicited strong defense responses. All strains differentiated appressoria on the leaf surface (arrows), but only the appressoria of PtrpC:KRE5 and PtrpC:KRE6 strains were decorated by brightly fluorescing papillae (arrowheads). Occasionally whole cell fluorescence was observed (asterisk). DAB staining revealed massive H2O2 formation in leaves inoculated with PtrpC:KRE5 and PtrpC:KRE6 strains, as indicated by browning, but not in leaves invaded by the WT strain. Bars are 30 μm.

(E) Quantification of cells fluorescing in response to infection with the WT and selected PtrpC:KRE5 and PtrpC:KRE6strains. Bars are ±standard deviations.

(F) Transcriptional response of maize leaves to infection with the WT, PtrpC:KRE5 and PtrpC:KRE6 strains. Mock-inoculated leaves served as control. Bars are ±standard deviations.

FIG. 20. Example of a binary plant transformation vector for establishing KRE5_{RNAI} plants. A selected sequence of the KRE5 gene of C. colletotrichum is under the control of two conversely transcribing 35S promoters.

FIG. 21. Detection of transcriptional silencing activity against the fungal KRE5 gene of Colletotrichum graminicola in the transgenic corn lines M-T-001, M-T-003, M-T-005, M-T-006 and M-T-024. Leaves of the non-transgenic genotype A-188, a transgenic RNAi control and the 5 HIGS lines were transiently transformed with the reporter gene construct pABM_ubiluci_GLRG_05611.

FIG. 22. Map of the plant transformation vector p7U-ubi_RGA2intronII_HIGS_GLRG_05611 which was used to transform a hairpin construct with a sense and antisense fragment of the Kre5 gene of Colletotrichum graminicola into maize.

FIG. 23. Map of the cloning vector pGGubi_RGA2intronII which was used for the construction of the HIGS vector pGGubi_RGA2intronII_HIGS_GLRG_05611.

FIG. 24. Map of the HIGS vector pGGubi_RGA2intronII_HIGS_GLRG_05611 which was used for the construction of the plant transformation vector p7U-ubi_RGA2intronII_HIGS_GLRG_05611.

FIG. 25. Map of the reporter gene vector pABM_ubiluci_GLRG_05611 which was used for the determination of transcriptional silencing activity of transgenic plants.

FIG. 26. Map of the wheat transformation vector p6U-35S-MgKRE5-35. A 401 bp large fragment of the KRE5 gene of M. graminicola was inserted between the inverse transcribed 35S promoters of the construct. 35S=35S promoter, dsRNA MgUDP-G=sequence of the M. graminicola gene KRE5, hpt=hygromycin B resistance gene, Ubi-int=maize ubiquitin promoter, T35S=terminator of the 35S Cauliflower mosaic virus.

### EXAMPLES

Identification of KRE5 Proteins

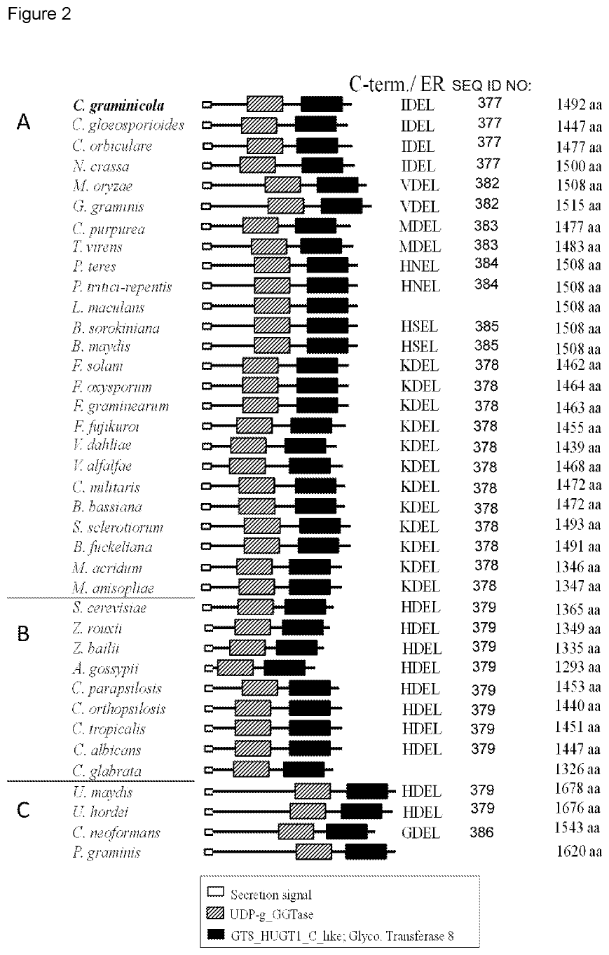

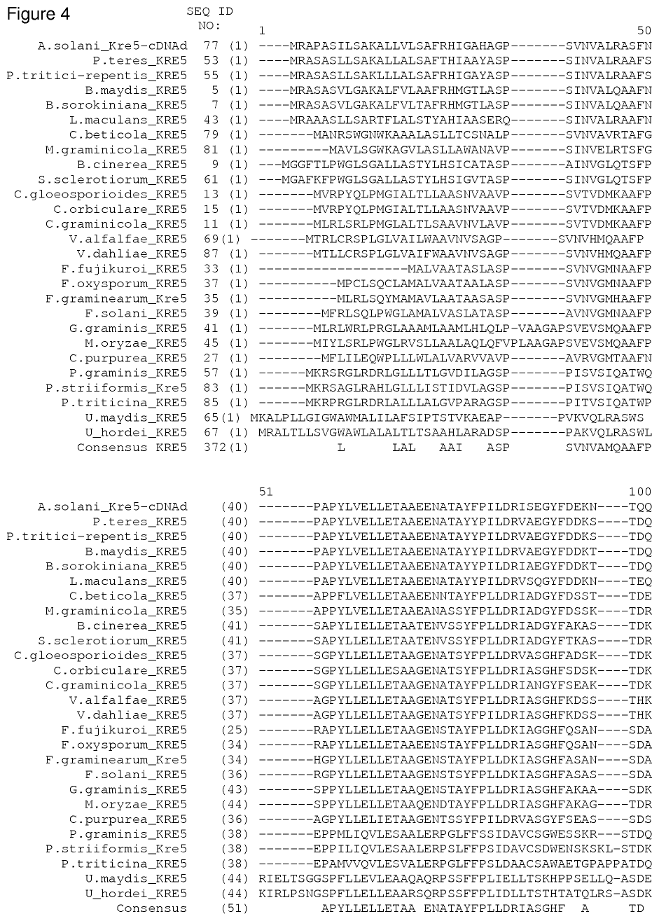

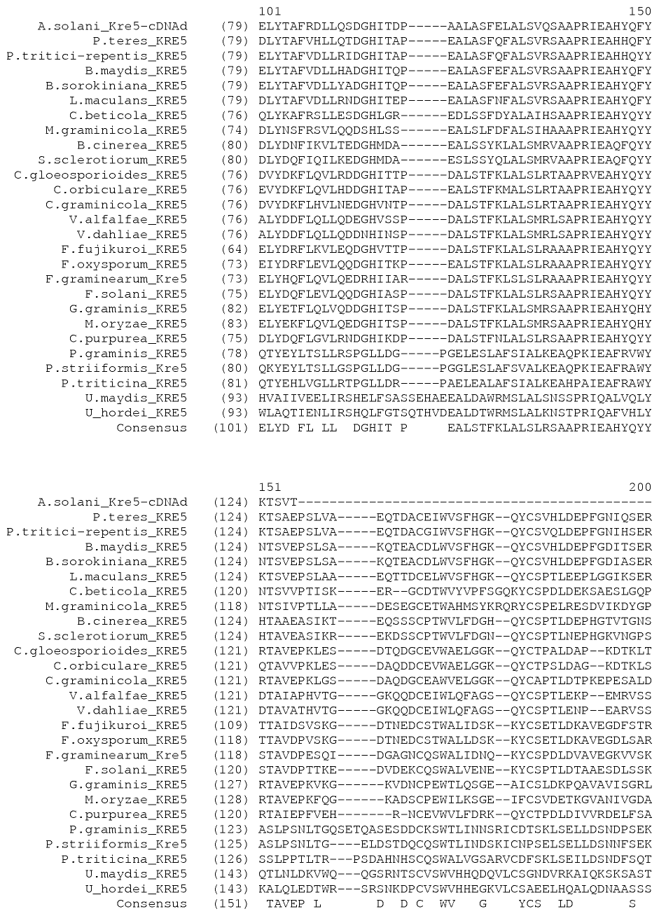

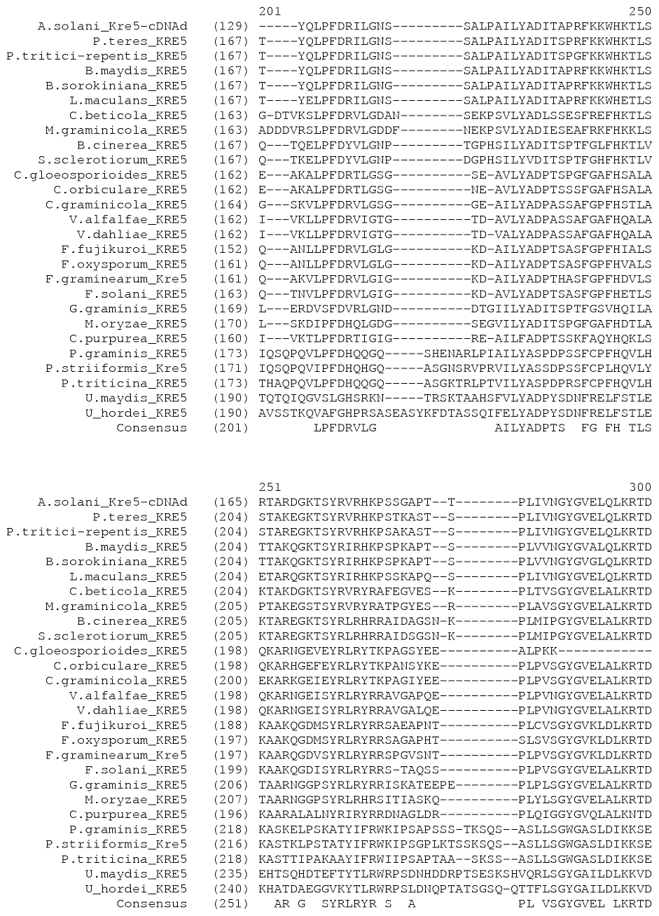

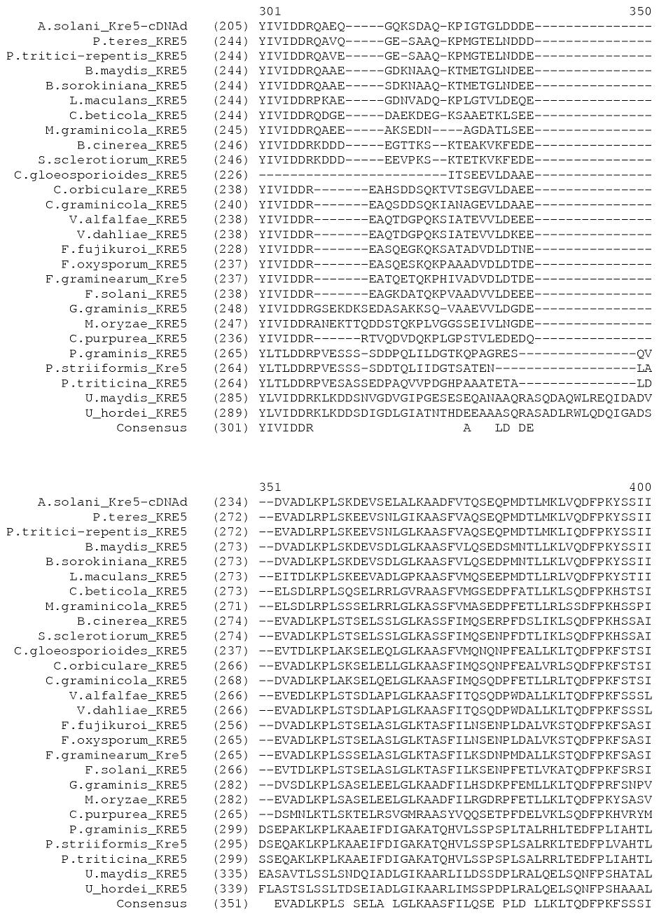

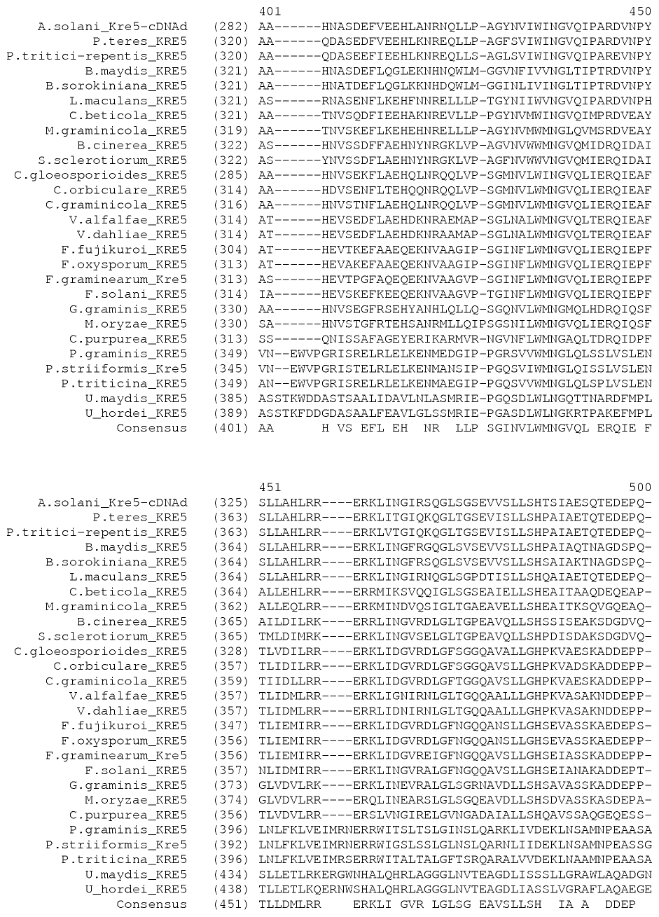

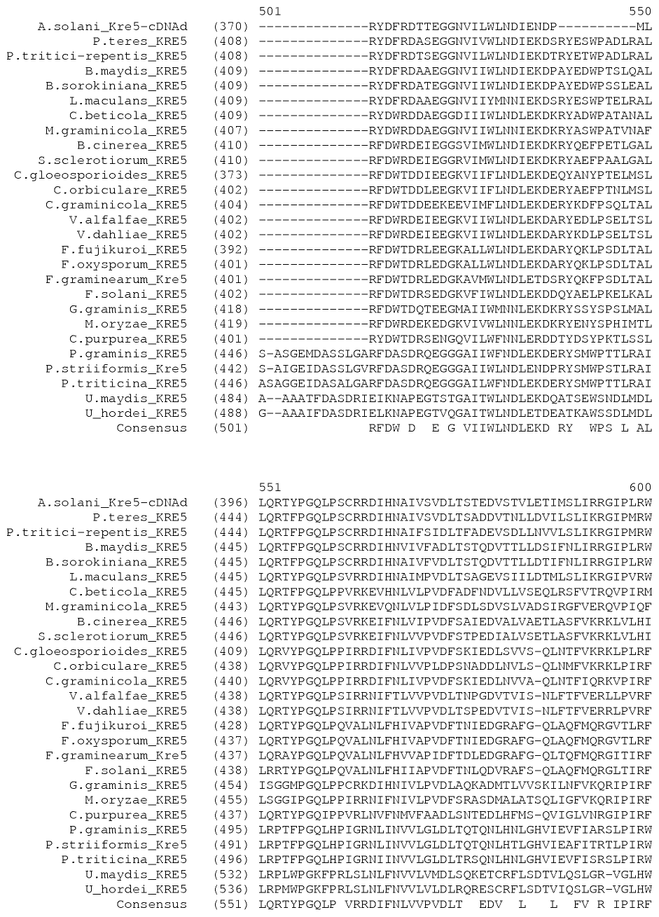

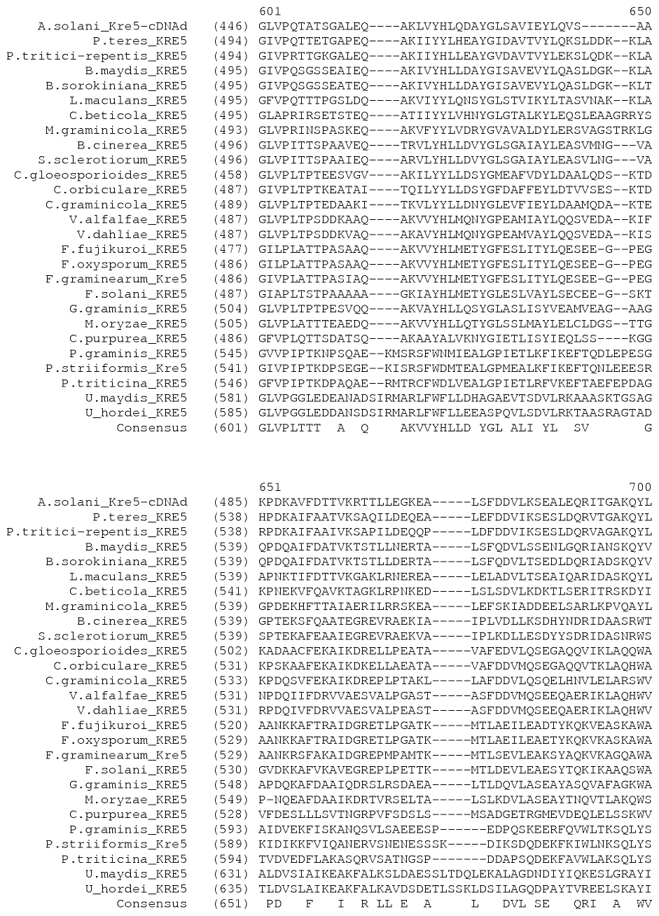

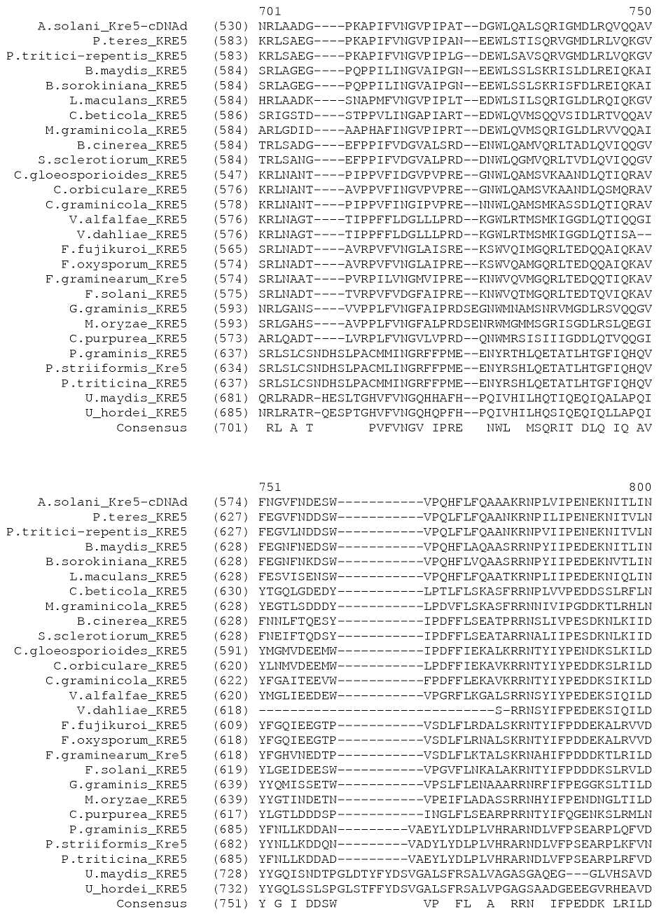

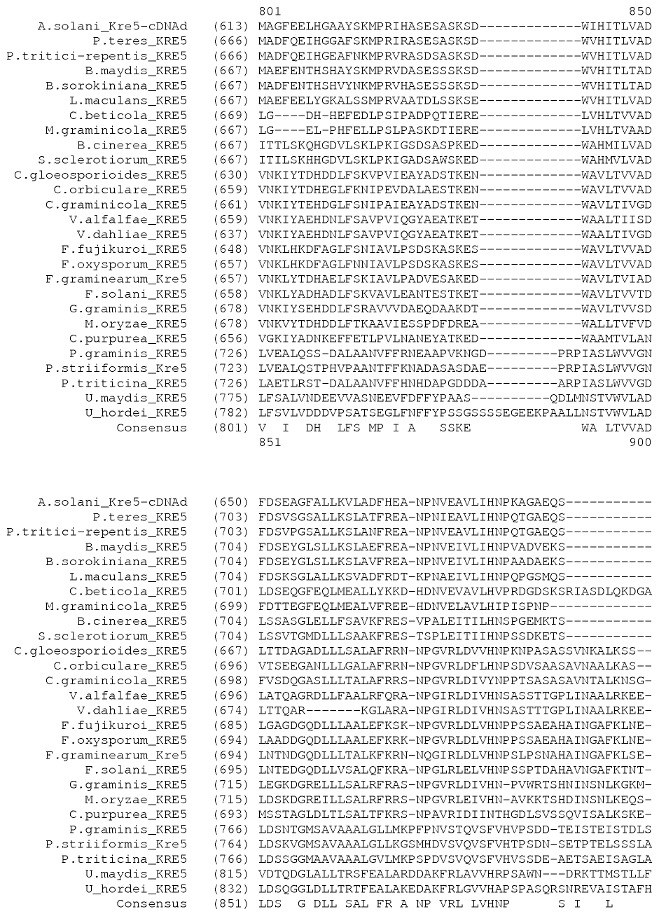

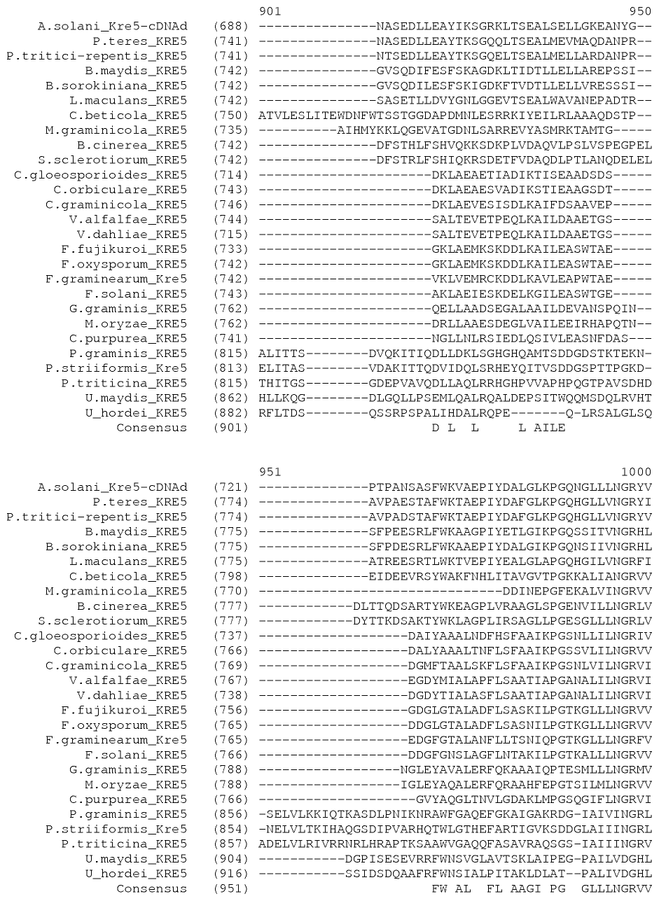

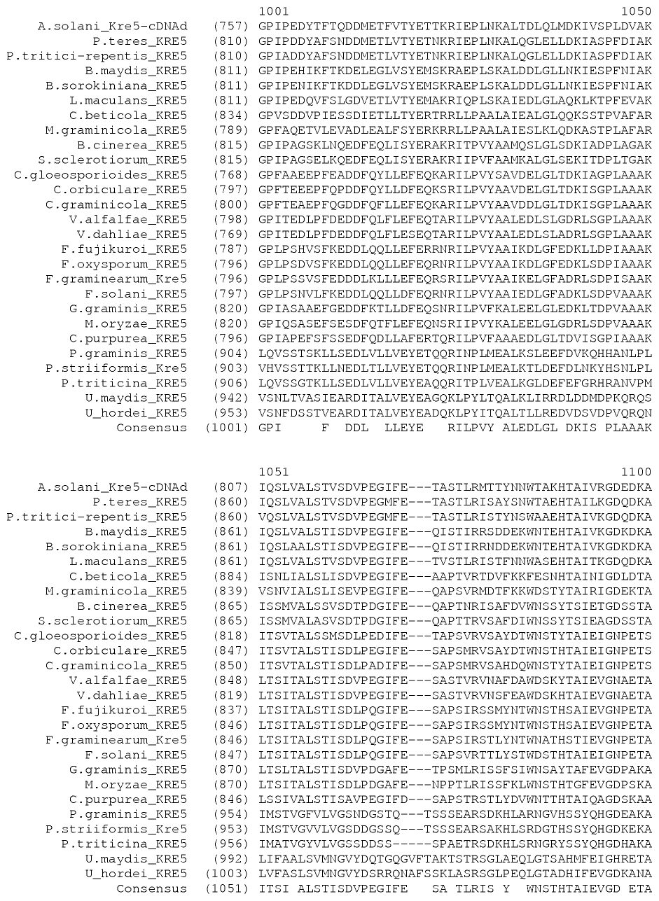

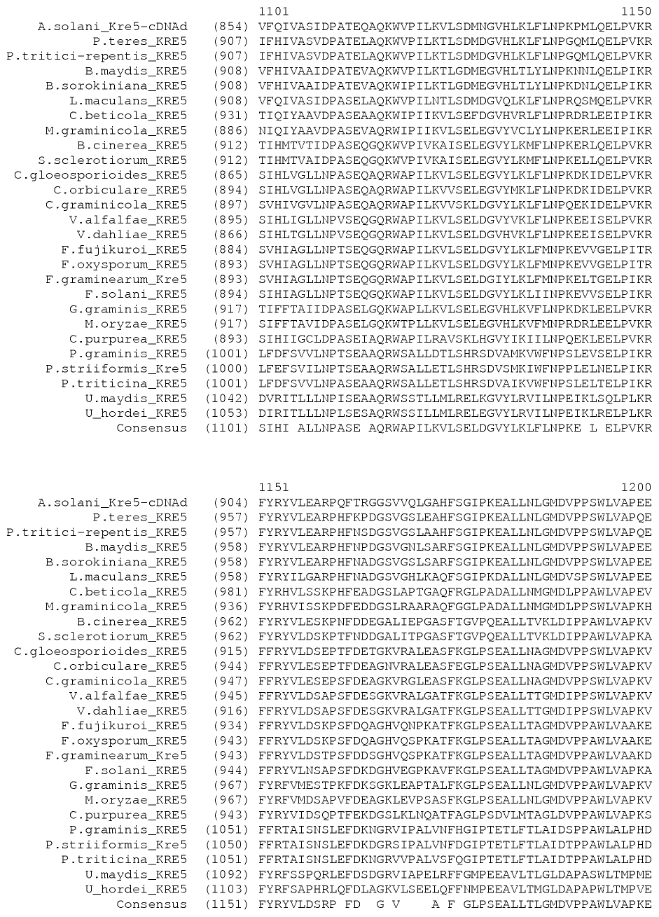

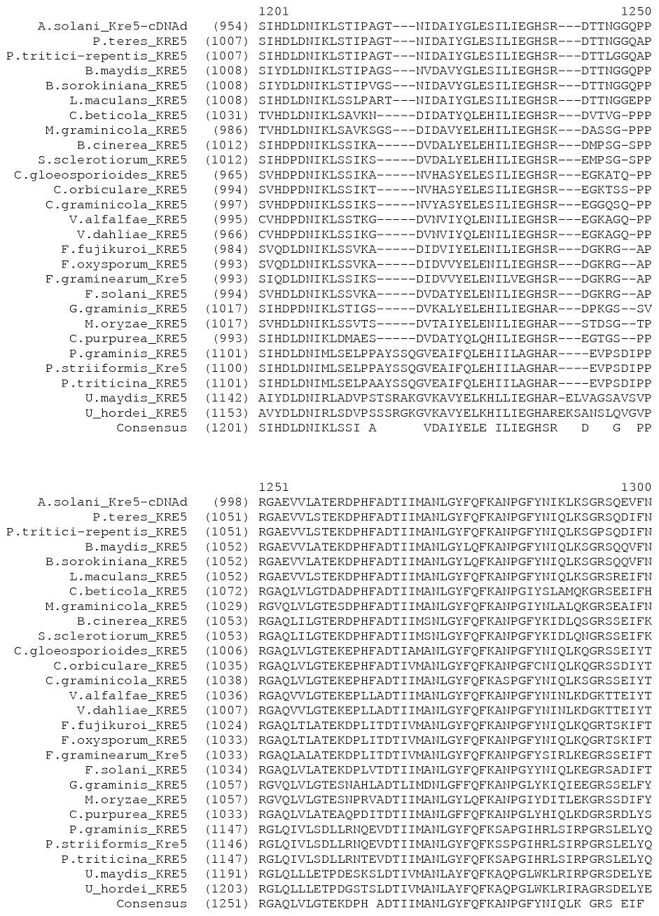

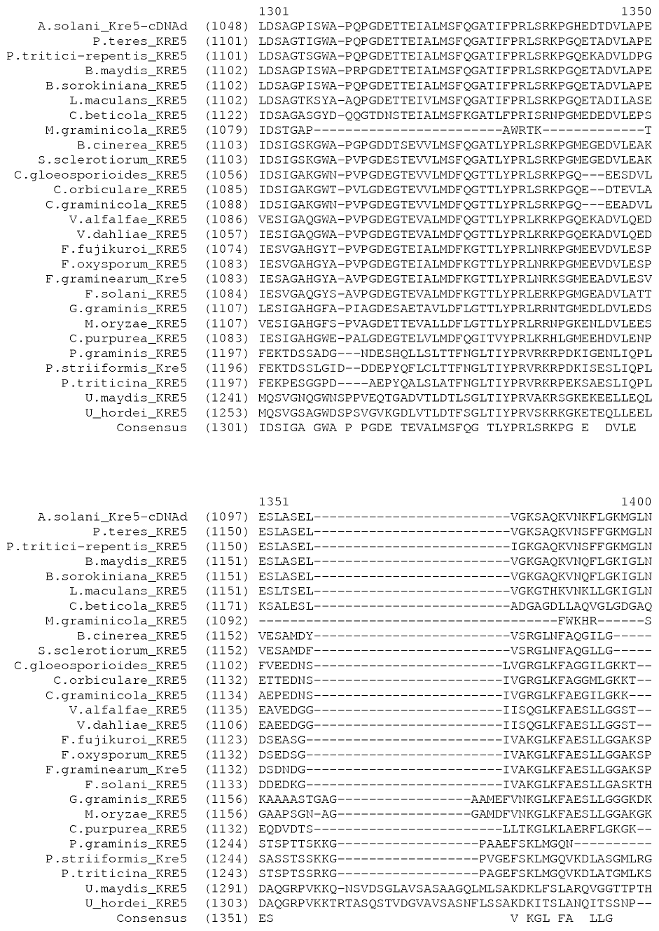

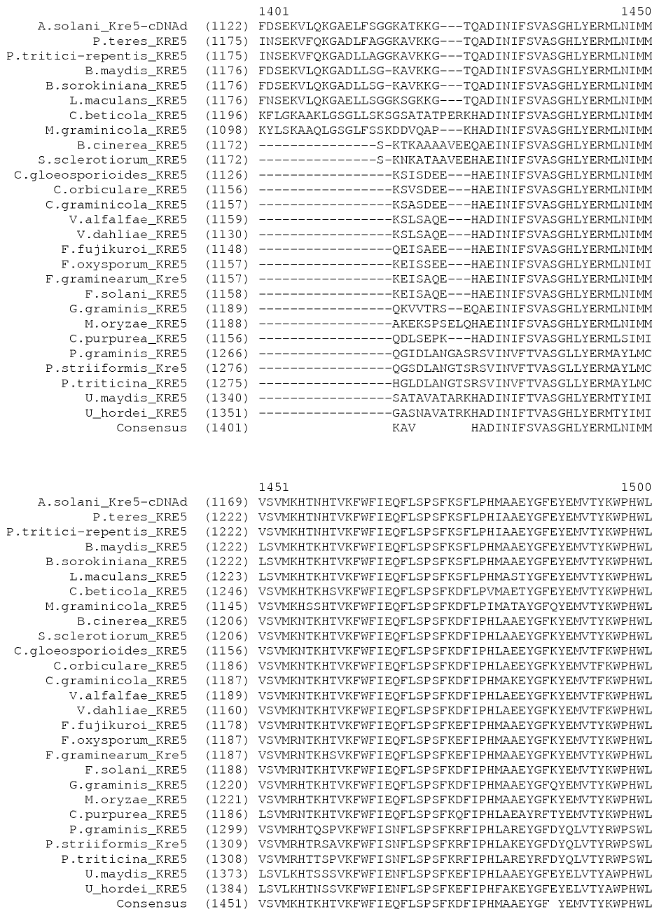

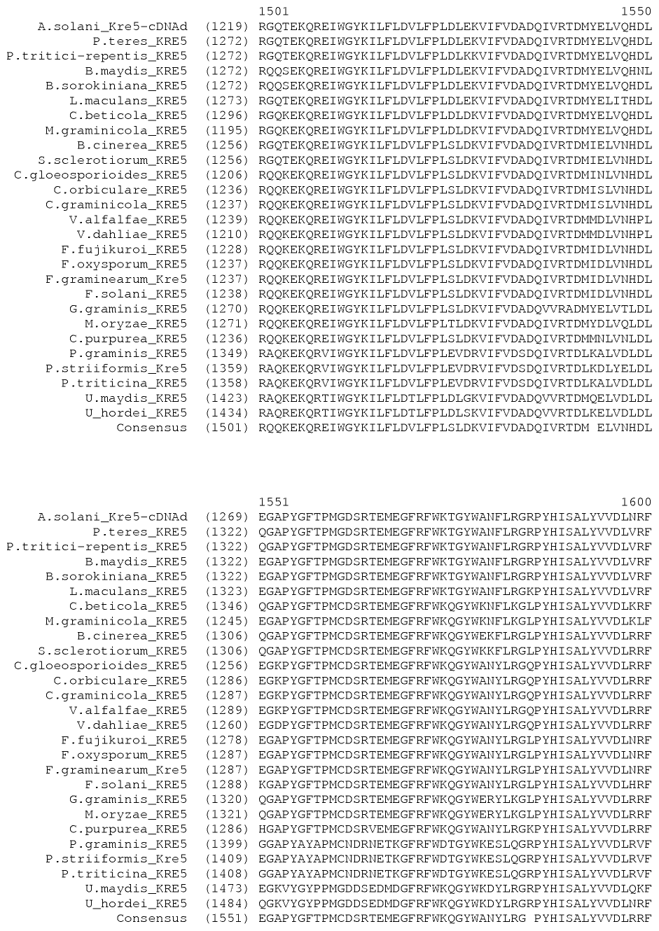

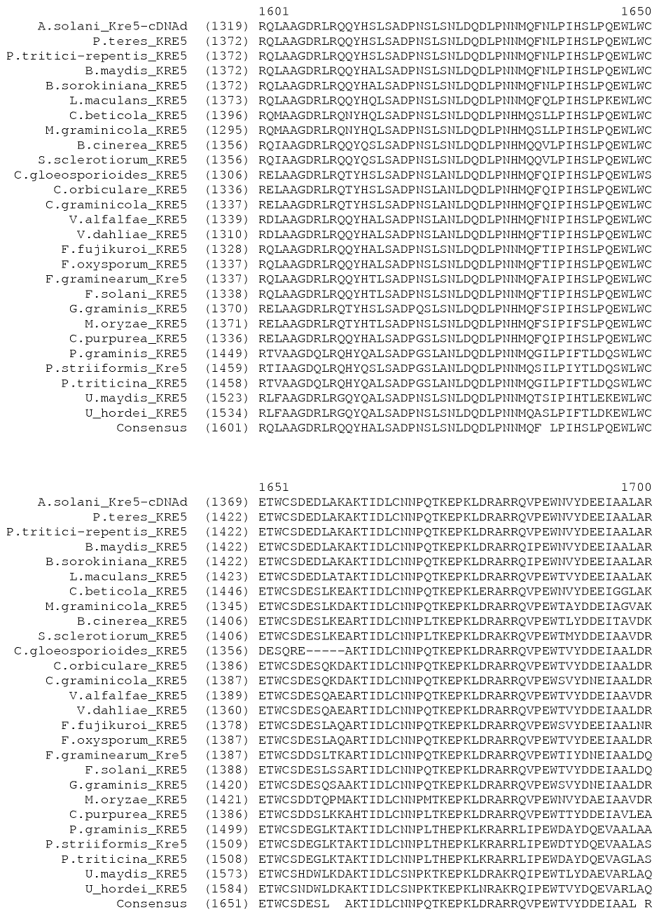

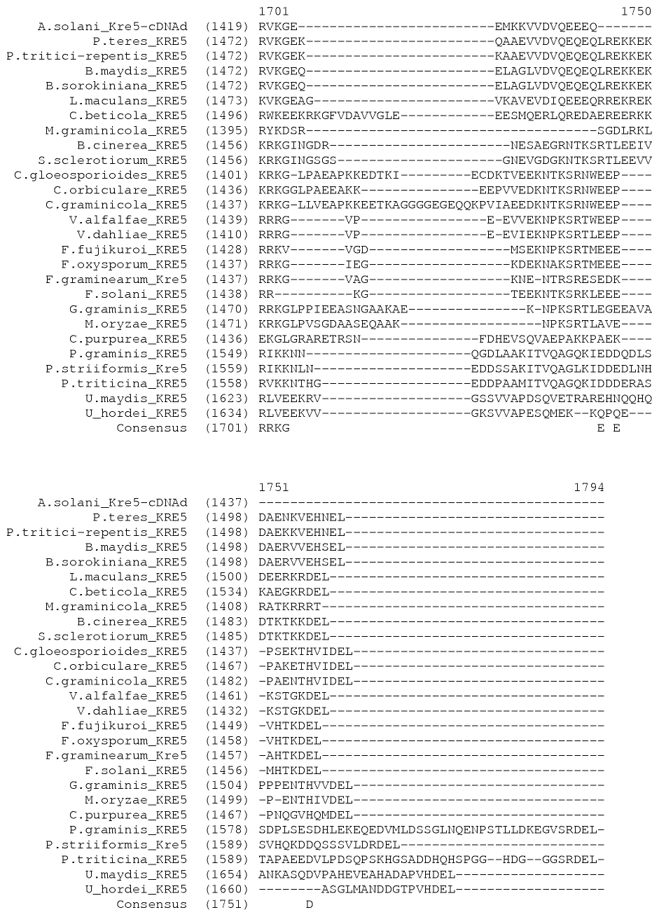

KRE5 proteins of filamentous fungi and yeasts have conserved protein domains, as shown in FIG. 2, which are suitable for the identification of a Kre5 gene of a fungal pathogen of interest. The KRE5 proteins have a size of 1290-1700 amino acids and an N-terminal secretion signal, a UDP-glucose:glycoprotein glucosyltransferase domain, a glucosyl-transferase family 8-like domain and an ER signal at the C-terminus. The following shows the KRE5 ascomycetes and basidiomycetes consensus sequence 1 comprising all strongly conserved amino acids in the aligned sequences, as shown in FIG. 4. These amino acids can be found from position 1431 to position 1693. X means any naturally occurring amino acid, Z means a gap or any naturally occurring amino acid:

(SEQ ID NO: 233)
INXFXVASGXLYERMXXXMXXSVXXXXXXXVKFWFIXXFL-
SPSFKXFXPHXXAXYXFXYXXVTYXWPXWLRXQXEKQRXIWGYKILFLDX
LF-
PLXXXXVIFVDXDQXVRXDXXXLXXXXLXGXXYXXXPMXXXXXXXXG-
FRFWXXGYWXXXLXGXPYHISALYVVDLXXFRXXAAGDXLRXXYXXLSAD
PXSLXNLDQDLPNXMQXXXPIXXLXXXWLWXXXXXXXZZZZZAXTIDLCX
NPXTXEPKLXRAXRXX-
PEWXXYDXE

The following shows the KRE5 ascomycetes and basidiomycetes consensus sequence 2 comprising all moderately and strongly conserved amino acids in the aligned sequences, as shown in FIG. 4, i.e. besides the strongly conserved amino acids, as given above in consensus sequence 1, also less conserved amino acids at other positions. These amino acids can be found from position 1431 to position 1693. X means any naturally occurring amino acid, Z means a gap or any naturally occurring amino acid:

FIG. 24. Map of the HIGS vector pGGubi_RGA2intronII_HIGS_GLRG-05611 which was used for the construction of the plant transformation vector p7U-ubi_RGA2intronII_HIGS_GLRG_05611.

FIG. 25. Map of the reporter gene vector pABM_ubiluci_GLRG_05611 which was used for the determination of transcriptional silencing activity of transgenic plants.

FIG. 26. Top: Map of the wheat transformation vector p6U-35S-MgKRE5-35. A 401 bp large fragment of the KRE5 gene of M. graminicola was inserted between the inverse transcribed 35S promoters of the construct. 35S=35S promoter, dsRNA MgUDP-G=sequence of the M. graminicola gene KRE5, hpt=hygromycin B resistance gene, Ubi-int=maize ubiquitin promoter, T35S=terminator of the 35S Cauliflower mosaic virus. Below: Enhanced Septoria blotch resistance of the wheat HIGS_{KRE5} line WA-601-T-035. The area under disease progression curve (AUDPC) from the disease scores taken 21, 26, 31 and 35 days after inoculation is shown for the transformation genotype Taifun, the non-transgenic segregants of the HIGS lines, the HIGS_{KRE5} line WA-601-T-035 and the reference lines Aurum and Taifun.

### EXAMPLES

Identification of KRE5 Proteins

KRE5 proteins of filamentous fungi and yeasts have conserved protein domains, as shown in FIG. 2, which are suitable for the identification of a Kre5 gene of a fungal pathogen of interest. The KRE5 proteins have a size of 1290-1700 amino acids and an N-terminal secretion signal, a UDP-glucose:glycoprotein glucosyltransferase domain, a glucosyl-transferase family 8-like domain and an ER signal at the C-terminus. The following shows the KRE5 ascomycetes and basidiomycetes consensus sequence 1 comprising all strongly conserved amino acids in the aligned sequences, as shown in FIG. 4. These amino acids can be found from position 1431 to position 1693. X means any naturally occurring amino acid, Z means a gap or any naturally occurring amino acid:

(SEQ ID NO: 233)
INXFXVASGXLYERMXXXMXXSVXXXXXXXVKFWFIXXFL-
SPSFKXFXPHXXAXYXFXYXXVTYXWPXWLRXQXEKQRXIWGYKILFLDX
LF-
PLXXXXVIFVDXDQXVRXDXXXLXXXXLXGXXYXXXPMXXXXXXXXG-
FRFWXXGYWXXXLXGXPYHISALYVVDLXXFRXXAAGDXLRXXYXXLSAD
PXSLXNLDQDLPNXMQXXXPIXXLXXXWLWXXXXXXXZZZZZAXTIDLCX
NPXTXEPKLXRAXRXXPEWXXYDXE

The following shows the KRE5 ascomycetes and basidiomycetes consensus sequence 2 comprising all moderately and strongly conserved amino acids in the aligned sequences, as shown in FIG. 4, i.e. besides the strongly conserved amino acids, as given above in consensus sequence 1, also less conserved amino acids at other positions. These amino acids can be found from position 1431 to position 1693. X means any naturally occurring amino acid, Z means a gap or any naturally occurring amino acid:

(SEQ ID NO: 234)
INIFSVASGHLYERMLNIMMVSVMKHTKHTVKFWFIEQFLSPSFKDFIPH
MAAEYGFXYE-
MVTYKWPHWLRQQKEKQREIWGYKILFLDVLFPLSLDKVIFVDADQIVRT
DMXELVNHD-
LEGAPYGFTPMCDSRTEMEGFRFWKQGYWANYL-
RGXPYHISALYVVDLRRFRQLAAGDRLRQQYHALSADPNSLSNLDQDLPN
N-
MQFXLPIHSLPQEWLWCETWCSDESLZZAKTIDLCNNPQT-
KEPKLDRARRQVPEWTVYDDE

Identification of KRE6 Proteins

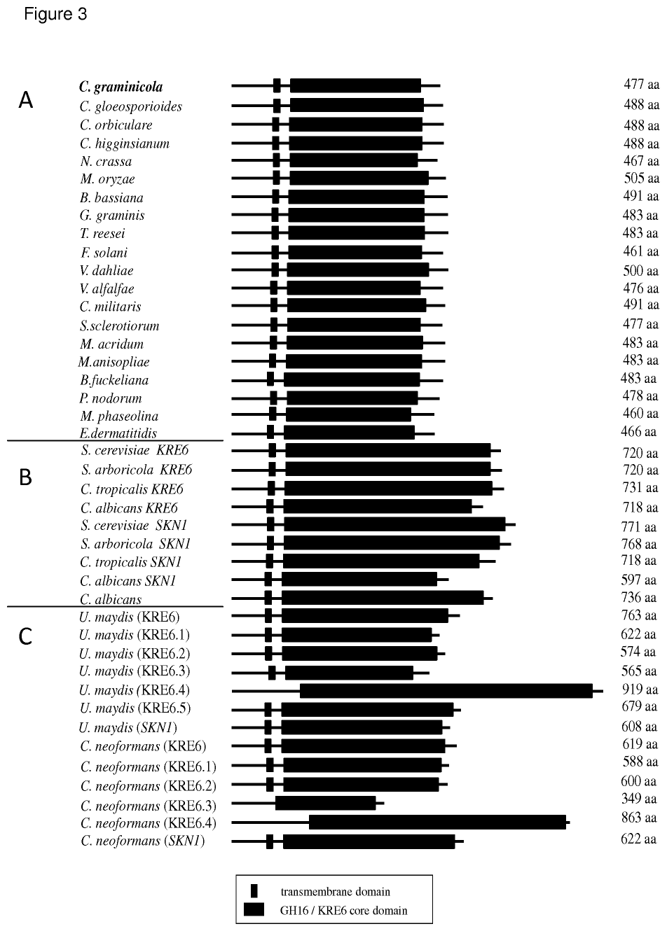

KRE6 proteins of filamentous fungi and yeasts have conserved protein domains, as shown in FIG. 3, which are suitable for the identification of a Kre6 gene of a fungal pathogen of interest. The KRE6 proteins have a size of 460-919 amino acids and have a conserved glycosyl hydrolases family 16 domain (Pfam domain PF00722.16).

KRE6 proteins of ascomycetes could be identified by the “KRE6 ascomycetes consensus sequence a

KRE6 Ascomycetes

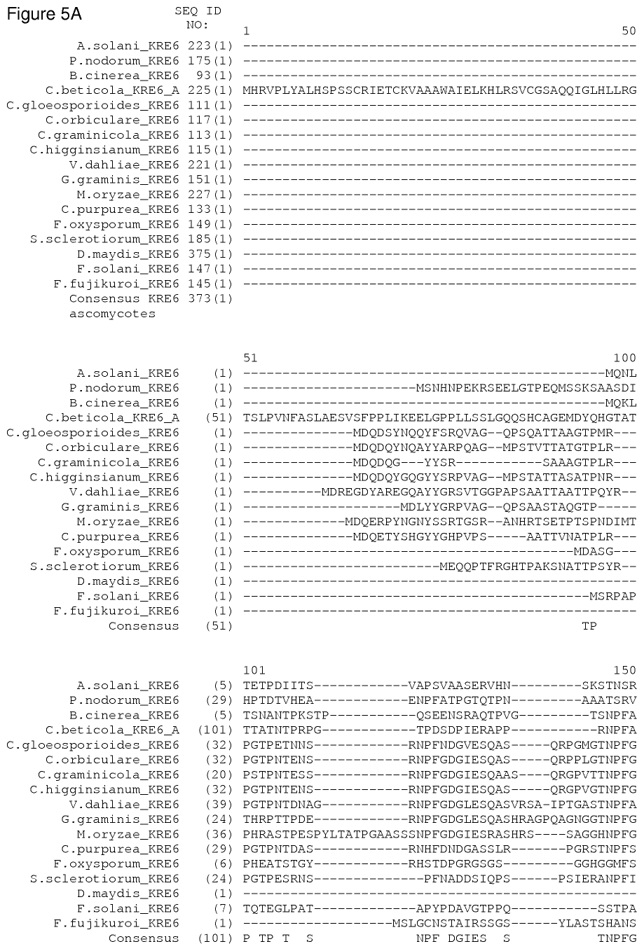

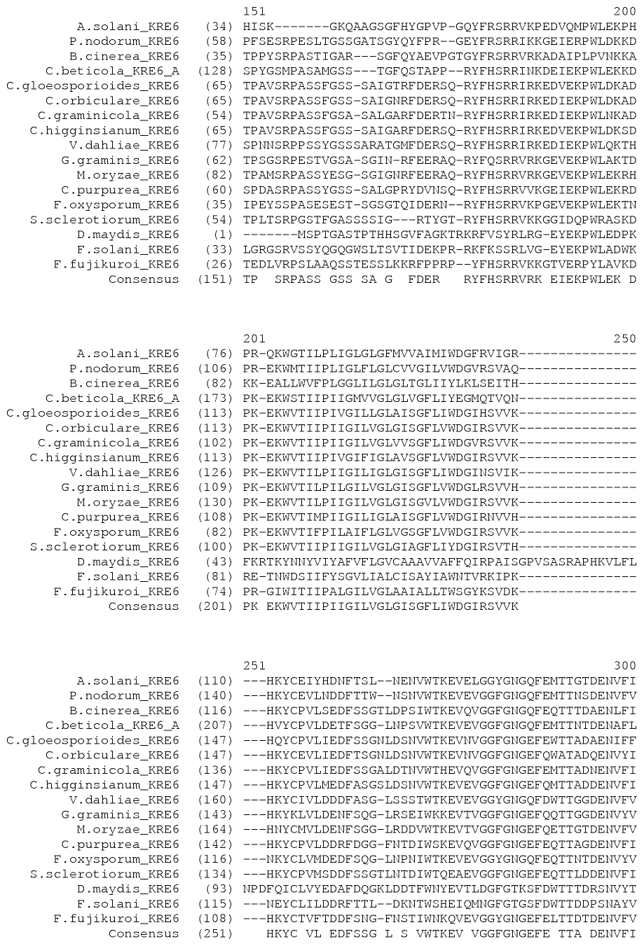

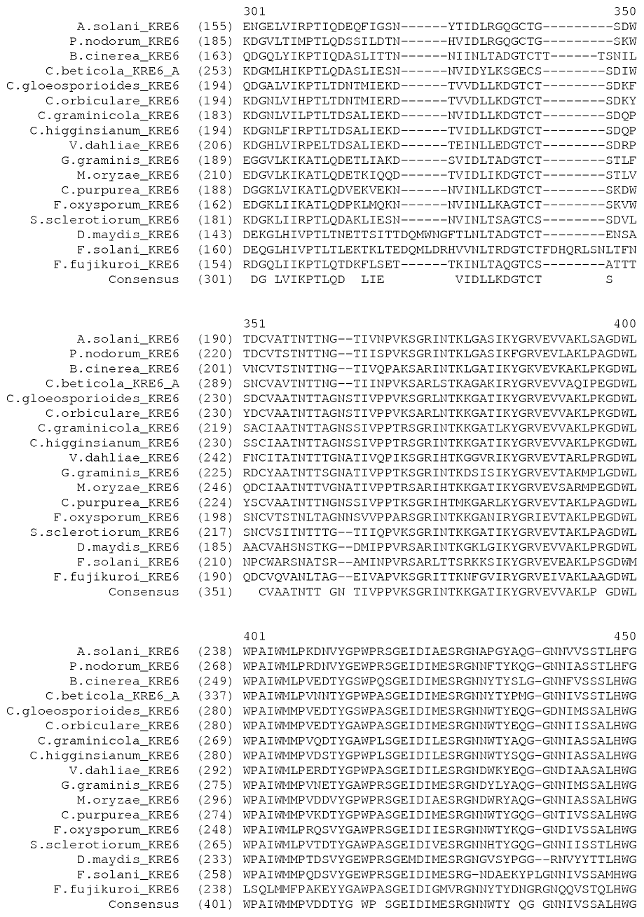

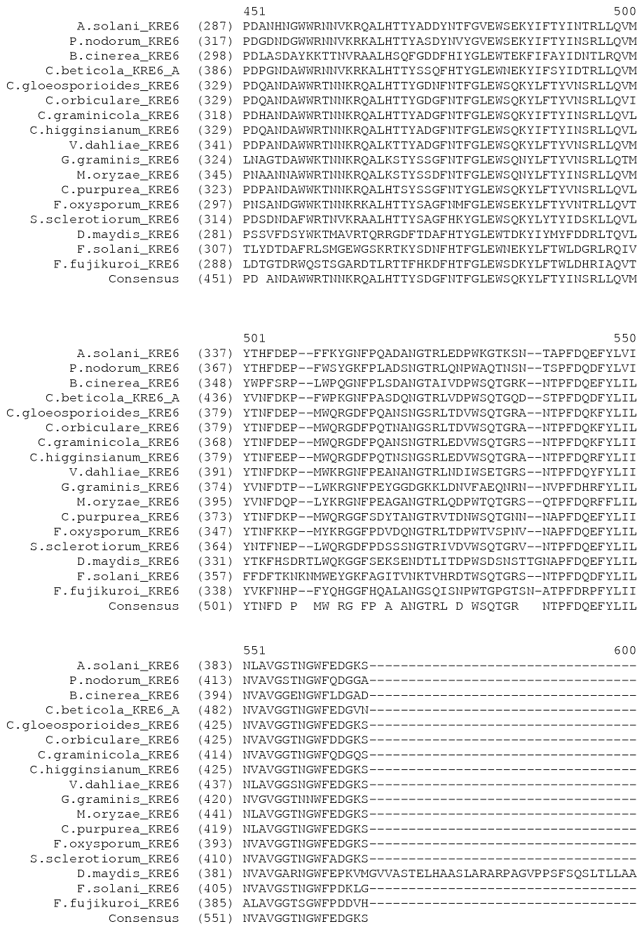

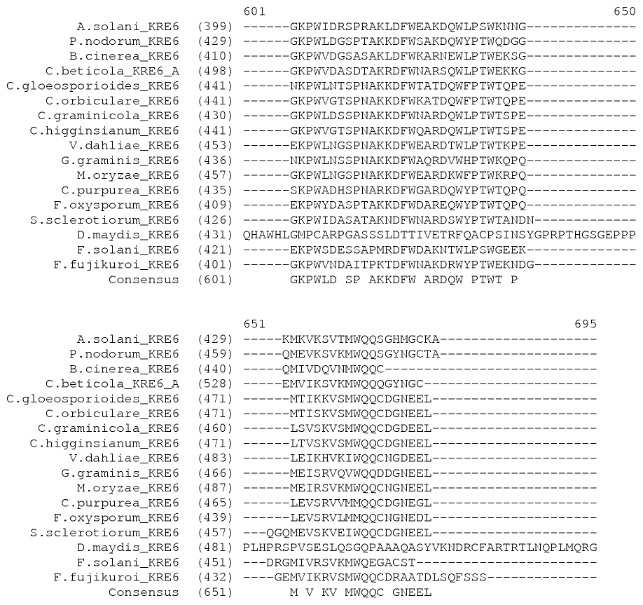

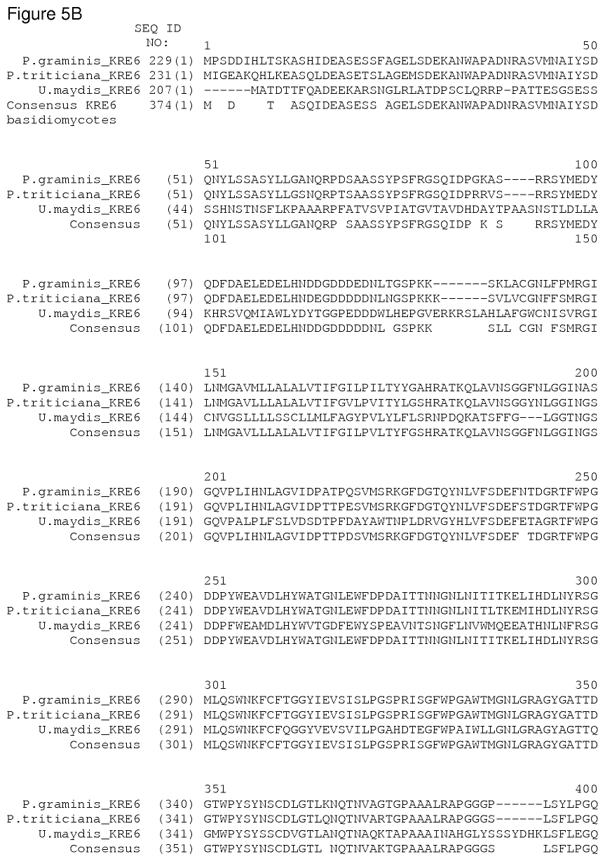

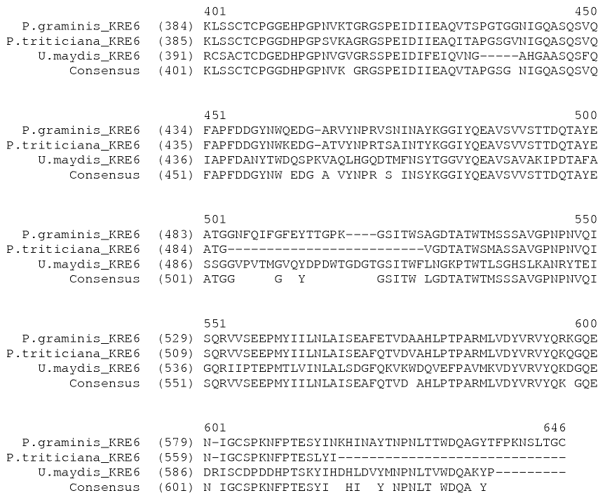

The following shows the KRE6 ascomycetes consensus sequence al comprising all strongly conserved amino acids in the aligned sequences, as shown in FIG. 5. These amino acids can be found from position 397 to position 450. X means any naturally occurring amino acid:

(SEQ ID NO: 235)
GDWXXXXXXMXPXXXXYGXWPXSGEXDIXXXRGXXXXXXXXXXXXXXXXX
XHXG

The following shows the KRE6 ascomycetes consensus sequence a2 comprising all moderately and strongly conserved amino acids in the aligned sequences, as shown in FIG. 5, i.e. besides the strongly conserved amino acids, as given above in consensus sequence a1, also less conserved amino acids at other positions. These amino acids can be found from position 397 to position 450. X means any naturally occurring amino acid:

(SEQ ID NO: 236)
GDWLWPAIWMMPVDDTYGXWPXSGEIDIMESRGNNWTYXQGXGNNIVSSA
LHWG

The following shows the KRE6 ascomycetes consensus sequence b1 comprising all strongly conserved amino acids in the aligned sequences, as shown in FIG. 5. These amino acids can be found from position 514 to position 561. X means any naturally occurring amino acid; Z means a gap or any naturally occurring amino acid:

(SEQ ID NO: 237)
GXFXXXXXXXXXXXXXXXXXXXZZXXXPFDXXFXLXXXXXVGXXXXWF

The following shows the KRE6 ascomycetes consensus sequence b2 comprising all moderately and strongly conserved amino acids in the aligned sequences, as shown in FIG. 5, i.e. besides the strongly conserved amino acids, as given above in consensus sequence b1, also less conserved amino acids at other positions. These amino acids can be found from position 514 to position 561. X means any naturally occurring amino acid; Z means a gap or any naturally occurring amino acid:

(SEQ ID NO: 238)
GXFPXAXANGTRLXDXWSQTGRXZZNTPFDQEFYLILNVAVGGTNGWF

KRE6 Basidiomycetes

The following shows the KRE6 basidiomycetes consensus sequence al comprising all strongly conserved amino acids in the aligned sequences, as shown in FIG. 5. These amino acids can be found from position 201 to position 435. X means any naturally occurring amino acid. Z means a gap or any naturally occurring amino acid:

(SEQ ID NO: 239)
GQVPXXXXXXXVXXXXXXXXXXXXXXXDXXXYNLVFSDEFXTXGRTFWPG
DDPXWE-
AXDLHYWXTGXXEWXXPXAXXTXNGXLNXXXXXEXXHXLNXRSGMLQSWN
KFCFXG-
GYXEVSXXLPGXXXXXGFWPXXWXXGNLGRAGYXXTTXGXWPY-
SYXSCDXGTLXNQTNXXXTXPAAAXXAXGXXXZZZZZZLSXLXGQXX-
SACTCXGXXHPGPXVXXGRXSPEIDIXEXQXXX

The following shows the KRE6 basidiomycetes consensus sequence a2 comprising all moderately and strongly conserved amino acids in the aligned sequences, as shown in FIG. 5, i.e. besides the strongly conserved amino acids, as given above in consensus sequence a1, also less conserved amino acids at other positions. These amino acids can be found from position 201 to position 435. X means any naturally occurring amino acid. Z means a gap or any naturally occurring amino acid:

(SEQ ID NO: 240)
GQVPLIHNLAGVIDPTTPDSVMSRKGFDGTQYNLVFSDEFXTDGRTFWPG
DDPYWEAV-
DLHYWATGNLEWFDPDAITTNNGNLNITITKELIHDLNYRSGMLQSWNKF
CFT-
GGYIEVSISLPGSPRISGFWPGAWTMGNLGRAGYGATTDGTWPY-
SYNSCDLGTLXNQTNVAKTGPAAALRAPGGGSZZZZZZLSFLPGQKLSSC
TCPGG-
DHPGPNVKXGRGSPEIDIIEAQVTA

The following shows the KRE6 basidiomycetes consensus sequence b1 comprising all strongly conserved amino acids in the aligned sequences, as shown in FIG. 5. These amino acids can be found from position 443 to position 612. X means any naturally occurring amino acid. Z means a gap or any naturally occurring amino acid:

(SEQ ID NO: 241)
GXASQSXQXAP-
FDXXYXWXXXXZXXXXXXXXXXXNXYXGGXYQEAVSXXXXXXXTAXXXXG
ZZZZZZZZZZZZZZZZZZZZZZZZZXXXXXTWXXXXXXXXXNXXXXIXQR
XXXXEP-
MXXXXNLAXSXXFXXVXXXXXXXPAXMXVDYVRVYQXXGQEXZIXCXPXX
XPT

The following shows the KRE6 basidiomycetes consensus sequence b2 comprising all moderately and strongly conserved amino acids in the aligned sequences, as shown in FIG. 5, i.e. besides the strongly conserved amino acids, as given above in consensus sequence b1, also less conserved amino acids at other positions. These amino acids can be found from position 443 to position 612. X means any naturally occurring amino acid. Z means a gap or any naturally occurring amino acid:

(SEQ ID NO: 242)
GQASQSVQFAPFDDGYNWXEDGZAXVYNPRXSXINSYKG-
GIYQEAVSVVSTTDQTAYEATGGZZZZZGZZYZZZZZZZZZGSITWZLGD
TATWTMSSSA-
VGPNPNVQISQRVVSEEPMYIILNLAISEAFQTVDXAHLPTPARMLVDYV
RVYQKXGQEN-
ZIGCSPKNFPT

KRE5 and KRE6 of C. graminicola are Functional Homologs of the Corresponding K1 Killer Toxin Resistance Genes of Yeast

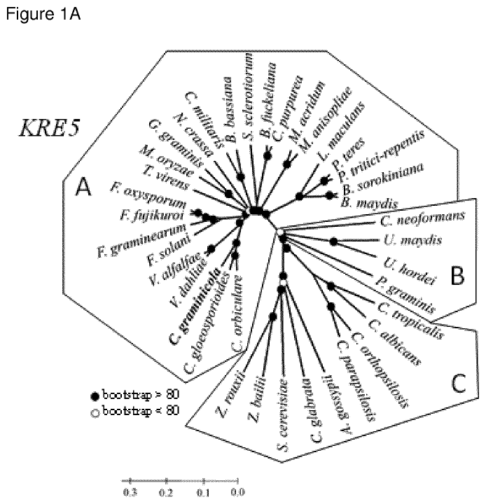

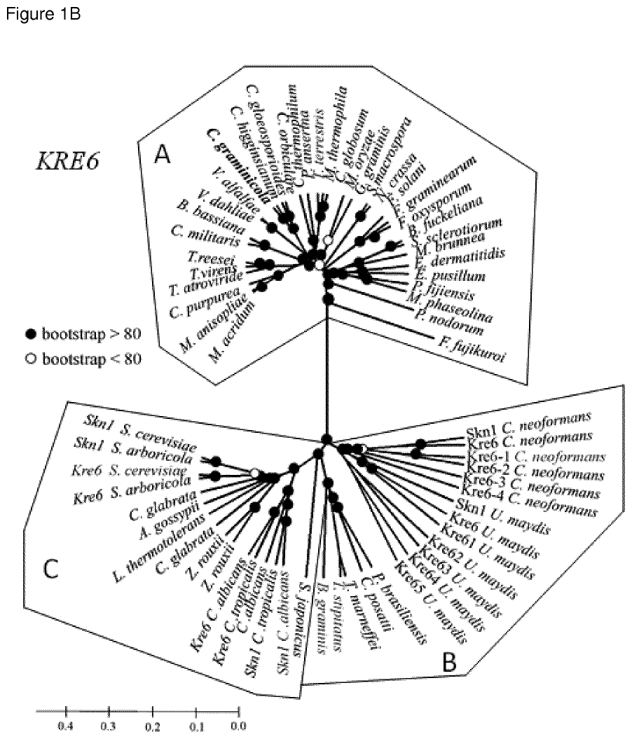

BLASTX searches performed with Kre5 and Kre6 proteins of the yeast S. cerevisiae, the filamentous ascomycetes Magnaporthe oryzae, Neurospora crassa, Colletortichun gloeosporioides, Fusarium solani and the dimorphic basidiomycete Ustilago maydis suggested that single copy KRE5 and KRE6 genes exist in the annotated genome of C. graminicola (http://www.broadinstitute.org/annotation/genome/colletotrichum_group/FeatureSearch.html; O'Connell et al., 2012, Nat. Genet. 44: 962-970). The phylogenetic tree calculated on the basis of the derived amino acid sequences shows that the Kre5 and Kre6 proteins of filamentous ascomycetes form clades, distinct from the Kre proteins of yeasts and basidiomcetes (FIG. 1). On the amino acid level, Kre5 and Kre6 of C. graminicola share 69% and 60% sequence identity with the corresponding proteins of M. oryzae, and 65% and 63% with those of Fusarium solani, respectively. However, the C. graminicola Kre5 and Kre6 proteins are only 23%, and 30% identical to the proteins of S. cerevisiae. Plants also synthesize putative Kre5-like glycosyltransferase proteins. The proteins of Arabidopsis (NP_177278.3) and maize (AFW73943) share only 32% and 54% identity with Kre5 of C. graminicola. Kre6 homologs do not occur in plants.

The size of the predicted fungal Kre5 proteins ranges from 1293 (Ashbya gossypii) to 1678 amino acids (Ustilago maydis), with Kre5 of C. graminicola containing 1492 amino acids. All Kre5 proteins contain an N-terminal secretion signal and two different conserved glycosyl-transferase domains. Importantly, all Kre5 proteins show a C-terminal ER retention signal. While Kre5 proteins of the Colletotrichum species shown here and of Neurospora crassa exhibit an IDEL retention signal, Kre5 proteins of the majority of filamentous ascomycetes have a KDEL-, and the majority of those of dimorphic fungi have a HDEL tetrapeptide (FIG. 2).

The size of the predicted fungal Kre6 proteins ranges from 349 (Kre6.3 of C. neoformans) to 919 amino acids (Kre6.4 of Ustilago maydis), with Kre6 of C. graminicola consisting of 477 amino acids. The vast majority of Kre6 proteins contains a single N-terminal transmembrane domain and a prominent central family 16 glycohydrolase core domain (FIG. 3). In contrast to Kre5 proteins, an apparent ER retention signal does not exist in Kre6 proteins.

The function of KRE5 and KRE6 of C. graminicola was confirmed by complementation of yeast KRE5 and KRE6 deletion strains Y21633 and Y05574. The S. cerevisiae Δkre5 and Δkre6 mutants are viable but exhibit severe growth defects on osmotically non-stabilized medium (FIG. 6). These growth defects were fully rescued by osmotically stabilizing the YPD medium with 1 M sorbitol (FIG. 6, YPDS). Both Δkre5 and Δkre6 strains were hypersensitive to the chitin synthesis inhibitor Calcofluor White and showed increased resistance to the viral killer toxin K1, which needs to bind to β-1,6-glucan in order to execute its toxic effect.

Approx. one million yeast transformants expressing the C. graminicola KRE5 and KRE6 cDNAs under control of the constitutive yeast alcohol dehydrogenase 1 promoter (PADH1) were generated, and four independent transformants each (TKRE51-4 and TKRE61-4) were randomly chosen and used in growth assays. All yeast transformants expressing C. graminicola KRE5 and KRE6 cDNA showed growth comparable to that of the WT strain, irrespective of osmotic support, and resistance to Calcofluor White. As expected, Δkre5 and Δkre6 strains expressing C. graminicola KRE5 and KRE6 cDNAs were susceptible to the killer toxin K1 (FIG. 6A). Cells of Δkre5 and Δkre6 strains were significantly larger than those of the WT strain, likely due to severe cell wall defects in the mutant strains (FIG. 6B). Interestingly, some of the Δkre6 cells formed irregularly shaped filaments (FIG. 6B, Δkre6, insert). Yeast transformants expressing C. graminicola KRE5 and KRE6 cDNA exhibited size and shape comparable to the WT, indicating that the KRE5 and KRE6 cDNAs were fully functional. The empty expression vector pAG300 (TpAG300) did not rescue the phenotype of Δkre5 and Δkre6 strains (FIGS. 6A and B).

Collectively, sequence similarities with functionally characterized proteins, predictions of programs such as SignalP Server v. 7.0 and TMHMM Server v. 2.0, as well as the yeast complementation experiments strongly suggest that Kre5 and Kre6 of C. graminicola represent functional secreted UDP-glucose-dependent glycosyltransferase- and membrane-integral glycohydrolase-like proteins, respectively.

Sequence data for KRE5 and KRE6 genes of various fungi are available from the NCBI (National Centre for Biotechnology Information; National Library of Medicine 38A, Bethesda, Md. 20894, USA) under the following accession numbers:

KRE5 Genes and Proteins

The following list indicates the organism and the accession numbers under which amino acid and nucleotide sequences of KRE5 are to be found. These amino acid and nucleotide sequences are disclosed, in the order of appearance, in the sequence listing under SEQ ID NOs: 1 to 76, whereby uneven numbers refer to the KRE5 protein and the following even numbers refer to the coding sequence encoding the preceding protein.

A. gossypii, Ashbya gossypii (NP_984460.1); B. bassiana, Beauveria bassiana (EJP60779.1); B. maydis, Bipolaris maydis (EN100093.1); B. sorokiniana, Bipolaris sorokiniana (EMD66340.1); B. fuckeliana, Botryotinia fuckeliana (synonym of B. cinerea, Botrytis cinerea) (XP_001552779.1); C. graminicola, Colletotrichum graminicola (EFQ30467.1); C. gloeosporioides, Colletotrichum gloeosporioides, (ELA26384.1); C. orbiculare, Colletotrichum orbiculare (ENH83729.1); C. albicans, Candida albicans (XP_720119.1); C. glabrata, Candida glabrata (XP_445924.1); C. orthopsilosis, Candida orthopsilosis (XP_003867612.1); C. parapsilosis, Candida parapsilosis (CCE40308.1); C. tropicalis, C. tropicalis, Candida tropicalis (XP_002548275.1); C. purpurea, Claviceps purpurea (CCE27564.1); C. militaris, Cordyceps militaris (EGX96522.1); C. neoformans, Cryptococcus neoformans (XP_568822.1); F. fujikuroi, Fusarium fujikuroi (CCT67477.1); F. graminearum, Fusarium graminearum (XP_388116.1); F. oxysporum, Fusarium oxysporum (EGU85390.1); F. solani, Fusarium solani (XP_003040714.1); G. graminis, Gaeumannomyces graminis (EJT75191.1); L. maculans, Leptosphaeria maculans (XP_003837705.1); M. oryzae, Magnaporthe oryzae (XP_003716409.1); M. acridum, Metarhizium acridum (EFY92405.1); M. anisopliae, Metarhizium anisopliae (EFY99515.1); N. crassa, Neurospora crassa (XP_959471.1); P. teres, Pyrenophora teres (XP_003302012.1); P. tritici-repentis, Pyrenophora tritici-repentis (XP_001932611.1); P. graminis, Puccinia graminis (XP_003335101.2); T. virens, Trichoderma virens (EHK19050.1); S. sclerotiorum, Sclerotinia sclerotiorum (XP_001587888.1); S. cerevisiae, Saccharomyces cerevisiae (EEU07540.1); U. maydis, Ustilago maydis, (XP_757659.1); U. hordei, Ustilago hordei (CCF53656.1); V. alfalfae, Verticillium alfalfae (XP_003001998.1); V. dahliae, Verticillium dahliae (EGY20293.1); Z. bailii, Zygosaccharomyces bailii (CDF90600.1); Z. rouxii, Zygosaccharomyces rouxii (XP_002496624.1).

The following list indicates the organisms comprising the KRE5 proteins and genes which are disclosed, in the order of appearance, under SEQ ID Nos: 77 to 88.

Alternaria solani, Cercospora beticola, Mycosphaerella graminicola, Puccinia striiformis, Puccinia triticina, and Verticillium dahliae.

Especially preferred, the inhibitory nucleic acid molecule capable of inhibiting the expression of (a) KRE5 and/or KRE6 gene(s) in a fungus inhibits the expression of the KRE5 gene of the following species: Alternaria solani, Ashbya gossypii, Bipolaris maydis, Bipolaris sorokiniana, Botryotinia fuckeliana, Cercospora beticola, Colletotrichum graminicola, Colletotrichum gloeosporioides, Colletotrichum orbiculare, Claviceps purpurea, Fusarium fujikuroi, Fusarium graminearum, Fusarium oxysporum; Fusarium solani, Gaeumannomyces graminis, Leptosphaeria maculans, Magnaporthe oryzae, Mycosphaerella graminicola, Pyrenophora teres, Pyrenophora tritici-repentis, Puccinia graminis, Puccinia striiformis, Puccinia triticina, Sclerotinia sclerotiorum, Ustilago maydis, Ustilago hordei, Verticillium alfalfa, and Verticillium dahliae (the accession numbers are, as indicated above).

KRE6 Genes and Proteins

The following list indicates the organism and the accession numbers under which amino acid and nucleotide sequences of KRE6 are to be found. These amino acid and nucleotide sequences are disclosed, in the order of appearance, in the sequence listing under SEQ ID NOs: 89 to 222, whereby uneven numbers refer to the KRE6 protein and the following even numbers refer to the coding sequence encoding the preceding protein.

B. bassiana, Beauveria bassiana (EJP65479.1); B. graminis, Blumeria graminis (CCU77212.1); B. fuckeliana, Botryotinia fuckeliana (synonym of B. cinerea, Botrytis cinerea) (XP_001549048.1); C. albicans, Candida albicans (KRE6) (EEQ44379.1); C. albicans, Candida albicans (KRE6) (EEQ44618.1); C. albicans, Candida albicans (SKN1) (P87024.1); C. glabrata, Candida glabrata (XP_446183.1); C. glabrata, Candida glabrata (XP_447683.1); C. tropicalis, Candida tropicalis (SKN1) (XP_002547983.1); C. tropicalis, Candida tropicalis (KRE6) (XP_002547982.1); C. globosum, Chaetomium globosum (EGY21198.1); C. gloeosporioides, Colletotrichum gloeosporioides (ELA25364.1); C. graminicola, Colletotrichum graminicola (EFQ32556.1); C. higginsianum, Colletotrichum higginsianum (CCF34135.1); C. orbiculare, Colletotrichum orbiculare (ENH85681.1); C. militaris, (EGX96292.1); C. neoformans, Cryptococcus neoformans (SKN1) (CNAG_00897.2); C. neoformans, Cryptococcus neoformans (KRE6) (CNAG_00914.2); C. neoformans, Cryptococcus neoformans (KRE61) (CNAG_06835.2); C. neoformans, Cryptococcus neoformans (KRE62) (CNAG_06832.2); C. neoformans, Cryptococcus neoformans (KRE63) (CNAG_06031.2); C. neoformans, Cryptococcus neoformans (KRE64) (CNAG_05815.2); C. purpurea, Claviceps purpurea (CCE32615.1); C. posadasii, Coccidioides posadasii (EFW17515.1); C. thermophilum, Chaetomium thermophilum (EGS22614.1); E. pusillum, Endocarpon pusillum (ERF71048.1); E. dermatitidis, Exophiala dermatitidis (EHY52267.1); F.graminearum, Fusarium graminearum (XP_388710.1); F. fujikuroi, Fusarium fujikuroi (CCT73009.1); F. solani, Fusarium solani (XP_003041595.1); F. oxysporum, Fusarium oxysporum (ENH65541.1); G. graminis, Gaeumannomyces graminis (EJT70769.1); L. thermotolerans, Lachancea thermotoleran (XP_002555132.1); M. oryzae, (XP_003721228.1); M. brunnea, Marssonina brunnea (EKD14562.1); M. thermophila, Myceliophthora thermophila (XP_003666443.1); M. acridum, Metarhizium acridum (EFY93704.1); M. anisopliae, Metarhizium anisopliae (EFY95465.1); M. phaseolina, Macrophomina phaseolina (EKG13666.1); N. crassa, (XP_958019.1); P. anserina, Podospora anserina (XP_003437515.1); P. brasiliensis (EEH42350.1); P. tritici-repentis, Pyrenophora tritici-repentis (KRE6) (XP_001935327.1); P. nodorum, Phaeosphaeria nodorum (XP_001797916.1); S. cerevisae, Saccharomyces cerevisiae (Kre6) (NP_015485.1); S. arboricola, Saccharomyces arboricola (Kre6) (EJS41292.1); S. cerevisae, Saccharomyces cerevisiae (SKN1) (EGA86662.1); S. arboricola, Saccharomyces arboricola (SKN1) (EJS43532.1); S. sclerotiorum, Sclerotinia sclerotiorum (XP_001593690.1); S. macrospora, (XP_003344223.1); S. japonicus, (XP_002173652.1); Z. rouxii, Zygosaccharomyces rouxii (XP_002496208.1); Z. rouxii, Zygosaccharomyces rouxii (XP_002496207.1); T. marneffei, Talaromyces marneffei (XP_002148725.1); T. stipitatus, Talaromyces stipitatus (XP_002485501.1); T. terrestris, Thielavia terrestris (XP_003651215.1); T. virens, Trichoderma virens (EHK18881.1); T. reesei, Trichoderma reesei (EGR46871.1); T. atroviride, Trichoderma atroviride (EHK45772.1); U. maydis, Ustilago maydis (XP_759188.1); U. maydis, Ustilago maydis (XP_761958.1); U. maydis, Ustilago maydis (XP_761956.1); U. maydis, Ustilago maydis (XP_761865.1); U. maydis, Ustilago maydis (XP_757004.1); U. maydis, P. fijiensis, Pseudocercospora fijiensis (EME88366.1); V. alfalfae, Verticillium alfalfae (XP_003008063.1); V. dahliae, Verticillium dahliae (EGY21198.1).

The following list indicates the organisms comprising the KRE6 proteins and genes which are disclosed, in the order of appearance, under SEQ ID Nos: 223 to 232.

Alternaria solani, Cercospora beticola, Magnaporthe oryzae, Puccinia graminis, and Puccinia triticiana.

The KRE6 amino acid and nucleotide sequences of D. maydis, Diplodia maydis are disclosed in the sequence listing under SEQ ID NOs: 375 and 376, respectively.

Especially preferred, the inhibitory nucleic acid molecule capable of inhibiting the expression of (a) KRE5 and/or KRE6 gene(s) in a fungus inhibits the expression of the KRE6 gene of the following species: Alternaria solani, Blumeria graminis, Botryotinia fuckeliana, Cercospora beticola, Claviceps purpurea, Colletotrichum graminicola, Colletotrichum higginsianum, Colletotrichum orbiculare, Fusarium fujikuroi, Fusarium solani, Fusarium oxysporum, Gaeumannomyces graminis, Magnaporthe oryzae, Puccinia graminis, Puccinia triticiana, Pyrenophora tritici-repentis, Phaeosphaeria nodorum, Sclerotinia sclerotiorum, Ustilago maydis, Verticillium alfalfae, and Verticillium dahlia dahliae (the accession numbers are, as indicated above; for Ustilago maydis, the accession number is XP_759188.1)

Expression of KRE5 and KRE6 and Cell Wall β-1,6-Glucan Contents are Drastically Down-Regulated in Biotrophic Hyphae

To study localization of Kre5 and Kre6 as well as expression of KRE5 and KRE6 in infection structure, C. graminicola strains were generated carrying single copies of KRE5:mCherry or KRE6:mCherry, replacing the KRE5 and KRE6 WT genes (FIGS. 7A and B).

Gene replacements were performed in a strain, in which the WT GLS1 allele had been replaced by a GLS1:eGFP fusion (Oliveira-Garcia E and Deising H B, 2013). The four independent and randomly chosen transformants of each strain did not differ in colony phenotype, vegetative growth rates, sporulation rates and virulence on maize (FIG. 7C-F).

Fluorescence microscopy of vegetative hyphae revealed that both the KRE5:mCherry GLS1:eGFP and the KRE6:mCherry GLS1:eGFP replacement strains showed an assembly of strongly fluorescing vesicles primarily in the hyphal tip region (FIG. 8A). Kre5:mCherry fluorescence did not co-localize with fluorescing macro-vesicles harboring Gls1:eGFP, as indicated by the presence of distinct vesicles in different colors in the merged fluorescence micrographs (FIG. 8A, merge; Kre5:mCherry; Gls1:eGFP). In contrast, Kre6:mCherry and Gls1:eGFP co-localized in macro-vesicles, as only superposed fluorescing vesicles were visible in the merged micrographs (FIG. 8A, merge).

To investigate localization of Kre5 and/or Kre6 after fusion of the vesicles with the plasma membrane, protoplasts were generated, using fungal cell wall lyzing enzymes from Trichoderma harzianum, and subsequently incubated for up to 24 h (FIG. 8B). Six hours after protoplasting, vesicles fluoresced brightly, and co-localization of Kre6:mCherry and Gls1:eGFP, but not of Kre5:mCherry and Gls1:eGFP, was clearly evident (FIG. 8B, 6 h; compare with FIG. 8A, merge). Interestingly, 24 hours after protoplasting, the vesicles had fused with the plasma membrane, again resulting in co-localization of Kre6:mCherry and Gls1:eGFP in the membrane (FIG. 8B, 24 h), suggesting that these 13-glucan-forming enzymes may directly cooperate in cell wall branched β-glucan synthesis at the plasma membrane. However, in accordance with the secretion signal and the lack of membrane-spanning domains in Kre5, the enzyme is likely secreted and escaped from the protoplasts (FIG. 8B, 24 h).

KRE genes are thought to be constitutively expressed during vegetative growth. This, however, may not be the case in infection hyphae, as Oliveira-Garcia E and Deising, H B, 2013, have recently shown that expression of the β-1,3-glucan synthase gene GLS1 is rigorously down-regulated in biotrophic infection structures of C. graminicola, and that avoidance of exposure of β-1,3-glucan is required to evade β-glucan-triggered defense responses in maize. Correspondingly, as both β-1,3- and β-1,6-linkages are likely present in structural β-glucans triggering defense responses, infection structure-specific regulation of KRE5 and KRE6 genes, like of GLS1, may be required to establish a compatible interaction between this pathogen and its host. So far, to our knowledge, expression of KRE genes and synthesis of β-1,6-glucan has not been analyzed during any fungal infection process on plants. Unfortunately, as infection structure differentiation of C. graminicola on maize leaves does not occur in a synchronized fashion, neither RT-qPCR-based quantification of KRE5 and KRE6 transcript abundance nor chemical quantification of β-1,6-glucan in individual infection hyphae are feasible. We therefore used the KRE5:mCherry and KRE6:mCherry replacement strains of C. graminicola to quantify KRE5 and KRE6 expression in individual infection structures of C. graminicola by measuring mCherry fluorescence, and to compare KRE gene expression with the expression of GLS1, measured as GLS1:eGFP fluorescence (Oliveira-Garcia and Deising, 2013) (FIG. 10). Virulence of KRE5:mCherry and KRE6:mCherry replacement strains did not differ from that of the WT strain (FIG. 7F). Strong Kre5:mCherry and Kre6:mCherry fluorescence was observed in non-germinated conidia and in appressoria. Importantly, biotrophic infection vesicles and primary hyphae showed only background Kre5:mCherry and Kre6:mCherry fluorescence (FIGS. 10A and B, 24 HAI, ph; FIGS. 10C and D, ap; note that the fluorescence signal marked with an arrow in FIG. 3B was emitted by the germ tube and the conidium on the cuticle, not by the biotrophic hypha). In marked contrast to biotrophic hyphae, necrotrophic secondary hyphae formed by KRE5:mCherry and KRE6:mCherry strains exhibited strong fluorescence (FIGS. 10A and B, 72 HAI, sh; FIGS. 10C and D, sh). Like Kre5:mCherry and Kre6:mCherry fluorescence, Gls1:eGFP fluorescence was strong in conidia, appressoria and necrotrophic hyphae, but almost undetectable in biotrophic hyphae (FIGS. 10A and B, Gls1:eGFP). Thus, spatial distribution and intensities of mCherry and eGFP fluorescence intensities in KRE5:mCherry GLS1:eGFP and the KRE6:mCherry GLS1:eGFP replacement strains strongly suggests co-regulation of β-1,3-synthesis and β-1,6-bond formation.

As molecular probes for infection structure-specific detection of β-1,6-glucan are not commercially available, we generated a chimeric protein consisting of 231 amino acids of the β-1,6-glucan binding domain of endo-β-1,6-glucanase of C. graminicola (GLRG_00130.1) and 238 amino acids of the yellow fluorescent protein (YFP) of Aequoria victoria, yielding β-1,6-GBD:YFP (FIG. 11A). Expression of this construct in S. cerevisiae was driven by the alcohol dehydrogenase 1 promoter (PADH1), and the addition of a secretion signal and a His6-tag allowed simple affinity purification from yeast culture filtrates (FIG. 11B). Dot blot analyses revealed that this probe strongly decorated the β-1,6-glucan homopolymer pustulan, and, to minor extent, the β-1,3-glucan laminarin. Laminarin is as a β-1,3-glucan containing some β-1,6-linkages, and these β-1,6-linkages may explain labeling of commercially available laminarin. Other polymers tested, i.e. cellulose, mannan, chitin and chitosan only showed background or no labeling (FIGS. 110 and D). Therefore the β-1,6-GBD:YFP-probe was considered suitable for investigating β-1,6-glucan bonds of the infection structures of C. graminicola. In full agreement with the KRE5 and KRE6 gene expression data, the β-1,6-GBD:YFP probe decorated infection structures formed on the maize leaf cuticle, i.e. conidia, germ tubes and appressoria (FIG. 10E). As the β-1,6-GBD:YFP-probe did not diffuse into infected plant tissue, visualization of β-1,6-glucan was only possible in cross sections of infected leaves. Intriguingly, labeling experiments with cross-sectioned biotrophic and necrotrophic hyphae showed that β-1,6-glucan bonds are prominent in secondary hyphae, but almost undetectable in biotrophic infection vesicles and primary hyphae (FIG. 10E).

In conclusion, fluorescence microscopy revealed that KRE5 and KRE6 expression, and β-1,6-glucan contents of cell walls, are prominent in conidia, appressoria and necrotrophic hyphae but are dramatically down-regulated in biotrophic infection structures of C. graminicola.

RNAi-Mediated Reduction of KRE5 and KRE6 Transcript Abundance Causes Severe Cell Wall Defects, Impaired Invasive Growth, and Hyper-Pigmentation of Vegetative Hyphae

While deletion of KRE5 and KRE6 was successful in the dimorphic fungus Cryptococcus neoformans, attempts to delete these genes in C. graminicola failed (FIG. 12). PCR screens performed with more than 200 single-spore isolates of transformants uniformly led to amplification of a 4.8 and 2.2 kb fragment indicative of the intact KRE5 and KRE6 genes, respectively (FIG. 12B), and of a 1.7 and 1.5 kb fragment, suggesting that the deletion cassettes carrying the Nourseothricin (Nat) or Geneticin resistance gene (G418) had integrated ectopically. As average rates of homologous integration in C. graminicola are usually above 30%, these results strongly suggest that deletion of KRE5 or KRE6 is lethal.

To circumvent the lethality of KRE5 and KRE6 deletion, a RNAi-based knock down strategy was adopted, comparable to the strategy used to down-regulate GLS1 transcript concentrations in C. graminicola (Kück and Hoff, 2010, Appl. Microbiol. Biotechnol. 86: 51-62) (FIG. 13 and FIG. 14). The RNAi vector consisted of the trpC promoter of Aspergillus nidulans, 928 bp and 923 bp sense and antisense fragments of the second exon of the KRE5 and KRE6 genes of C. graminicola, respectively, separated by 135 bp of the second intron of the Cut2 gene of M. oryzae, followed by the trpC terminator of A. nidulans. The Nourseothricin resistance gene from Streptomyces noursei (Malonek S, et al., 2004, J. Biol. Chem. 279: 25075-25084) was used as the selection marker (FIG. 14A).

XhoI-digested genomic DNA of the WT and RNAi strains was analyzed by Southern hybridization. Ten KRE5- and six KRE6-RNAi strains harboring single or two copies of the RNAi construct in their genome have been identified (FIGS. 14B and C). The RNAi strains showed gradually and significantly reduced KRE5 and KRE6 transcript abundance, as indicated by RT-qPCR analyses. As compared with the WT strain, KRE5-RNAi strains showed KRE5 transcript abundances between 53 and 10%, and KRE6 transcript abundances in KRE6-RNAi strains ranged between 51 and 19%, respectively (FIGS. 13A and B). All RNAi strains exhibited severely reduced growth rates (FIGS. 13C and D).

Furthermore, analysis of hyphal penetration competence was performed in race tubes containing different agar concentrations (Brush and Money, 1999, Fungal Genetics and Biology 28: 180-200). All RNAi strains had severe invasive growth defects, and the severity of the growth defect correlated with the KRE5 and KRE6 transcript abundance (FIGS. 13C and D, right penal). Intriguingly, light microscopy revealed that vegetative hyphae of both KRE5- and KRE6-RNAi strains exhibited severe hyphal swellings (FIGS. 13E-G), many of which contained intrahyphal hyphae reminiscent of mutants of several fungi deficient in class V chitin synthase (FIGS. 13F and G, insert, asterisks). Comparable to GLS1-RNAi strains (Oliveira-Garcia and Deising, 2013), hyphal swellings were strongly pigmented (FIGS. 13F and G, insert, black arrowheads), but in contrast to these, vegetative hyphae of KRE5- and KRE6-RNAi strains often burst to released lipid vesicles (FIGS. 13F and G, arrows; FIG. 13H).

In order to investigate whether reduction of transcript abundance led to reduced Kre5 and Kre6 protein contents, the KRE5 and KRE6 genes of the WT strain and of KRES5:RNAi and KRE6:RNAi strains were replaced by the KRES5:mCherry or KRE6:mCherry construct. Two representative KRES5:mCherry or KRE6:mCherry replacement strains were comparatively analyzed by quantitative fluorescence microscopy (FIGS. 13I and K). Vegetative hyphae of the two RNAi strains showed a reduction of mCherry fluorescence by 64.4±12.9% and 68.6±15.1% in KRE5-RNAi strains (FIG. 13I), and 59.3±16.8% and 62.4±11.9% in KRE6-RNAi strains (FIG. 13K), clearly indicating that not only transcript abundance, but also Kre5 and Kre6 protein concentrations were down-regulated in the RNAi strains. To analyze differences between alkali-soluble β-1,6-glucan levels (Gilbert N M et al., 2010) in cell walls of the C. graminicola WT and RNAi strains, alkali-soluble β-1,6-glucan of these strains was spotted onto nylon membranes and incubated with the β-1,6-GBP:YFP probe (FIG. 9). Although this method determines the total β-1,6-glucan contents in the cell walls only semi-quantitatively, it clearly showed that β-1,6-glucan contents were severely reduced in KRE5- and the KRE6-RNAi strains, as compared with the WT strain (FIG. 13L).

As the phenotypes of the KRE5- and KRE6-RNAi strains had striking similarities with those of GLS1-RNAi strains, the GLS1 transcript levels and β-1,3-glucan contents were also evaluated in KRE5- and KRE6-RNAi strains. Indeed, the GLS1 transcript levels were reduced in vegetative hyphae of KRE5- and KRE6-RNAi strains (FIG. 13M), and so were the β-1,3-glucan contents, as indicated by aniline blue fluorochrome fluorescence quantification (FIG. 13N) (Oliveira-Garcia and Deising, 2013) .

These data show that KRE5 and KRE6 play essential roles in formation of β-1,6-glucan bonds, that β-1,3- and β-1,6-glucan synthesis are co-regulated and that the synthesis of a β-1,3-glucan network is indispensable for cell wall function in vegetative hyphae of C. graminicola. By reduction of KRE5 and KRE6 expression, the GLS1 expression is hampered and, besides β-1,6-glucan synthesis, also β-1,3-glucan synthesis is disturbed. This is a technical improvement over the targeted reduction of the GLS1 transcription (Oliveira-Garcia and Deising, 2013).

KRE5 and KRE6 of C. graminicola are Required for Asexual Sporulation, Adhesion, and Differentiation of Functional Appressoria

Efficient asexual sporulation is a fungal hallmark and indispensable for spreading of fungal plant diseases. Intense fluorescence of conidia of KRE5:mCherry or KRE6:mCherry replacement strains and intense labeling by the β-1,6-GBD:YFP-probe (FIG. 10) suggested that expression of KRE5 and KRE6 and synthesis of β-1,6-glucan is required for formation of conidia. Indeed, reduction of KRE5 and KRE6 transcript abundance by RNAi strongly reduced asexual sporulation rates, irrespective of the addition of osmolytes to the growth medium (FIG. 9A). Moreover, conidial size and shape (FIGS. 9B, C, F, and I) were strongly affected, and coalesced cytoplasm was visible in conidia of KRE5- and KRE6-RNAi strains (FIGS. 9D and G, arrows). In addition, conidia of the RNAi strains frequently burst and released lipid vesicles (FIGS. 9E and H, and FIG. 9K). Increased rates of conidial bursts in the RNAi strains further suggested that conidial cell wall rigidity is affected by down-regulation of KRE5 and KRE6 transcript abundance. Resistance to cell wall-degrading enzymes is a valid criterion for cell wall rigidity (Oliveira-Garcia and Deising, 2013), and we therefore incubated conidia with cell wall lyzing enzymes from Trichoderma harzianum and measured rates of protoplast formation. Clearly, conidia of KRE5- and KRE6-RNAi strains protoplasted faster than those of the WT strain (FIG. 9L), confirming the hypothesis of cell wall defects in RNAi strains. Introduction of cell wall defects by treatment of the mammalian pathogen Candida albicans with the β-1,3-glucan synthase inhibitors, i.e. echinocandin fungicides, caused up-regulation of chitin synthases as a compensatory response (Walker et al., 2008, PLoS Pathogens 4: e1000040). In conidia of KRE5- and KRE6-RNAi strains of C. graminicola CHSV transcript abundance was significantly increased, supporting the idea that cell wall defects exist in conidia of these strains (FIG. 9M). GLS1 transcript levels, in contrast, were strongly reduced in both KRE5- and KRE6-RNAi strains (FIG. 9M), again suggesting co-regulation of β-1,3- and β-1,3-glucan synthesis (see FIGS. 13M and 13N).

RNAi strains exhibiting KRE5 or KRE6 transcript abundance of less than 25% did not sporulate under any of the growth conditions tested.

When osmotically stabilized, conidia of the WT and RNAi strains germinated and differentiated appressoria on artificial substrata (FIG. 15). Intriguingly, while germ tubes of the WT strain were non-melanized and short, 80% and 74% of the germ tubes formed by KRE5- and KRE6-RNAi strains, respectively, were swollen and/or melanized (FIG. 15A, KRE5-RNAi, lower micrographs, arrowheads; FIG. 15B). While appressoria of the WT strain formed on polyethylene sheets did not develop further and remained unaltered, approx. 97% of the appressoria formed by KRE5-RNAi strains and 75% of the appressoria formed by KRE6-RNAi strains ruptured spontaneously (FIG. 15A, upper micrographs, short black arrows; FIG. 15C), released lipid droplets, (FIG. 15A, KRE5- and KRE6-RNAi, long black arrows), and collapsed (FIG. 15A, KRE5- and KRE6-RNAi, upper micrographs, asterisks).

Interestingly, while the vast majority of the KRE5-RNAi strains released cellular components (FIG. 15A, KRE5-RNAi, upper micrograph, arrow) preferentially at the germ tube-appressorium border (FIG. 15A, KRE5-RNAi, upper micrograph, arrowhead), approx.15% of the appressoria of KRE6-RNAi strains released droplets through the entire appressorial cell wall, possibly due to non-rigidified cell walls with increased pore sizes (FIG. 15A, KRE6-RNAi, lower left micrograph, arrowheads).

As tight adhesion of appressoria is required for direct penetration of the plant cell wall, efficient adhesion is essential for full virulence. This is particularly true for pathogens that invade their hosts primarily by exertion of force. As compared with the WT strain, adhesion of infection structures of KRE5-RNAi strains 1-3, and of KRE6-RNAi strains 1 and 2 was reduced by more than 95% on all surfaces tested, i.e. onion epidermal cells, polyester and glass (FIG. 15D).

Interestingly, also appressorial cell wall elasticity was affected by down-regulation of KRE5 and KRE6 transcript abundance (FIG. 16). When KRE5-RNAi and KRE6-RNAi strains formed appressoria in sterile distilled water, the vast majority of these cells ruptured (FIGS. 15A and C). The minor fraction of appressoria that did not explode allowed analyzing the rigidity of the appressorial cell wall by measuring appressorial diameters in solutions differing in osmotic potential (FIG. 16). In distilled water, both KRE5- and KRE6-RNAi strains showed significantly increasing appressorial diameters. After addition of the osmolyte PEG6000 (400 mg/mL), however, appressoria shrunk by approx. 50% and reached the diameter of WT appressoria, indicating that appressorial cell walls of the RNAi strains are highly elastic. The appressorial diameter of the WT strain was unaffected by the osmotic potential of their environment (FIGS. 16A and B). Increased appressorial cell wall elasticity is likely to reduce the appressorial turgor pressure, as shown previously for appressoria of C. graminicola GLS1-RNAi strains. Indeed, WT appressoria showed incipient cytorrhizis at a PEG6000 concentration of more than 400 mg/mL, whereas a concentration of approx. 100 mg/mL was sufficient in the KRE5- and KRE6-RNAi strains (FIG. 16C), indicating that mutants affected in β-1,6-glucan synthesis had severely reduced appressorial turgor pressure.

The data shown here indicate that formation of β-1,6-glucosidic bonds is required for germination, adhesion of infection cells, cell wall rigidity, and for generation of appressorial turgor pressure.

KRE5 and KRE6 are Required for Appressorial Penetration, Invasive Growth and Pathogenicity on Maize Leaves

In order to investigate the role of KRE5 and KRE6 of C. graminicola in plant infection, conidia of the WT strain and of KRE5- and KRE6-RNAi strains were inoculated onto intact and wounded segments of the youngest fully expanded leaf of two- to three-week-old maize plants, and virulence was evaluated seven days after inoculation (DAI). The WT strain caused severe disease symptoms on both intact and wounded leaves, but none of the RNAi strains tested was able to invade intact leaves (FIG. 17A, non-wounded). These data were not surprising, as appressorium function was strongly affected in KRE5- and KRE6-RNAi strains (FIGS. 15 and 16). To circumvent appressorial defects and to analyze host tissue invasion, we performed wound inoculation experiments. On wounded leaves the RNAi strains caused minor necroses at the margins of the wounds (FIGS. 17A, wounded). Assessment of fungal development by quantitative PCR (qPCR), using internal transcribed spacer two (ITS2) primers (Behr et al., 2010, Molec. Plant-Microbe Interact. 23: 879-892), fully confirmed the macroscopically observed virulence defects of KRE5- and KRE6-RNAi strains (FIG. 17B).

Differential interference contrast (DIC) microscopy was employed to characterize the role of KRE5 and KRE6 in wound colonization of C. graminicola. The WT strain formed fast growing thin necrotrophic hyphae immediately after wound inoculation, without establishing a biotrophic interaction (FIG. 17C, WT, arrowheads). When KRE5-RNAi or KRE6-RNAi strains were inoculated onto wounded leaves, slowly growing hyphae with brown swellings formed in host cells (FIG. 17C, KRE5-RNAi and KRE6-RNAi, asterisks). The hyphal swellings in both RNAi strains were connected by thin hyphal segments (FIG. 17C, KRE5-RNAi and KRE6-RNAi, arrowheads, comparable to those seen in GLS1-RNAi strains (Oliveira-Garcia and Deising, 2013). In spite of the distinct cell wall defects, hyphae of KRE5- and KRE6-RNAi strains were able to penetrate the anticlinal maize cell walls (FIG. 17C, KRE5-RNAi and KRE6-RNAi, arrows). Interestingly, both RNAi strains grew preferentially in close contact with the maize cell walls. By contrast, infection hyphae of the WT strain were thin and grew across the cells and only rarely in association with the host cell wall (FIG. 17C).

Dramatic biotrophy-specific down-regulation of β-1,6-glucan synthesis (FIG. 10) perfectly mirrors the profile of β-1,3-glucan synthesis in infection structures of C. graminicola (Oliveira-Garcia and Deising, 2013). Overexpression of GLS1 in biotrophic hyphae and exposition of β-1,3-glucan on surfaces of these cells led to dramatic defense responses in maize (Oliveira-Garcia and Deising, 2013), raising the question whether overexpression of KRE5 and KRE6 would likewise activate defense responses. In order to address this question, we constructed mutants overexpressing these two β-1,6-glucan synthesis genes under the control of the strong constitutive trpC promoter of Aspergillus nidulans (FIG. 18A). Two mutants with single integration events of the KRE5 and KRE6 overexpression constructs were identified and used in further studies (FIG. 18B). The mutants exhibited significantly increased KRE5 and KRE6 transcript abundances (FIG. 18C) and showed growth rates comparable to that of the WT strain (FIG. 18D).

Intriguingly, while the PtrpC:KRE5 and PtrpC:KRE6 overexpression strains caused severe disease on wounded maize leaves, they were unable to evoke anthracnose disease symptoms on intact leaves. By contrast, both WT and PtrpC:KRE5 and PtrpC:KRE6 overexpression strains exhibited full virulence on wounded leaves (FIG. 19A). Pathogenicity defects of the KRE-overexpressing strains on intact leaves was fully confirmed by qPCR analyses (FIG. 19B). The inability to cause disease was surprising because both, PtrpC:KRE5 and PtrpC:KRE6 strains efficiently formed appressoria on the intact plant cuticle, invaded the host epidermal cell and formed an infection vesicle (FIG. 19C, non-wounded, ap and iv). Excitingly, while the WT strain formed primary hyphae without eliciting visible plant defense responses (FIG. 19C, non-wounded, WT, iv and ph), massive formation of darkly pigmented vesicles was observed in plant cells infected by PtrpC:KRE5 and PtrpC:KRE6 strains, and these vesicles associating with the invading infection vesicle and may be causal for growth arrest of the overexpression strains at this early stage of the infection (FIG. 19C, non-wounded, PtrpC:KRE5 and PtrpC:KRE6 strains, arrows). Interestingly, also the hyphae of PtrpC:KRE5 and PtrpC:KRE6 strains invading the host tissue from wounds were decorated with dark vesicles (FIG. 19C, wounded, PtrpC:KRE5 and PtrpC:KRE6, arrows). In contrast, these vesicles were not seen in wounded leaves infected by the WT strain (FIG. 19C, wounded, WT).

To further characterize plant defense responses at the cellular/histological level, cell wall responses, including formation of papillae, were analyzed by UV-fluorescence microscopy and 3,3′-diaminobenzidine (DAB) staining. Both WT as well as KRE5 and KRE6 overexpressing strains formed large numbers of dark melanized appressoria on the plant surface (FIG. 19D, arrows). However, only the appressoria of the PtrpC:KRE5 and PtrpC:KRE6 strains were decorated by brightly fluorescing papillae (FIG. 19D, UV, PtrpC:KRE5 and PtrpC:KRE6, arrowheads), and occasionally whole cell fluorescence was observed (FIG. 19D, UV, PtrpC:KRE6, asterisk). Quantification of papilla-based defense responses showed that less than 10% of the WT appressoria provoked papilla formation, whereas 49.8 to 89.6% of the appressoria of the PtrpC:KRE5 and PtrpC:KRE6 strains were decorated by plant cell wall appositions (FIG. 19E). Furthermore, PtrpC:KRE5 and PtrpC:KRE6 strains evoked a strong H202 response, as visualized by massive browning after DAB staining (FIG. 19D, DAB, PtrpC:KRE5 and PtrpC:KRE6). In cells infected by the WT strain these responses did not occur (FIG. 19D, DAB, WT).

These data strongly suggest that overexpression of KRE5 and KRE6 during the biotrophic phase of the interaction between C. graminicola and maize leads to exposition of β-glucan on the hyphal surface and to induction of defense responses. Induction of β-glucan-induced defense responses have been observed after overexpression of GLS1 in biotrophic hyphae of this fungus, and RNAseq analyses helped identifying genes putative defense-related genes on a genome-wide level (Oliveira-Garcia and Deising, 2013). Candidate genes identified included genes encoding classical PR proteins such as PR1, a number of different PR2 proteins, i.e. β-1,3-glucanases, a PR3 protein representing a chitinase, as well as PR5. Furthermore, plant cell wall response genes encoding cellulose synthase-like proteins and peroxidases, as well as genes encoding four different terpene synthases, putatively involved in phytoalexin synthesis, have been identified. As these genes have been identified as β-glucan-triggered genes, we analyzed transcript abundance of these genes in maize plants inoculated with the WT C. graminicola strain as well as strains overexpressing KRE5 and KRE6 during the biotrophic phase of pathogenesis, taking advantage of RT-qPCR (FIG. 19).

Indeed, all of the 18 putative defense genes tested were significantly up-regulated after inoculation with the PtrpC:KRE5 and PtrpC:KRE6 strains, as compared to the plants inoculated with the WT strain and with mock-treated plants. While PR1 transcripts were 9.1- and 8.3-fold increased in plants inoculated with PtrpC:KRE5 and PtrpC:KRE6 strains, respectively, different PR2 genes were up-regulated between 10- and 100-fold. The thaumatin-like PR5, which is thought to bind to β-1,3-glucan, was up-regulated 11.5- and 9.2-fold after inoculation with PtrpC:KRE5 and PtrpC:KRE6 strains, respectively. Remarkably, the CTB gene, the callose synthase-like genes CS45 and CS31, as well as the peroxidase gene PX82 were up-regulated more than 250-fold. Furthermore, the four terpene synthase genes TPS2, TPS3, TPS7, and TPS10 were up-regulated between 40- and 170-fold in plants inoculated with strains synthesizing β-1,6-glucan in the biotrophic phase of infection (FIG. 19).

Collectively, these data show that the β-1,6-glucan biosynthesis genes KRE5 and KRE6 are required for differentiation of functional infection structures and that biotrophy-specific down-regulation of synthesis of branched β-glucan in the hemibiotroph C. graminicola is required for evading PAMP-triggered plant immune responses.

The cell wall of an infecting fungal hypha is the first structure establishing a physical contact with the host plant. Host tissue invasion requires highly coordinated synthesis of various cell wall polymers at the hyphal apex and sub-apical cross-linking of polymers, leading to formation of the rigid scaffold responsible for shape and structural integrity of the hypha. However, in plant pathogens maintenance of hyphal structural rigidity is mediated by invariable cell wall polymers, collectively referred to as pathogen-associated molecular patterns (PAMPs). At host invasion, the pathogen is confronted by the dilemma that, on one hand synthesis of structural cell wall polymers is indispensable, and on the other hand PAMP exposure bears the risk of recognition of pathogen attack by the plant. So far, primarily the role of major fungal cell wall polymers in pathogenesis has been studied in plant pathogenic filamentous fungi. Others, which may represent quantitatively minor, but possibly functionally important polymer fractions, have so far been disregarded. Here we show that KRE5 and KRE6, likely needed for introduction of β-1,6-branches into β-1,3-glucan polymers and for cross-linking of polymers, are required for functional integrity of vegetative and pathogenic hyphae, and that infection structure-specific control of expression of these genes is indispensable for the establishment of a compatible parasitic interaction between C. graminicola and maize.

Structural Integrity of the Cell Wall is Essential for Vegetative Growth, Infection Structure Function and Host Tissue Invasion

Remarkably, down-regulation of transcript abundance of KRE5, KRE6, and of GLS1 by RNAi resulted in comparable phenotypes, all of which were very severe. For example, reduced growth rates and hyper-pigmented swellings were observed in vegetative hyphae (FIG. 13), and conidiation rates were strongly reduced, with severely misshapen conidia formed (FIG. 9). Furthermore, appressoria of RNAi strains were unable to control turgor pressure and burst (FIG. 15), and infection hyphae exhibited swellings and were unable to cause clear disease symptoms (FIG. 17). Taken together, these data indicate that the β-1,3-β-1,6-glucan network is indispensable at all major stages of fungal development, including pathogenesis.

In conidia, a complex structural network of polysaccharides defines the shape of the spore and protects it from detrimental compounds produced by bacteria and fungi of the phylloplane. In addition, in plant pathogenic fungi the cell wall mediates adhesion of spores to the cuticle of the host (FIG. 15D; see (Oliveira-Garcia and Deising, 2013). In Aspergillus nidulans, analyses of transcriptional changes occurring during the transition from hyphal growth to conidiation showed that ˜20% of all genes were significantly altered at the transcriptional level. Intriguingly, several of the top 20 up-regulated genes encode enzymes involved in cell wall biogenesis. Moreover, not only mutants carrying direct defects in cell wall biogenesis genes, but also iron uptake mutants of C. graminicola, i.e. the siderophore mutants Δsid1, Δnps6, and the ferroxidase mutant Δfet3-1, show significant sporulation deficiencies, likely due to erroneous regulation of cell wall biogenesis genes. These data suggest that conidiation requires complex regulation of a large number of genes, only some of which are directly incolved in cell wall biogenesis. It is important to note that in plant pathogenic fungi the ability to sporulate is a major factor not only determining spread of disease in the field, but also in disease initiation, as infection cells like appressoria are directly derived from spores in most cases.

As appressorial cell walls need to control significant turgor pressure, it is conceivable that the rate of appressorium differentiation and function is severely affected in KRE5- and KRE6-RNAi strains. Importantly, more than 90% and approx. 80% of the appressoria formed by KRE5-and KRE6-RNAi strains, respectively, exploded (FIG. 8C). For comparison, approx. 15% of the appressoria formed by class I GLS1-RNAi strains ruptured (Oliveira-Garcia and Deising, 2013). Class I GLS1-RNAi strains had GLS1 transcript abundances reduced by ca. 35-45%, which is comparable to the reduction in transcript concentration in KRE5- and KRE6-RNAi strains (FIGS. 6A and B, and (Oliveira-Garcia and Deising, 2013). The fact that appressorium function was strongly affected in these strains is suggestive of the role of β-1,6-bonds in cross-linking of cell wall polymers and in control of the appressorial turgor pressure. However, this interpretation must be treated with caution, as transcript concentrations and fluorescence staining of β-1,3-glucan by aniline blue fluorochrome (Oliveira-Garcia and Deising, 2013) and of β-1,6-glucan linkages by the β-1,6-GBP:YFP probe do not directly prove the existence of chemical β-1,3-β-1,6-links. As shown for A. fumigatus, enzymatic digestions with purified recombinant endo-cleaving β-1,3-glucanases and chitinases, fractionation of cleavage products by high performance liquid chromatography, and analysis of the chemical structures of and chemical bonds between the products by NMR spectrometry and GCMS clearly indicated the introduction of β-1,6-branches into linear β-1,3-glucanchains (Fontaine et al., 2000, J. Biol. Chem. 275: 27594-27607). Comparable studies would also be suited to answer the question whether β-1,6-bonds are restricted to branching points of β-1,3-strands, or whether short β-1,6-linked glucan oligomers exist in cell walls of C. graminicola. However, the occurrence of such bonds is difficult to analyze in individual infection cells such as appressoria or in planta differentiated infection hyphae, due to several reasons. First, C. graminicola does not differentiate infection structures synchronously, so that samples of infected leaves would contain mixtures of different types of hyphae. Second, chemical analysis requires cell wall masses which can be produced when vegetative hyphae are to be analyzed, but not when cell walls of specific infection structures are of interest. Third, some infection structures, such as appressoria or infection hyphae of KRE5- and KRE6-RNAi strains are melanized or contain other pigments, which may interfere with the analyses performed by Fontaine et al., 2000. Thus, although fluorescence labeling with polymer-specific probes may not provide full chemical details of the cell wall structure, it is sufficient to show whether certain polymers or bonds exist in specific infection structures and at specific stages of pathogenesis.

β-1,6-glucan-mediated cross-linking of cell wall polymers likely is a general key-process in appressorium function and initiation of host invasion, and not restricted to fungi differentiating melanized appressoria. Recent work with a polyketide synthase 1 (Pks1) deficient mutant of C. graminicola has shown that the appressorial turgor pressure does not require melanization (Ludwig et al., 2014, Mol. Plant-Microbe Interact. 27:315-327). Furthermore, turgor pressure and penetration competence of the Asian soybean rust fungus Phakopsora pachyrhizi, which penetrates its host directly through non-melanized appressoria, was determined by transmitted light double-beam interference Mach-Zehnder microscopy, in combination with incipient cytorrhysis experiments. The turgor pressure generated in the appressoria of this fungus corresponded to 5.13 MPa (Loehrer et al., 2014, New Phytol. 203: 620-631), which is similar to a turgor pressure of 5.35 MPa generated by melanized appressoria of C. graminicola (Bechinger et al., 1999, Science 285: 1896-1899).

Interestingly, also the defects in infection hyphae formed in the host cell by KRE5- and KRE6-RNAi strains resemble those of GLS1-RNAi strains (FIG. 10C, compare with (Oliveira-Garcia and Deising, 2013). Hyphal swellings and brown pigmentation clearly indicate cell wall defects, likely due to inappropriate cross-linking of polymers. An interesting though unexplained observation was that infection hyphae of KRE5- and KRE6-RNAi strains grew in close contact with the host cell wall (FIG. 10C).

Infection Structure-Specific Regulation of PAMP Exposition is Indispensable for Escaping Immune Responses

Synthesis of structurally invariable cell wall polymers is indispensable during fungal pathogenic development. Therefore, in order to recognize pathogen attack, plants have evolved plasma membrane-localized pattern recognition receptors (PRRs), the activation of which by PAMPs evokes broad spectrum immune responses (Nurnberger et al., 2004, Immunol, Rev. 198: 249-266; Muthamilarasan and Prasad, 2013, J. Biosci. 38: 1-17). For example, chitin-derived 13-1,4-N-acetyl glucosamine oligomers are recognized by the two LysM receptors CEBiP and OsCERK1 in rice (Shimizu et al., 2010, Plant J. 64: 204-214), and sub-nanomolar concentrations of N-acetyl-chitooligosaccharides are sufficient to induce plant defense responses in tomato (Felix et al., 1993, Plant J. 4: 307-316). Using soybean membranes, ligand displacement assays performed with a ¹²⁵J-labeled glucan elicitor released from mycelial walls of the oomycete Phytophthora megasperma f.sp. glycinea yielded a 50% inhibitory concentration for branched β-1,3-1,6-β-glucans of 3 nM (Cosio et al., 1990, FEBS Lett. 271: 223-226).

In order to escape the dilemma of the need of strengthening the wall of infection hyphae by structural polymers on one hand, and the necessity of avoiding PAMP recognition on the other, plant pathogenic fungi have developed an array of mechanisms in order to compromise PAMP perception. For example, the hemibiotrophic rice blast fungus M. oryzae and the biotrophic tomato pathogen Cladosporium fulvum secrete LysM domain-containing effector proteins such as Slp1, Avr4 or Ecp6, sequestering polymeric chitin exposed on hyphal surfaces or small chitin oligomers released by the activity of chitinases (de Jonge et al., 2010; Mentlak et al., 2012; van Esse et al., 2007, Mol. Plant-Microbe Interact. 20: 1092-1101). As LysM effectors are widely conserved in the fungal kingdom, sequestration of chitin may represent a common strategy of host immune evasion in many pathogens, including Colletotrichum species. Correspondingly, conversion of surface-exposed chitin to its non-acetylated derivative chitosan is specifically initiated at host invasion by the broad bean rust fungus Uromyces fabae, the wheat stem rust Puccinia graminis as well as the maize anthracnose fungus C. graminicola. Deacetylation of surface-localized chitin is likely to compromise recognition of the fungal attack, as chitosan is a poor chitinase substrate and as chitosan fragments exhibit lower elicitor activity than chitin fragments. Masking of hyphal surfaces may also be accomplished by apposition of molecules either lacking or showing reduced PAMP activity. For example, apposition of polymeric α-1,3-glucan protects infection hyphae of M. oryzae form chitinase and β-1,3-glucanase attack and thus interferes with PAMP production. Accordingly, transgenic rice plants expressing the α-1,3-glucanase gene of Bacillus circulans showed increased resistance against the ascomycetes M. oryzae and Cochlioborus miyabeanus, as well as the basidiomycete Rhizoctonia solani (Fujikawa et al., 2012, PLoS Pathog. 8: e1002882).

β-glucans are considered to be conserved across different classes of microorganisms, including fungi and oomycetes, and fragments of this polymer, like chitin fragments, represent potent PAMPs. Interestingly, Klarzynski et al., 2000, Plant Physiol. 124: 1027-1038 have shown that laminarin, a linear β-1,3-glucan from the brown alga Laminaria digitata, elicits defense responses in tobacco. Studies including laminarin, high molecular weight β-1,3-β-1,6-glucan, as well as different β-1,3-glucan oligomers indicated that defense responses were elicited by linear β-1,3-glucan oligomers, with a penta-glucan being the smallest active molecule.

Transcriptional Silencing Activity in HIGS Corn Lines

Transformation of corn with the plant transformation vector p7U-ubi RGA2intronII HIGS GLRG 05611 allowed the identification of HIGS corn lines which showed a transcriptional silencing activity against the KRE5 gene of C. graminicola (GLRG_05611 gene). In comparison to the inbred line A-188, which was used for corn transformation, the transgenic corn lines M-T-001, M-T-003, M-T-005, M-T-006 and M-T-024, which were transformed with p7U-ubi_RGA2intronII_HIGS_GLRG_05611, showed a reduction of reporter gene activity caused by siRNAi mediated degradation of the reportergene construct pABM_ubiluci_GLRG_05611 (FIG. 21). A transgenic RNAi control line, which was transformed with a RNAi construct targeting another fungal gene, revealed no silencing activity against the GLRG_05611 gene.

Enhanced Resistance of the HIGS Corn Lines M-T-001, M-T-003, M-T-005, M-T-006 and M-T-024

Resistance assays on leaf segments of maize (Zea maize) plants to show enhanced fungal resistance could be done as described (Oliveira-Garcia E, Deising H B (2013).

Fungal Strains, Culture Conditions, Infection Structure Differentiation and Virulence Assays

The wild-type (WT) strain M2 of C. graminicola (Ces.) G. W. Wilson (teleomorph Glomerella graminicola D. J. Politis) and RNAi strains generated in this study were cultivated on oat meal agar (OMA; Werner et al., 2007), complete medium (CM; Leach et al, 1982, Journal of General Microbiology 128: 1719-1729), potato-dextrose (PD; Difco Laboratories, Sparks, Md., USA), synthetic minimal medium (SMM; 10 g glucose; 1 g Ca(NO3)2; 0.2 g KH2PO3; 0.25 g MgSO4 and 0.054 g NaCl per L) or synthetic complete medium (SCM without amino acids; Becton Dickinson, Sparks, Md., USA), with amino acids added as described (Treco and Lundblad, 1993, Basic techniques of yeast genetics, New York: John Wiley & Sons). To grow RNAi strains, the media were supplemented with 0.15 M KCl, 1 M sorbitol, or 0.5 M sucrose.

Incubation of liquid and solidified media were as described (Oliveira-Garcia and Deising, 2013).

The Saccharomyces cerevisiae reference strain Y00000 (parental S288C) (Mat a, his3D1, leu2D0, met15D0, ura3D0), the Δkre5 mutant Y21633 (BY4743; Mat a/a; his3D1/his3D1; leu2D0/leu2D0; lys2D0/LYS2; MET15/met15D0; ura3D0/ura3D0; YOR336w::kanMX4/YOR336w) and the Δkre6 mutant Y05574 (BY4741; Mat a; his3D1; leu2D0; met15D0; ura3D0; YPR159w::kanMX4) (Euroscarf, Frankfurt, Germany) were grown at 30° C. and 150 rpm in liquid Yeast Extract Peptone Dextrose or Yeast Extract Peptone Dextrose Sorbitol (YPD/YPDS; Difco, Sparks, Md., USA.) lacking uracil. S. cerevisiae cells producing the yellow fluorescing β-1,6-glucan-binding protein were grown on yeast synthetic complete medium (YSCM) (Difco, Sparks, Md., USA). Solidified media contained 1.5% (w/v) agar agar (Difco, Sparks, Md., USA). Calcofluor White and killer toxin K1 were added to a concentration of 50 μg/mL.

In vitro infection structure differentiation and virulence assays on leaf segments of maize (Zea maize cv Mikado) plants or on epidermal cell layers from onion (Allium cepa cv Grano) bulbs were done as described (Oliveira-Garcia and Deising, 2013).

All experiments were performed in triplicate and with four repetitions.

Complementation of the S. cerevisiae Δkre5 and Δkre6 Mutant with the C. graminicola KRES and KRE6 cDNA

In order to synthesize cDNA of KRE5 and KRE6, total RNA was extracted (Chirgwin et al, 1979, Biochemistry 18: 5294-5299) from vegetative mycelium of C. graminicola. mRNA was isolated using the Nucleotrap mRNA Purification Kit, and cDNA synthesis was performed using the Creator SMART cDNA Library Construction Kit (BD Biosciences Clontech, Heidelberg, Germany). The primers CgKRE5Sfil-Fw and CgKRE5Sfil-Rv, CgKRE6Sfil-Fw and CgKRE6Sfil-Rv have been used to amplify the KRE5 and KRE6 cDNA, which was cloned into the SfilA-B sites of the yeast cDNA expression vector pAG300 (www.addgene.org; (Oliveira-Garcia and Deising, 2013). These primers, and others mentioned here, are listed in Table 1. Correct orientation of the DNA was confirmed by sequencing, using primer pAG300-Fw. The empty vector (pAG300) and the vectors containing the KRE5 and KRE6 cDNA were transformed into S. cerevisiae strains Y21633 and Y05574, respectively, using the lithium acetate procedure (Becker and Lundblad, 2001, Introduction of DNA into yeast cells. In: Ausubel F M, Brent R, Kingston R E, Moore D D, Seidman J G et al., editors. Curr Protoc Mol Biol. New York: John Wiley & Sons. pp. 13.17.11-13.17.10) to yield the complemented yeast strains Δkre5 (KRE5) and Δkre6 (KRE6). As control, strains Y21633 and Y05574 were also transformed with empty pAG300, yielding Δkre6 (pAG300) and Δkre6 (pAG300). Yeast cells were grown on YSCM agar lacking uracil.

Targeted deletion, promoter exchange and overexpression of KRE5 and KRE6, construction of RNAi strains and generation of C. graminicola KRES5:mCherry and KRE6:mCherry replacement strains

For targeted deletion of the 4581-bp C. graminicola KRE5 gene, the Nourseothricin acetyl-transferase gene Nat-1 from Streptomyces noursei was PCR-amplified from pNR1 (Malonek et al., 2004), using primers Nourse1pNR1-Fw, and Nourse1pNR1-Rv. The 1022-bp 5′ and the 1007-bp 3′ flanking regions of the KRE5 gene were amplified from genomic DNA, using primers CgPKRE5-fw, CgP1KRE55′-flank-ry and CgTKRE53′-flank-fw and CgTKRE5-rv, respectively. The products were fused by joint-PCR (Yu et al., 2004, Fungal Genet. Biol. 41: 973-981), and nested primers CgPKRE5nest-fw and CgTKRE5nest-ry were used to amplify the 4210-bp KO construct, which was transformed into conidial protoplasts (Werner et al., 2007). Tests for homologous integration of the KO construct was done with primers CgPKRE5test-fw and CgTKRE5test-rv.

For targeted deletion of the 1490-bp C. graminicola KRE6 gene, the Nourseothricin acetyl-transferase gene Nat-1 from Streptomyces noursei was PCR-amplified from p1199 (Namiki et al., 2001, Mol. Plant-Microbe Interact. 14: 580-584), using primers Gen1pNR1-Fw and Gen1pNR1-Rv. The 998-bp 5′ and the 1003-bp 3′ flanking regions of the KRE6 gene were amplified from genomic DNA, using primers CgPKRE6-fw, CgP1KRE65′-flank-ry and CgTKRE63′-flank-fw and CgTKRE6-rv, respectively. The products were fused by joint-PCR (Yu et al., 2004), and nested primers CgPKRE6nest-fw and CgTKRE6nest-ry were used to amplify the 4210-bp KO construct, which was transformed into conidial protoplasts (Werner et al., 2007). Tests for homologous integration of the KO construct was done with primers CgPKRE6test-fw and CgTKRE6test-rv.

The RNAi cassette from plasmid pRedi (Janus et al., 2007, Appl. Environ. Microbiol. 73: 962-970) was used to generate an RNAi constructs targeting KRE5 and KRE6 transcripts, respectively. The 910-bp KRE5 and 926-bp KRE6 sense and antisense fragments were amplified from genomic DNA of C. graminicola, using the primers RNAi(KRE5)-fw and RNAi(KRE5)-Rv, RNAi(KRE5)i-fw and RNAi(KRE5)i-Rv, RNAi(KRE6)-fw and RNAi(KRE6)-Rv, and RNAi(KRE6)i-fw and RNAi(KRE6)i-Rv. The sense and antisense fragments were used to replace the XhoI-SnaBI and BgIII-ApaI fragments of pRedi, and were thus separated by 135 bp of the intron of the M. oryzae Cut2 gene (NCBI: XM_365241.1), existing in pRedi, as a linker (Janus et al., 2007). The resulting 6.75 kb and 6.85 kb RNAi constructs were excised from pRedi by DraI digestion, purified by gel elution, transformed into conidial protoplasts of C. graminicola and single spore isolates were generated (Werner et al., 2007).

To study cell-specificity of expression of KRE5 and localization of the protein, a KRES5:mCherry replacement construct, consisting of the 1 kb 3′-end of the coding region of the KRE5 gene fused in frame to the mCherry gene, followed by the Hyg^{R} resistance cassette and the 1 kb 5′-non-coding sequence of KRE5 containing the terminator, was transformed into the C. graminicola WT strain. The 5′-coding region of KRE5 was amplified with the primers CgKRESGFP-Fw and CgKRE5GFP5′-flank-Rv, using genomic DNA as the template. The mCherry gene and the Hyg^{R} cassette were amplified using primers EGFP-Fw and HygR-Rv, with plasmid pSH1.6EGFP, kindly provided by Amir Sharon, Tel Aviv University, Israel, as template. Using genomic DNA as template, the 3′-flank of KRE5 was amplified with primers CgKRE5GFP3′-flank-Fw and CgTKRE5GFP-Rv. The KRES5:mCherry construct was fused by double-joint-PCR (Yu et al., 2004), and the complete 6.2 kb fragment was amplified with nested primers CgKRE5:GFP.nest-fw and CgKRE5:GFP.nest-rv. Phusion® High-Fidelity DNA Polymerase (New England Biolabs, Ipswich, Mass., USA) was used in all PCR reactions. The KRES5:mCherry construct was transformed into conidial protoplasts (Werner et al., 2007), and single spore isolates were tested for site-specific integration and replacement of the WT KRE5 gene by Southern hybridization.

For overexpression of KRE5, the trpC promoter of A. nidulans was amplified from pSM1 (Poggeler et al., 2003, Curr. Genet. 43: 54-61), using primers PtrpC-Sac1-Fw and PtrpC-Sac1-Rv. The toxB promoter of Pyrenophora tritici-repentis was amplified from pCM29 (Andrie et al., 2005, Mycologia 97: 1152-1161), using primers PtoxB-Sac1-Fw and PtoxB-Sac1-Rv. The PCRs products were digested by SacI, purified, and ligated into SacI-digested pNR1. The complete KRE5 gene was amplified with the primers CgKRE5NotI-Fw and CgTKRE5NotI-Rv. The 7050-bp PCR product was Not!-digested, purified and ligated into pNR1. The resulting 10,032-bp (PtrpC:KRES5:NatR) construct was amplified using primes NatOvExp.nest-Fw and NourspNR1-Rv, and transformed into conidial protoplasts (Werner et al., 2007).

Single spore isolates were tested by Southern hybridization for numbers of the KRE5 overexpression constructs integrated.

Generation of a yellow fluorescing β-1,6-glucan-binding protein allowing specific detection of β-1,6-bonds in cell wall polymers

1. In order to synthesize the yellow fluorescing β-1,6-glucan-binding protein probe, the β-1,6-glucan-binding domain (nt 90 to 360 ABC) of the endo13-1,6-glucanase of C. graminicola (GLRG_00130.1) was amplified from cDNA and integrated into yeast expression vector pJR1138 (Yalovsky et al., 1997, Mol. Cell. Biol. 17: 1986-1994). The primers Cg1-6GBPEcoRI-Fw and Cg1-6GBPXhoI-Rv have been used to amplify the cDNA of the β-1,6-glucan-binding protein cDNA, which was cloned into the EcoRI and XhoI sites of pJR1138. These primers, and others mentioned here, are listed in Supporting Table 1. The empty vector and the vector containing the β-1,6-glucan-binding protein were transformed into S. cerevisiae strains Y00000, using the lithium acetate procedure (Becker and Lundblad, 2001). Yeast cells were grown on YSCM agar lacking leucine.

DNA Extraction and Genomic DNA Gel Blot Analysis

Extraction of genomic DNA and Southern blot analyses were performed as described (Werner et al., 2007). To analyze transformants for the number of integrations of the RNAi construct, 10 mg of DNA digested with the appropriate restriction endonuclease were used. The 511-bp alkali-labile DIG-dUTP-labeled probe (Roche Diagnostics, Mannheim, Germany) specific for the nourseothricin acetyl transferase gene (Nat-1) was amplified from plasmid pNRI, using primers NatR probe-Fw and NatR probe-Rv. To analyze transformants for the correct integration of a single copy of the KRES5:mCherry and KRE6:mCherry cassette into genomic DNA of WT and RNAi strains, Southern blots were hybridized with a 500-bp DIG-dUTP-labeled hygromycin phosphotransferase (Nat^{R})-specific probe, amplified from the KRES5:mCherry and KRE6:mCherry construct, using primers NatR probe-fw and NatR probe-rv.

Quantitative PCR and RT-PCR

To quantify fungal development, qPCR was performed as described (Oliveira-Garcia and Deising, 2013), with 100 nM of each primer (CgITS2-q1, CgITS2-q2). All values are standardized to the average threshold cycle value obtained with DNA extracted from non-wounded leaves inoculated with the C. graminicola wild-type strain at 0 HAI.

Quantitative RT-PCR (RT-qPCR) was performed with 100 ng of total RNA pretreated with RQ1 RNase-free DNase (Promega, Madison, Wis., USA) as described (Oliveira-Garcia and Deising, 2013). Primers used are given in Supporting Table 1 Online). Melting curve and agarose gel analyses confirmed amplification of a single product. Transcript abundance was calculated and normalized as described (Gutjahr et al., 2008, Plant Cell 20: 2989-3005), using α-actin and histone H3 for C. graminicola and histone H2B and β-tubulin for Z. mays. CT values of three independent replicates were used to calculate mean values and standard deviations.

Microscopy and Imaging

Bright-field, differential interference contrast (DIC) microscopy and fluorescence microscopy was performed using a Nikon Eclipse 600 or a Nikon Eclipse 90i confocal laser scanning microscope (Nikon, Dusseldorf, Germany). For fluorescence microscopy, a Plan Apo 60/1.4 oil lens and the following settings were used: Excitation wavelength, 488 nm; laser light transmittance, 25% (ND4 in, ND8 out); pinhole diameter, 30 mm.

To analyze β-1,6-glucan contents of cell walls, infected maize leaves were harvested at 0, 12, 24, 48, and 72 HAI and stained with β-1,6-GBP:YFP for 20 min, 23° C. To analyze β-1,3-glucan contents of cell walls, infected maize leaves were harvested at 0, 12, 24, 48, and 72 HAI and stained with Aniline Blue Fluorocrome (Biosupplies Australia Pty Ltd, Parkville Victoria, Australia) as described (Oliveira-Garcia and Deising, 2013). To exclude that differential Aniline Blue Fluorocrome staining was due to masking of β-1,3-glucan by apposition of α-glucan, specimens were incubated at 60° C. or autoclaved in 0.1 N NaOH for 20 min and subsequently stained with Aniline Blue Fluorocrome.

Quantitative fluorescence levels of KRES5:mCherry and KRE6:mCherry expressing transformants of C. graminicola were evaluated at 0, 12, 24, and 72 HAI, using a Zeiss Observer Z1 inverted microscope equipped with a Plan Apochromat 63x/1.40 oil immersion objective and an AxioCam MRm camera. Epi-illumination analyses employed filter set 49 for Aniline Blue Fluorocrome and filter set 38HE for mCherry. Image acquisition and analysis were performed by using Zeiss AxioVision 4.8.2 (06-2010) software with the Physiology module (all from Carl Zeiss, Oberkochen, Germany).

Cross sections of biotrophic and necrotrophic infection hyphae (20 pm) were made, using Plaque GP-low-melting agarose (Biozym, Hessisch Oldendorf, Germany)-imbedded specimens and a Carl Zeiss microtome (Hyrax V50; Carl Zeiss, Jena, Germany).

Other Methods

Appressorial turgor pressure was measured as incipient cytorrhizis, using polyethylene glycol 6000 (PEG 6000; 400 mg/mL) as described (Oliveira-Garcia and Deising, 2013).

Hyphal penetration rates was measured on PDA, using different agar concentrations as described (Brush and Money, 1999).

To measure conidiation, Petri dishes (0 9 cm) with 14 d old fungal cultures grown on OMA were washed on a rotary shaker with 10 mL 0.01% (v/v) Tween 20 for 10 min to yield homogenous conidial suspensions. Conidia were counted in a Thoma chamber.

Molecular Phylogenetic Analyses

Multiple sequence alignments were done using ClustalW (http://embnet.vital-it.ch/software/ClustalW.html; (Larkin et al., 2007, Clustal W and Clustal X version 2.0. Bioinformatics 23: 2947-2948). Phylogenetic dendograms were constructed using MEGA 5 (www.megasoftware.net; (Tamura et al., 2011, Mol. Biol. Evol., 28: 2731-2739), with the minimum evolution algorithms using 1000 bootstrap replications. Sequences of all fungal KRE genes were obtained from the database of the National Center for Biotechnology Information (http://www.ncbi.nlm.nih.gov/).

Statistics

Calculations (t-test, analysis of variance) were performed with the software XLSTAT version 2009.4.02 (Addinsoft, Witzenhausen, Germany).

TABLE 1
PCR-primers (SEQ ID NOs: 243-365) (in the
order of the listing of the primers)

| | | Refer- |
|---|---|---|
| Primer | Sequence [5′→3′] | ence |
| CgKRE5 | CCAGCGGCCGCATGGGCCTTGCCCT- | This |
| NotI-Fw | GACGCTGT | study |
| CgKRE5 | CAGCGGCCGCCTATAGCTCATCTATGACAT- | This |
| NotI-Rv | GCGTATT | study |
| CgKRE6S | AAAGGCCATTACGGCCATGGATCAAGAC- | This |
| filA-Fw | CAGGGCTACTAC* | study |
| CgKRE6S | AAAGGCCGCCTCGGCCTTACAACTCTTCA- | This |
| filB-Rv | TCGCCGTCAC | study |
| CgPKRE6- | TCGGCAGCTGAACGGAAAG | This |
| Fw | | study |
| CgP | GTGCAACTGACAGTCGTACAGG- | This |
| KRE65′- | TATCGAGTGGCTTGGAAC* | study |
| flank-Rv | | |
| CgTKRE | GTCTGGAGTCTCACTAGCTTGCGCGGAATT- | This |
| 63′- | GGTTCACAACGG | study |
| flank-Fw | | |
| CgTKRE6- | CCTCCGTGCAGCATGTAATC | This |
| Rv | | study |
| CgPKRE6 | AGTGCCTTGAGGGCACGTTG | This |
| nest-Fw | | study |
| CgTKRE6 | GCAACCTCGTTCGTTAGTG | This |
| nest-Rv | | study |
| Cg KRE6 | TTGGGAGGTGGCTGAACATC | This |
| test-Fw | | study |
| Cg KRE6 | ACACGTGCTCGAAGCACTAGG | This |
| test-Rv | | study |
| CgPKRE5- | TGGTCTGCTCTCACCTCTTG | This |
| Fw | | study |
| CgP | GTGCAACTGACAGTCG- | This |
| KRE55′- | TACAAAGGGCTGTCCTCCAATTTC* | study |
| flank-Rv | | |
| CgT | GTCTGGAGTCTCACTAGCTT- | This |
| KRE53′- | CTAGTCGGTCTGGCTTCTTG | study |
| flank-Fw | | |
| CgT | CGGGAAAGATGCAAGTCCTC | This |
| KRE5-Rv | | study |
| Gen1pII | TGTACGACTGTCAGTTGCACAGCGCGTT- | This |
| 99-Fw | GTTGGATTAAG | study |
| Gen1pII | AAGCTAGTGAGACTCCAGACCTCA- | This |
| 99-Rv | GAAGAACTCGTCAAGAAG | study |
| Nourse1 | TGACCGGTGCCTGGATCTTCCTATAGAATC | Oliveira- |
| pNR1-Fw | | Garcia |
| | | & |
| | | Deising, |
| | | 2013. |
| Nourse2 | GGTCGGCATCTACTCTATTCCTTTGCCCTC | Oliveira- |
| pNR1-Rv | | Garcia |
| | | & |
| | | Deising, |
| | | 2013. |
| CgPKRE5 | CGAACTGTATGCTGGGTGAG | This |
| nest-Fw | | study |
| CgTKRE5 | GTCAGGACACGAACGACAAG | This |
| nest-Rv | | study |
| Cg KRE5 | TCCAGAAGCGGCCTGCTTAC | This |
| test-Fw | | study |
| Cg KRE5 | CGTCACATTGCCTGCTGAAC | This |
| test-Rv | | study |
| RNAi | AAACTCGAGTCATGTCGGTGCGGACAATC | This |
| (KRE5)- | | study |
| Fw | | |
| RNAi | AAAAAGCTTTCTTTCAAGGGCCTGCCCTC | This |
| (KRE5)- | | study |
| Rv | | |
| RNAi | AAAAGGCCTTCTTTCAAGGGCCTGCCCTC | This |
| (KRE5) | | study |
| i-Fw | | |
| RNAi | AAAGGGCCCTCATGTCGGTGCGGACAATC | This |
| (KRE5) | | study |
| i-Rv | | |
| RNAi | TTTCCCGGGTTACAACTCTTCATCGCCGT- | This |
| (KRE6) | CAC | study |
| SmaI-Fw | | |
| RNAi | AAACCCGGGAGATGACCACCGCAGACAATG | This |
| (KRE6) | | study |
| SmaI-Rv | | |
| GenR | AGCACGTACTCGGATGGAAG | This |
| probe-Fw | | study |
| GenR | CCTCAGAAGAACTCGTCAAGAAG | This |
| probe-Rv | | study |
| HyhR | TGAACTCACCGCGACGTCTG | Oliveira- |
| probe-Fw | | Garcia |
| | | & |
| | | Deising, |
| | | 2013 |
| HyhR | GAGCTGATGCTTTGGGCCGA | Oliveira- |
| probe-Rv | | Garcia |
| | | & |
| | | Deising, |
| | | 2013 |
| NatR | CTCTTGACGACACGGCTTAC | Oliveira- |
| probe-Fw | | Garcia |
| | | & |
| | | Deising, |
| | | 2013 |
| NatR | GGCAGGGCATGCTCATGTAG | Oliveira- |
| probe-Rv | | Garcia |
| | | & |
| | | Deising, |
| | | 2013 |
| CgGLS1 | GCTACTATGACGAATCGGGCTAT | Oliveira- |
| qRT-Fw | | Garcia |
| | | & |
| | | Deising, |
| | | 2013 |
| CgGLS1 | GCCATTGTTGCCGTTGTTG | Oliveira- |
| qRT-Rv | | Garcia |
| | | & |
| | | Deising, |
| | | 2013 |
| CgACTqR | TCCTACGAGCTTCCTGACGG | Krijger |
| T-F1 | | et al., |
| | | 2008. |
| CgACTqR | CCGCTCTCAAGACCAAGGAC | Krijger |
| T-R1 | | et al., |
| | | 2008. |
| CgH3- | CGAGATCCGTCGCTACCAGA | Krijger |
| qRT.F1 | | et al., |
| | | 2008. |
| CgH3- | GGAGGTCGGACTTGAAGTCCT | Krijger |
| qRT.R1 | | et al., |
| | | 2008. |
| CgITS2- | TGAACGCGAGCTAACTTGACA | Behr |
| q1 | | et al., |
| | | 2009. |
| CgITS2- | GGGCATCGAAGATGGAGGA | Behr |
| q2 | | et al., |
| | | 2009. |
| CgTKRE5 | TCTGCGGCCGCTCAGAAAGAGCCGGTAG- | This |
| NotI-Rv | AAG | study |
| CgTKRE6 | TCTCCCGGGTCAGAAAGAGCCGGTAGAAG | This |
| SmaI-Fw | | study |
| CgTKRE6 | TTTCCCGGGCAGGGCCTTACCTCGATACC | This |
| SmaI-Rv | | study |
| ZmTerp | CGTGGACACATGACGGAAAGAG | Oliveira- |
| S10_RT- | | Garcia |
| Fw | | & |
| | | Deising, |
| | | 2013. |
| ZmTerp | CTCGCTGATCTCATTCTCGTAGTG | Oliveira- |
| S10_RT- | | Garcia |
| Rv | | & |
| | | Deising, |
| | | 2013. |
| ZmTerp | TGTACAATGCGGCTCACATGGC | Oliveira- |
| S07_RT- | | Garcia |
| Fw | | & |
| | | Deising, |
| | | 2013. |
| ZmTerp | CTGACCTCGCCTTTGCTGATG | Oliveira- |
| S07_RT- | | Garcia |
| Rv | | & |
| | | Deising, |
| | | 2013. |
| ZmTerp | GATGCTGCGTCAGCGTCAGAAG | Oliveira- |
| S03_RT- | | Garcia |
| Fw | | & |
| | | Deising, |
| | | 2013. |
| ZmTerp | TCGAAGAAGTGGTCGATGCAGAG | Oliveira- |
| S03_RT- | | Garcia |
| Rv | | & |
| | | Deising, |
| | | 2013. |
| ZmTerp | GTCCGTACTTTGGCGGCATCATC | Oliveira- |
| RS02_T- | | Garcia |
| Fw | | & |
| | | Deising, |
| | | 2013. |
| ZmTerp | TCCGTTTGACGTCCGTCGTCAG | Oliveira- |
| S02_RT- | | Garcia |
| Rv | | & |
| | | Deising, |
| | | 2013. |
| ZmGluc | AGGCGCCATTGCTCAGACAGATAG | Oliveira- |
| 079_RT- | | Garcia |
| Fw | | & |
| | | Deising, |
| | | 2013. |
| ZmGluc | AGCCTCGAGCCATTCCGTGAAG | Oliveira- |
| 079_RT- | | Garcia |
| Rv | | & |
| | | Deising, |
| | | 2013. |
| ZmGluc | GTGCTCGCATAGTCGCATCGTTAG | Oliveira- |
| 262_RT- | | Garcia |
| Fw | | & |
| | | Deising, |
| | | 2013. |
| ZmGluc | CAGCAGCGCCTTCATGGTTGTC | Oliveira- |
| 262_RT- | | Garcia |
| Rv | | & |
| | | Deising, |
| | | 2013. |
| ZmGluc | CTCTACTAGAGGGCGGGTATAGAG | Oliveira- |
| P23_RT- | | Garcia |
| Fw | | & |
| | | Deising, |
| | | 2013. |
| ZmGluc | GCCGACAGAAAGACAGGAGCAAAC | Oliveira- |
| P23_RT- | | Garcia |
| Rv | | & |
| | | Deising, |
| | | 2013. |
| ZmGluc | CAGTGGACGCAGCAGCACATC | Oliveira- |
| P15_RT- | | Garcia |
| Fw | | & |
| | | Deising, |
| | | 2013. |
| ZmGluc | GGTCCGCGTGGTTCTTGAACTC | Oliveira- |
| P15_RT- | | Garcia |
| Rv | | & |
| | | Deising, |
| | | 2013. |
| ZmGluc | GCGGCGTACAACAACAACGTG | Oliveira- |
| 561_RT- | | Garcia |
| Fw | | & |
| | | Deising, |
| | | 2013. |
| ZmGluc | TGAACATGGCGAACAGGTAGGTC | Oliveira- |
| 561_RT- | | Garcia |
| Rv | | & |
| | | Deising, |
| | | 2013. |
| ZmGluc | CTGCAGAAGACGCTGGACTAC | Oliveira- |
| 714_RT- | | Garcia |
| Fw | | & |
| | | Deising, |
| | | 2013. |
| ZmGluc | GTAGGAGCAGTGGGCCTTGAC | Oliveira- |
| 714_RT- | | Garcia |
| Rv | | & |
| | | Deising, |
| | | 2013. |
| ZmGluc | CTGTTCGAGTTCGCCGACAAG | Oliveira- |
| 678_RT- | | Garcia |
| Fw | | & |
| | | Deising, |
| | | 2013. |
| ZmGluc | TCGACGACGTAGTCCAGGTACTC | Oliveira- |
| 678_RT- | | Garcia |
| Rv | | & |
| | | Deising, |
| | | 2013. |
| ZmGluc | CTGCCCACCAACTCCTTCTTC | Oliveira- |
| 782_RT- | | Garcia |
| Fw | | & |
| | | Deising, |
| | | 2013. |
| ZmGluc | GGTGGATGTACTCGGGCATGTC | Oliveira- |
| 782_RT- | | Garcia |
| Rv | | & |
| | | Deising, |
| | | 2013. |
| ZmGluc | CTTCCCGGCGAAGCCGATG | Oliveira- |
| 837_RT- | | Garcia |
| Fw | | & |
| | | Deising, |
| | | 2013. |
| ZmGluc | CAACTACAAGTACGCGCCGTTC | Oliveira- |
| 837_RT- | | Garcia |
| Rv | | & |
| | | Deising, |
| | | 2013. |
| ZmChit | GTCACCGGCTCCTTCTTCAAC | Oliveira- |
| 633_ | | Garcia |
| RT-Fw | | & |
| | | Deising, |
| | | 2013. |
| ZmChit | GCTCCGGGTGTAGAAGTTCTTG | Oliveira- |
| 633_ | | Garcia |
| RT-Rv | | & |
| | | Deising, |
| | | 2013. |
| ZmPero | CGTCCCATCCTACGATCCAAAG | Oliveira- |
| x182_ | | Garcia |
| RT-Fw | | & |
| | | Deising, |
| | | 2013. |
| ZmPero | TCTCGACAGCCTCGCTGTACTC | Oliveira- |
| x182_ | | Garcia |
| RT-Rv | | & |
| | | Deising, |
| | | 2013. |
| ZmPero | TCCGCCTCCACTTCCATGACTG | Oliveira- |
| x648_ | | Garcia |
| RT-Fw | | & |
| | | Deising, |
| | | 2013. |
| ZmPero | ATCGCGTCGATCACCTCGTACC | Oliveira- |
| x648_ | | Garcia |
| RT-Rv | | & |
| | | Deising, |
| | | 2013. |
| ZmPero | CGCAGCTCTCGGCAGATTTCTAC | Oliveira- |
| x816_ | | Garcia |
| RT-Fw | | & |
| | | Deising, |
| | | 2013. |
| ZmPero | TCGACGACGCCCAAGATGATCTTC | Oliveira- |
| x816_ | | Garcia |
| RT-Rv | | & |
| | | Deising, |
| | | 2013. |
| ZmPero | AGTTGGCTACCAGCCCTCTGTATG | Oliveira- |
| x871_ | | Garcia |
| RT-Fw | | & |
| | | Deising, |
| | | 2013. |
| ZmPero | GACCTGTGCTGCGTTGTTATAC | Oliveira- |
| x871_ | | Garcia |
| RT-Rv | | & |
| | | Deising, |
| | | 2013. |
| ZmPero | TGGTCAAGTACCACGTCGCCAAG | Oliveira- |
| x954_ | | Garcia |
| RT-Fw | | & |
| | | Deising, |
| | | 2013. |
| ZmPero | GACGAAGCAGTCGTGGAAGATGAG | Oliveira- |
| x954_ | | Garcia |
| RT-Rv | | & |
| | | Deising, |
| | | 2013. |
| ZmPero | GAGATGACGACCGCTCCCATTG | Oliveira- |
| x365_ | | Garcia |
| RT-Fw | | & |
| | | Deising, |
| | | 2013. |
| ZmPero | AGCGGGCTTATGTTGCCCATC | Oliveira- |
| x365_ | | Garcia |
| RT-Rv | | & |
| | | Deising, |
| | | 2013. |
| ZmPero | TTCCTGATGCCACCAAGGGTTC | Oliveira- |
| x346_ | | Garcia |
| RT-Fw | | & |
| | | Deising, |
| | | 2013. |
| ZmPero | GAGGGCAACGATGTCCTGATCAC | Oliveira- |
| x346_ | | Garcia |
| RT-Rv | | & |
| | | Deising, |
| | | 2013. |
| ZmPero | AGGGAGTACGTCCACCGTATTG | Oliveira- |
| x109_ | | Garcia |
| RT-Fw | | & |
| | | Deising, |
| | | 2013. |
| ZmPero | TGGCCATCTTGGTCATGGACTC | Oliveira- |
| x109_ | | Garcia |
| RT-Rv | | & |
| | | Deising, |
| | | 2013. |
| ZmPero | ACACAAGAAGCTGTCAGAGCTAGG | Oliveira- |
| x456_ | | Garcia |
| RT-Fw | | & |
| | | Deising, |
| | | 2013. |
| ZmPero | CATGCGACAACTGCGGTAGCAAC | Oliveira- |
| x456_ | | Garcia |
| RT-Rv | | & |
| | | Deising, |
| | | 2013. |
| ZmPero | CGGTGTTCGAGGTGATGGGCTAC | Oliveira- |
| x731_ | | Garcia |
| RT-Fw | | & |
| | | Deising, |
| | | 2013. |
| ZmPero | GCAGCAGTATGAGCGCCATGTTG | Oliveira- |
| x731_ | | Garcia |
| RT-Rv | | & |
| | | Deising, |
| | | 2013. |
| ZmSTKi | GCACGTTCTCCTCGTCAAACTG | Oliveira- |
| n276_ | | Garcia |
| RT-Fw | | & |
| | | Deising, |
| | | 2013. |
| ZmSTKi | CAGCCAGCCGAAGCTGATTATG | Oliveira- |
| n276_ | | Garcia |
| RT-Rv | | & |
| | | Deising, |
| | | 2013. |
| ZmUGT | AGAAAGCAAGGCGTGGCTGGAC | Oliveira- |
| 079_ | | Garcia |
| RT-Fw | | & |
| | | Deising, |
| | | 2013. |
| ZmUGT | TGCCGTAGAGGCCATCGGCTATC | Oliveira- |
| 079_ | | Garcia |
| RT-Rv | | & |
| | | Deising, |
| | | 2013. |
| ZmShi | TCAGGAATCCGGTGATCCTTATGC | Oliveira- |
| k590_ | | Garcia |
| RT-Fw | | & |
| | | Deising, |
| | | 2013. |
| ZmShi | CTCTGGCATCAGCATTAGCATACG | Oliveira- |
| k590_ | | Garcia |
| RT-Rv | | & |
| | | Deising, |
| | | 2013. |
| ZmGer | AGGCCATCGCCATGACGCTCTTC | Oliveira- |
| mLP1_ | | Garcia |
| RT-Fw | | & |
| | | Deising, |
| | | 2013. |
| ZmGer | TGGGTGCGAAGTTTGCCTTGATG | Oliveira- |
| mLP1_ | | Garcia |
| RT-Rv | | & |
| | | Deising, |
| | | 2013. |
| ZmSTK | GTCCCTTCGTTGCTGTGTCTTCTG | Oliveira- |
| in99_ | | Garcia |
| RT-Fw | | & |
| | | Deising, |
| | | 2013. |
| ZmSTK | GAATTGTCGCATCGCACCAGTTTG | Oliveira- |
| in99_ | | Garcia |
| RT-Rv | | & |
| | | Deising, |
| | | 2013. |
| ZmSTK | TATCGTCCACGTCACGACCGAATG | Oliveira- |
| in74_ | | Garcia |
| RT-Fw | | & |
| | | Deising, |
| | | 2013. |
| ZmSTK | CGGACCTGCCTACCTTTGCTAAC | Oliveira- |
| in74_ | | Garcia |
| RT-Rv | | & |
| | | Deising, |
| | | 2013. |
| ZmSTK | GGCGGTGTTCCAGTTCCAGTTCC | Oliveira- |
| in50_ | | Garcia |
| RT-Fw | | & |
| | | Deising, |
| | | 2013. |
| ZmSTK | CTAGCTGACAGGAGAGCGCATGAC | Oliveira- |
| in50_ | | Garcia |
| RT-Rv | | & |
| | | Deising, |
| | | 2013. |
| ZmSTK | CCTGTTTATCGGTCGGCTTCATGG | Oliveira- |
| in77_ | | Garcia |
| RT-Fw | | & |
| | | Deising, |
| | | 2013. |
| ZmSTK | AATGTTCGACTCGGCGATCCTATG | Oliveira- |
| in77_ | | Garcia |
| RT-Rv | | & |
| | | Deising, |
| | | 2013. |
| ZmTFZ | CAAGAGGCTGTGCCAGGAGATCG | Oliveira- |
| nfing | | Garcia |
| 653_ | | & |
| RT-Fw | | Deising, |
| | | 2013. |
| ZmTFZ | CGTCGGTGAACTTGGGATCCTTGG | Oliveira- |
| nfing | | Garcia |
| 653_ | | & |
| RT-Rv | | Deising, |
| | | 2013. |
| ZmWRKY | GGGAGATTATTGTGACGCCTGAG | Oliveira- |
| 62/ | | Garcia |
| 149_ | | & |
| RT-Fw | | Deising, |
| | | 2013. |
| ZmWRKY | CAGCTTCTCGACGAACTCGGAATC | Oliveira- |
| 62/ | | Garcia |
| 149_ | | & |
| RT-Rv | | Deising, |
| | | 2013. |
| ZmCellu | TGGGCCTTCACCTTCGTCATCAC | Oliveira- |
| Synt145_ | | Garcia |
| RT-Fw | | & |
| | | Deising, |
| | | 2013. |
| ZmCellu | GCCAGAGGTAGTAGCCGTCGAAC | Oliveira- |
| Synt145_ | | Garcia |
| RT-Rv | | & |
| | | Deising, |
| | | 2013. |
| ZmCellu | CATCCTGCTGGCCTCGATCTTCTC | Oliveira- |
| Synt8/ | | Garcia |
| 631_ | | & |
| RT-Fw | | Deising, |
| | | 2013. |
| ZmCellu | CAGTTGCAGTCCAGGCCACACTC | Oliveira- |
| Synt8/ | | Garcia |
| 631_ | | & |
| RT-Rv | | Deising, |
| | | 2013. |
| ZmCellu | TCTCGAAGCTGACAGGGCCAACG | Oliveira- |
| Synt567_ | | Garcia |
| RT-Fw | | & |
| | | Deising, |
| | | 2013. |
| ZmCellu | AAGGAACAGCCCAATGAACACCTC | Oliveira- |
| Synt567_ | | Garcia |
| RT-Rv | | & |
| | | Deising, |
| | | 2013. |
| ZmCalmo | CGCGGTGCTGTCGTCGCTGGG | Oliveira- |
| dul673_ | | Garcia |
| RT-Fw | | & |
| | | Deising, |
| | | 2013. |
| ZmCalmo | ATCTGGCGGAACTCGCGGAAGTC | Oliveira- |
| dul673_ | | Garcia |
| RT-Rv | | & |
| | | Deising, |
| | | 2013. |

*sequence overlaps and restriction sites are underlined

Cloning of the Plant Transformation Vector p7U-ubi RGA2intronII_HIGS_GLRG_05611 into Corn

In order to transform a HIGS construct directed against the Colletotrichum graminicola gene Kre5 into corn the plant transformation vector p7U-ubi_RGA2intronII_HIGS_GLRG_05611 (FIG. 22) was created. The HIGS cassette of the vector was composed of a hairpin construct which contained a 500 bp fragment of the Kre5 gene in sense orientation and the same DNA fragment in antisense orientation. The Kre5 fragments were separated by the RGA2intronII. After transcription of the hairpin construct by the corn ubiquitin promoter and splicing of the intron a double stranded Kre5 RNA molecule will be formed in the plant which is suitable for the generation of siRNAs.

In a first step the coding region of the Kre5 gene (GLRG_05611) was amplified from position 3680-4135 by PCR. PCR was done using the primers S2264 (CTGGATCCTGGTGAC-CTTCAAGTGGCCTCA) (SEQ ID NO: 366) and S2265 (GCCCCGGGGACTGTGAATGGG-GATCT) (SEQ ID NO: 367) which contained an additional BamHI (S2264) and XmaI (S2265) site for subcloning of the PCR fragment. PCR was done using genomic DNA of C. graminicola as template and the Phusion PCR polymerase for amplification (annealing temperature 55° C.).

The S2264-S2265 PCR product was purified by agarose gel electrophoresis, digested with the restriction enzymes BamHI and XmaI and subcloned into the vector pGGubi_RGA2intronII (FIG. 23), which was also treated with BamHI and XmaI. The resulting vector pGGubi_RGA2intronII_HIGS_GLRG_05611_sense contained the Kre5 gene in sense orientation upstream of the RGA2 intron. Cloning was done in the E. coli strain NEB5α.

In a second step the coding region of the Kre5 gene (GLRG_05611) was amplified from position 3680-4135 by PCR using the primers S2266 (CTCGATCGTGGTGAC-CTTCAAGTGGCCTCA) (SEQ ID NO: 368) and S2267 (GCAAGCTTGACTGTGAATGGGGATCT) (SEQ ID NO: 369) which contained an additional PvuI (S2266) and HindIII site (S2267). The S2266-S2267 PCR product was also purified by agarose gel electrophoresis, digested with the restriction enzymes PvuI and HindIII and subcloned as Kre5 antisense fragment into the vector pGGubi_RGA2intronII-HIGS-GLRG_05611_sense (FIG. 24), which was also pretreated with PvuI and HindIII. The resulting vector pGGubi_RGA2intronII_HIGS_GLRG_05611 (FIG. 24) contained the Kre5 gene in sense orientation upstream of the RGA2 intron and the Kre5 gene in antisense orientation downstream of the RGA2 intron. Cloning was done in the E. coli strain NEB5α.

In a third step the Kre5 hairpin cassette under the expression control of the corn ubiquitin promoter and the nopaline synthase gene terminator was subcloned as a Sfil fragment into the Sfil site of the binary vector p7U. The resulting vector p7U-ubi_RGA2intronII_HIGS_GLRG_05611 (FIG. 22) was transformed into the Agrobacterium strain GV2260 and used for the generation of transgenic corn plants.

Transformation of Corn

Corn transformation was done as described by Ishida et al. 2007 (Nature Protocols 2(7):1614-21). In brief immature embryos of the corn inbred line A188 were transformed by Agrobacterium tumefaciens mediated transformation using LS medium and the selection agent phosphinothricin.

Cloning of the Transcriptional Silencing Test Vector pABM Ubiluci GLRG 05611

In order to analyze the transcriptional silencing activity of the transformed corn lines against the C. graminicola Kre5 (GLRG_05611) gene the HIGS test vector pABM_ubiluci_GLRG_05611 (FIG. 25) was constructed. In this vector the Kre5 HIGS target sequence is located in the transcribed non-translated 3′-region of the luciferase gene. The transcription of this construct will result in a hybrid transcript encoding a luciferase and a non-translated partial Kre5 fragment. The expression of the reporter gene is sensitive to the presence of siRNAs which are directed against the Kre5 DNA fragment of the reporter gene construct. RNA degradation initiated by the Kre5 siRNAs will finally continue into the luciferase coding region by induction of secondary siRNAs targeting the sequences upstream of the Kre5 target sequence.

The coding region of the Kre5 gene from position 3680-4135 was amplified with the primers S2473 (CTAAGCTTTGGTGACCTTCAAGTGGCCTCA) (SEQ ID NO: 370) and S2247 (CCGTCGACGACTGTGAATGGGGATCT) (SEQ ID NO: 371), which included a HindIII (S2473) and a SalI (S2247) recognition site. The S2473-S2247 PCR product was inserted as a HindIII-SalI fragment into the corresponding recognition sites of the vector pABM_ubiluci to create pABM_ubiluci_GLRG_05611 (FIG. 25).

Detection of transcriptional silencing activity in HIGS corn lines

In order to detect transcriptional silencing activity in HIGS corn lines and to preselect promising HIGS lines the vector pABM_ubiluci_GLRG_05611 was transiently expressed with the normalization vector p70S-Ruc, a fusion between the doubled 35S-promoter and the coding sequence of the Renilla reniformis luciferase (Schmidt et al., 2004, Plant Mol. Biol., 55: 835-852). The transient biolistic experiments were done as described (Schmidt et. al 2004) by using the PDS-1000/He system (Biorad). The vector pABM_ubiluci_GLRG_05611 were mixed with the internal standard p70S-Ruc in a ratio of 1:1 (w:w). DNA was precipitated onto gold microcarriers (Au Typ 200-03; Heraeus, Hanau, Germany).

Leaf stripes of T0 corn lines were put on MS+0.4 M mannitol agar plates and bombarded with the DNA coated microcarriers, using a pressure of 1550 psi and a distance between the stopping screen and the leaf sample of 12 cm in a vacuum of 28.5 inches Hg. After bombardment, leaf samples were incubated for 16 h at 25° C. in the light. Activities of both luciferases were quantified using the dual luciferase assay (Promega). Relative reportergene activity was calculated as following:

(Photinus value_{(reportergene construct)}−Photinus value_{(without DNA)}/Renilla value_{(normalization construct)}−Renilla value_{(without DNA)})×100. The given mean was the average of 6 replicates.

Cloning and Transfer of the Plant Transformation Vector p6U-355-MgKRE5-355 into Wheat

In order to transform a HIGS construct against the Mycoshaerella graminicola (Zymoseptoria tritici) gene KRE5 into wheat the plant transformation vector p6U-35S-MgKRE5-35S (FIG. 26) was created. In a first step a 401 bp large fragment of the coding region of the KRES gene was amplified by PCR from position 3521-3921. PCR was done with the Phusion PCR polymerase for amplification according standard protocols using the primers S2237 (CTACTAGTCATGGCAACAGCGTACGGTTTC) and S2238 (ATGTCGACGTAATTCTGCCGCAGCCGATCAC) which contained an additional SpeI (S2237) and SalI (S2238) site for subcloning of the PCR fragment. Cloning was done in the E. coli strain NEB5a and the binary vector was transformed into the Agrobacterium strain GV2260 for wheat transformation. The KRE5 fragment of p6U-35S-MgKRE5-35S is inserted between two inverse oriented 35S promoters. The orientation of the 35S promoters allows the generation of double-stranded RNA from the KRE5 gene which is a substrate for the Dicer enzyme of wheat.

Wheat transformation was done as described by Ishida et al., 2015 (Methods Mol Biol. 2015; 1223:189-98) using immature embryos of healthy plants of the spring wheat cultivar Taifun grown in a well-conditioned greenhouse. T1 seeds of the primary wheat transformants were generated under greenhouse conditions.

Enhanced Septoria Blotch Resistance of a Wheat HIGS_{KRE5} Line

using the PDS-1000/He system (Biorad). The vector pABM_ubiluci_GLRG_05611 were mixed with the internal standard p70S-Ruc in a ratio of 1:1 (w:w). DNA was precipitated onto gold microcarriers (Au Typ 200-03; Heraeus, Hanau, Germany).

Leaf stripes of T0 corn lines were put on MS+0.4 M mannitol agar plates and bombarded with the DNA coated microcarriers, using a pressure of 1550 psi and a distance between the stopping screen and the leaf sample of 12 cm in a vacuum of 28.5 inches Hg. After bombardment, leaf samples were incubated for 16 h at 25° C. in the light. Activities of both luciferases were quantified using the dual luciferase assay (Promega). Relative reportergene activity was calculated as following:

(Photinus value_{(reportergene construct)}−Photinus value_{(without DNA)}/Renilla value_{(normalization construct)}−Renilla value_{(without DNA)})×100. The given mean was the average of 6 replicates.

Cloning and Transfer of the Plant Transformation Vector p6U-355-MgKRE5-355 into Wheat

In order to transform a HIGS construct against the Mycoshaerella graminicola (Zymoseptoria tritici) gene KRE5 into wheat the plant transformation vector p6U-35S-MgKRE5-35S (FIG. 26 top) was created. In a first step a 401 bp large fragment of the coding region of the KRES gene was amplified by PCR from position 3521-3921. PCR was done with the Phusion PCR polymerase for amplification according standard protocols using the primers S2237 (CTACTAGTCATGGCAACAGCGTACGGTTTC) and S2238 (ATGTCGACGTAATTCTGCCGCAGCCGATCAC) which contained an additional SpeI (S2237) and SalI (S2238) site for subcloning of the PCR fragment. Cloning was done in the E. coli strain NEB5α and the binary vector was transformed into the Agrobacterium strain GV2260 for wheat transformation. The KRE5 fragment of p6U-35S-MgKRE5-35S is inserted between two inverse oriented 35S promoters. The orientation of the 35S promoters allows the generation of double-stranded RNA from the KRE5 gene which is a substrate for the Dicer enzyme of wheat.

Wheat transformation was done as described by Ishida et al., 2015 (Methods Mol Biol. 2015; 1223:189-98) using immature embryos of healthy plants of the spring wheat cultivar Taifun grown in a well-conditioned greenhouse. T1 seeds of the primary wheat transformants were generated under greenhouse conditions.

Enhanced Septoria blotch Resistance of a Wheat HIGS_{KRE5} Line

T1 seeds of wheat HIGS_{KRE5} lines were planted in the greenhouse and transgenic homozygous, transgenic heterozygous and non-transgenic lines were identified by qPCR (TaqMan assay). In the case of a single T-DNA integration a 1:2:1 segregation pattern according the mendelian rules was observed.

Beside the cultivar Taifun, which was used for transformation, the non-transgenic progenitors of the HIGS_{KRE5} lines were collected and included as a non-transgenic segregant control into the resistance assay. In addition the German spring wheat lines Aurum and Passat were included as references. The heterozygous and homozygous T1 plants of each HIGS_{KRE5} lines were combined for the Septoria blotch assay. The resistance assay was done with twelve transgenic plants of each line in a randomized design in the greenhouse. After the appearance of the flag leaf the plants were spray inoculated at two times with 50.000 spores/ml of Mycosphaerealla graminicola (Zymoseptoria tritici). A disease scoring was made for the F (flag) and the F-1 leaf. The appearance of symptoms (0-100%) was scored 21, 26, 31 and 35 days after inoculation. An area under disease progression curve (AUDPC) was calculated until 35 days after inoculation.

The HIGS_{KRE5} line WA-601-T-035 revealed an enhanced Septoria blotch resistance in the assay (FIG. 26 below). The transformation genotype Taifun and the non-transgenic segregants showed an AUDPC value of 316 and 330, respectively. The AUDPC value of the HIGS_{KRE5} line WA-601-T-035 was only 237. This value was also below the AUDPC value of the the two reference lines, Aurum and Passat, which were included into the assay.

1-15. (canceled)

16. A transgenic plant or a part thereof comprises a transgene, wherein the transgene comprises a DNA capable of expressing an inhibitory nucleic acid molecule capable of inhibiting the expression of one or both of a KRE5 gene and a KRE6 gene in a fungus.

17. The transgenic plant or the part thereof of claim 16, wherein the transgene comprises an expression cassette comprising the DNA.

18. The transgenic plant or the part thereof of claim 16, wherein the transgene is stably integrated into the genome of the transgenic plant or the part thereof, or wherein the transgene is present on a vector in the transgenic plant or the part thereof.

19. The transgenic plant or the part thereof of claim 16, wherein the inhibitory nucleic acid molecule is an antisense RNA or dsRNA.

20. The transgenic plant or the part thereof of claim 16,
wherein the DNA encodes an RNA molecule in sense direction and an RNA molecule in antisense direction,
wherein the RNA molecule in sense direction or the RNA molecule in antisense direction are substantially complementary to the KRE5 gene, the KRE6 gene, a part of the KRE5 gene, or a part of the KRE6 gene,
wherein the RNA molecule in the antisense direction is substantially reverse complementary to the RNA molecule in the sense direction, and
wherein the RNA molecule in the sense direction and the RNA molecule in the antisense direction are able to form a dsRNA.

21. The transgenic plant or the part thereof of claim 16, wherein the length of the antisense RNA, the dsRNA, the RNA molecule in sense direction or the RNA molecule in antisense direction is at least 15 contiguous nucleotides.

22. The transgenic plant or the part thereof of claim 21, wherein the length of the antisense RNA, the dsRNA, the RNA molecule in sense direction or the RNA molecule in antisense direction is 400-500 contiguous nucleotides.

23. The transgenic plant or the part thereof of claim 16, wherein the expression of the inhibitory nucleic acid molecule is controlled by a promoter.

24. An inhibitory nucleic acid molecule capable of inhibiting the expression of one or both of a KRE5 gene and a KRE6 gene in a fungus.

25. A DNA comprising the inhibitory nucleic acid molecule of claim 24.

26. An expression cassette comprising the DNA of claim 25.

27. A vector comprising (a) the DNA of claim 25, or (b) an expression cassette comprising the DNA.

28. A method of producing the transgenic plant or the part thereof of claim 16 comprising:
introducing into at least a cell of the plant (a) a DNA of claim 25, or (b) a dsRNA capable of inhibiting the expression of one or both of the KRE5 gene and the KRE6 gene in the fungus; and
regenerating the transgenic plant from the at least one cell.

29. A method of conferring fungal resistance to a plant or the part thereof comprising:
introducing into the plant or the part thereof (a) a DNA of claim 25, (b) an expression cassette comprising the DNA, or (c) a dsRNA capable of inhibiting the expression of one or both of the KRE5 gene and the KRE6 gene in the fungus; and
causing expression of the DNA or the expression cassette.

30. A method of inhibiting the expression of one or both of a KRE5 gene and a KRE6 gene in a fungus, comprising applying the DNA of claim 25 or an expression cassette comprising the DNA to the fungus or to a plant or a part thereof.

31. A method of inhibiting the expression of one or both of a KRE5 gene and a KRE6 gene in a fungus, comprising contacting a plant or a part thereof with the DNA of claim 25 or an expression cassette comprising the DNA.

32. A method of inactivating a fungus comprising applying the DNA of claim 25 or an expression cassette comprising the DNA to the fungus or to a plant or a part thereof.

33. A method of protecting a plant against an infection by a fungus comprising applying the DNA of claim 25 or an expression cassette comprising the DNA to the fungus or to a plant or a part thereof.

34. A composition comprising a fungus, wherein the fungus comprises (a) the DNA of claim 25, (b) an expression cassette comprising the DNA, or a vector comprising the DNA or the expression cassette.
